(19) United States
(12) Patent Application Publication
(10) Pub. No.: US 2005/0014765 A1
(43) Pub. Date: Jan. 20, 2005

(54) Aryl-heteroaromatic products, compositions comprising them and use

(75) Inventors: Patrick Mailliet, Fontenay Sous Bois (FR); Alain Le Brun, Vigneux (FR); Fabienne Thompson, Paris (FR); Gilles Tiraboschi, Chevilly Larue (FR)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma S.A., Antony (FR)

(21) Appl. No.: 10/859,740

(22) Filed: Jun. 3, 2004

(30) Foreign Application Priority Data
Jun. 4, 2003 (FR) 0306719
May 6, 2004 (FR) 0404889

(51) Int. Cl. A61K 31/496; A61K 31/4439; C07D 413/02

(52) U.S. Cl. 514/254.02; 514/340; 544/369; 546/269.1; 546/271.4

(57) ABSTRACT

Aryl-heteroaromatic products, compositions comprising them and use. The present invention relates to novel chemical compounds, particularly to novel aryl-heteroaromatic products, to compositions comprising them, and to their use as medicaments, in particular in oncology.

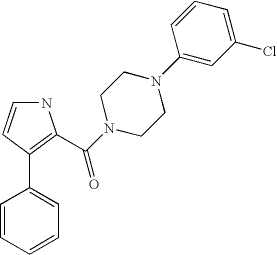

### CROSS-REFERENCE TO RELATED APPLICATIONS

This application claims the benefit of priority from French Patent Application No. 0306719, filed Jun. 4, 2003, as well as the benefit of French Patent Application No. 0404889, filed May 6, 2004.

The present invention relates to novel chemical compounds, particularly novel aryl-heteroaromatic products, to compositions comprising them and to their use as medicaments.

More particularly, according to a first aspect, the invention relates to novel aryl-heteroaromatic products exhibiting an anticancer activity and in particular an inhibitory activity with regard to tubulin polymerization.

The aryl-heteroaromatic products concerned with here correspond to the following general formula (I):

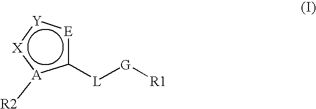

Products in which A=C, X=N, Y=C-phenyl and E=CH are known:

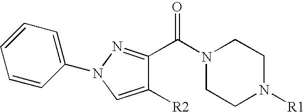

| CAS Number | R1 | R2 |
|---|---|---|
| 380442-50-2 | 2-chlorophenyl | 2-thienyl |
| 375394-90-4 | 2-methoxyphenyl | 2-thienyl |
| 380221-88-5 | 2-methoxyphenyl | 3,4-dimethoxyphenyl |
| 372106-93-9 | 2-methoxyphenyl | 3-pyridyl |
| 379266-21-4 | 3-trifluoromethylphenyl | 4-methoxyphenyl |
| 368861-17-0 | 2-methoxyphenyl | 4-chlorophenyl |
| 375395-06-5 | phenyl | 2-thienyl |
| 367512-29-6 | phenyl | phenyl |
| 367512-13-8 | 2-methoxyphenyl | 4-fluorophenyl |
| 366492-22-0 | phenyl | 4-chlorophenyl |

An isoxazole derivative is also known: 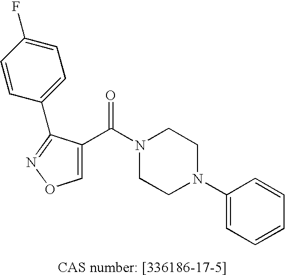
Di Stilo et al., in Medicinal Chemistry Research (1994), 3(9), 554-566, disclose prazosin analogs of use for their vasodilating properties:

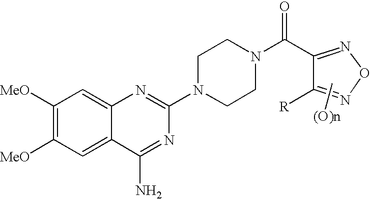

The 1,2,5-oxadiazole is optionally in the form of an N-oxide. R is phenyl.

Products identified [CAS numbers]: [157066-46-1], [157066-44-9], [157066-43-8] and [157066-42-7]. Patent application WO 01/19798 claims heterocyclic compounds of use as factor Xa inhibitors for the treatment, for example, of thrombosis and for inhibiting the clotting of biological samples. The products disclosed are not included in the definition of the products according to the invention, with the exception of the following compound:

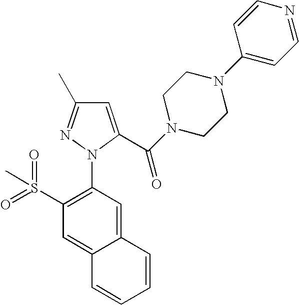

Ermondi et al., in Farmaco, 53, 519 (1998), disclose prazosin analogs which are potential α₁-adrenoreceptor inhibitors. Just one prazosin analog is a 5-(4-(heteroaryl)piperazinocarbonyl)-1-phenylpyrazole:

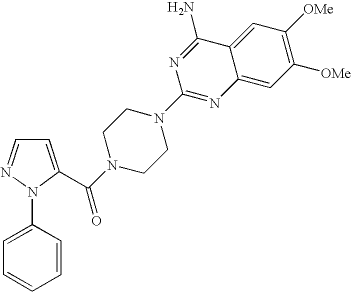

In point of fact, surprisingly, it has been found that products corresponding to the following general formula (I) exhibit a significant inhibitory activity with regard to tubulin polymerization: 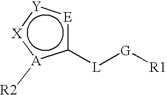
in which:
1) A is N or C;
2) L-G-R1 is chosen from 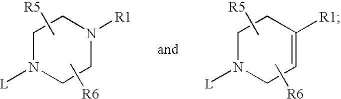
3) X and Y are chosen independently from CR3, N, NR3, O or S;
4) E is CR4, N, NR4 or S;
5) R1 and R2 are selected independently from the group consisting of aryl, heteroaryl, substituted aryl and substituted heteroaryl;
6) L is selected from the group consisting of C═O, C═S and C═N(R7);
7) R3 and R4 are selected independently from the group consisting of H, alkyl, cycloalkyl, cycloalkylene, heterocyclyl, O—R7, S—R7, SO—R7, SO₂—(R7), N(R7)(R8), halogen, aryl, heteroaryl, substituted cycloalkyl, substituted aryl, substituted heteroaryl and substituted alkyl;
8) R5 and R6 are selected independently from the group consisting of H and (C₁-C₃)alkyl;
9) R7 and R8 are selected independently from the group consisting of H, (C₁-C₃)alkyl and substituted (C₁-C₃)alkyl;

in the racemic form, enriched in one enantiomer, enriched in one diastereoisomer, its tautomers, its prodrugs and its pharmaceutically acceptable salts, with the proviso that the product of formula (I) is not one of the following compounds:

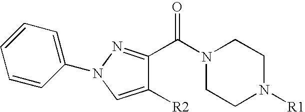

| CAS Number | R1 | R2 |
|---|---|---|
| 380442-50-2 | 2-chlorophenyl | 2-thienyl |
| 375394-90-4 | 2-methoxyphenyl | 2-thienyl |
| 380221-88-5 | 2-methoxyphenyl | 3,4-dimethoxyphenyl |
| 372106-93-9 | 2-methoxyphenyl | 3-pyridyl |
| 379266-21-4 | 3-trifluoromethylphenyl | 4-methoxyphenyl |
| 368861-17-0 | 2-methoxyphenyl | 4-chlorophenyl |
| 375395-06-5 | phenyl | 2-thienyl |
| 367512-29-6 | phenyl | phenyl |
| 367512-13-8 | 2-methoxyphenyl | 4-fluorophenyl |
| 366492-22-0 | phenyl | 4-chlorophenyl |

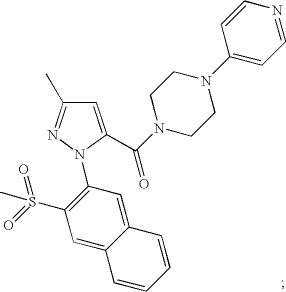

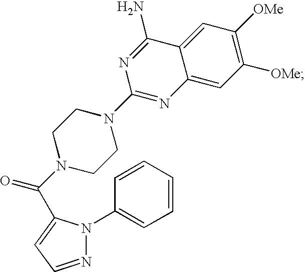

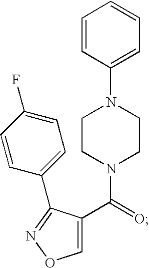

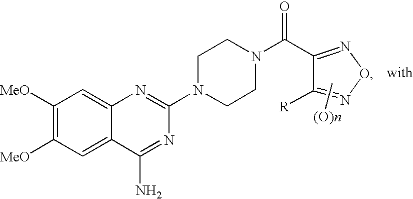

n = 0, 1 or 2 and R = phenyl.

Products of general formula (I) 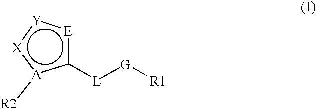
in which:
A is N or C;
and L-G-R1 is chosen from 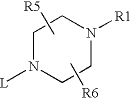
are preferred.

Products for which:
E is NR4 with R4 is H are preferred.

A preferred R1 substituent can be chosen from phenyl, phenyl substituted by at least one radical chosen from halogen, CF₃, CN, NO₂, (C₁-C₃)alkyl, O—R10, S—R10, N(R10)(R11), CO—O—R10, CO—N(R10)(R11), NH—CO—R10 in which R10 and R11 are chosen independently from H, (C₁-C₃)alkyl, halogenated (C₁-C₃)alkyl, (C₁-C₃)alkyl-OH, (C₁-C₃)alkyl-NH₂, (C₁-C₃)alkyl-COOH, (C₁-C₃)alkyl-OCH₃, (C₁-C₃)alkyl-NHCH₃, pyridyl, pyridyl substituted by at least one radical chosen from halogen, (C₁-C₃)alkyl, O—R12, S—R12 or N(R12)(R13) in which R12 and R13 are chosen independently from H or (C₁-C₃)alkyl.

More preferably, R1 will be phenyl substituted in the 3-position by halogen or (C₁-C₃)alkyl, (C₁-C₃)alkoxy, (C₁-C₃)alkylamino, CONH₂, CO—NH—(CH₂)₂—OH or NH—CO—CH₃; or 3-pyridyl; 2- or 3-pyridyl substituted by halogen, (C₁-C₃)alkyl or (C₁-C₃)alkoxy.

When R1 is substituted phenyl, preferred substitution combinations can be chosen from 2,3-disubstituted phenyl, 2,5-disubstituted phenyl, 3-substituted phenyl, 3,5-disubstituted phenyl and 3,4-disubstituted phenyl, more preferably from 3-substituted phenyl, 3,5-disubstituted phenyl and 3,4-disubstituted phenyl.

When R1 is 2-pyridyl, preferred substitutions are chosen from 4- or 6-substituted 2-pyridyl or 4,6-disubstituted 2-pyridyl.

When R1 is 3-pyridyl, preferred substitutions are 2- or 5-substituted 3-pyridyl.

Very preferably, R1 is phenyl substituted in the 3-position by a chloro radical or in the 3- and 5-positions by two methoxy radicals.

Very preferably, R1 is phenyl substituted in the 3-position by a cyano radical, a carboxamide radical, a methoxy radical or a hydroxymethyl radical.

A preferred R2 substituent can be chosen from phenyl, phenyl substituted by at least one radical chosen from halogen, alkyl, O—R10, S—R10 and N(R10)(R11), in which R10 and R11 are independently chosen from H, alkyl and halogenated alkyl; or 3-pyridyl.

According to a second aspect, the invention relates to pharmaceutical compositions comprising a product according to its first aspect, in combination with a pharmaceutically acceptable excipient.

A product according to the invention can advantageously be used as agent which inhibits tubulin polymerization, as agent which inhibits the proliferation of tumor cells, for promoting the breakup of clusters of cells originating from a vascular tissue, or for the manufacture of a medicament of use in treating a pathological condition, preferably cancer.

Generally, products of general formula (Ia) or (Ib) in accordance with the invention in which L is C(O) can be prepared by coupling a heteroarylcarboxylic acid substituted in the position ortho to the carboxyl functional group by an aryl or heteroaryl radical, of general formula (II), in which A, X, Y, E and R2 are defined as above, with respectively a piperazine derivative of general formula (IIIa) or a 1,2,3,6-tetrahydropyridine derivative of general formula (IIIb), in which R1 is defined as above, according to scheme 1:

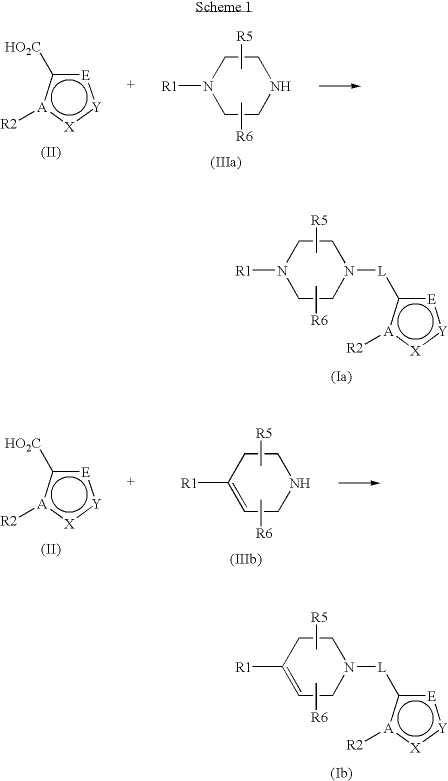

The heteroarylcarboxylic acids of general formula (II) in which A, X, Y, E and R2 are commercially available or can be obtained according to general synthetic methods known to a person skilled in the art.

The piperazine derivatives of general formula (IIIa) in which R1, R5 and R6 are defined as above are either commercially available or are prepared according to conventional methods known to a person skilled in the art.

Among these methods, N1-aryl(heteroaryl)ation, according to scheme 2, of piperazines carrying a protective group on the 4-nitrogen is particularly advantageous in the context of the invention:

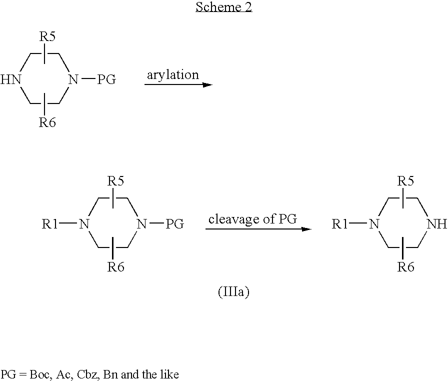

The aryl(heteroaryl)ation reaction of piperazines, generally of Hartwig-Buchwald type, can be carried out according to the conditions described in Biorg. Med. Chem. Lett., 11, 1375 (2001) or in Biorg. Med. Chem., 10, 3817 (2002).

Another method for the synthesis of aryl(heteroaryl)piperazines, particularly advantageous in the context of the invention, when R5 and R6 represent hydrogen atoms, consists of the reaction of an aryl(heteroaryl)amine with a bis(2-hydroxy- or 2-haloethyl)amine, at a temperature of greater than 100-120° C. according to scheme 3:

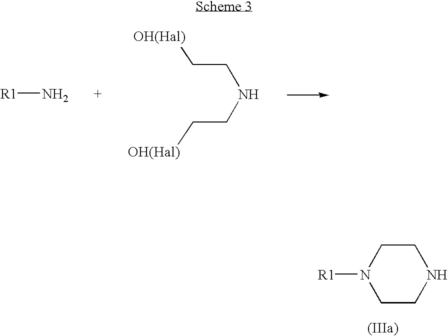

It is particularly advantageous to carry out the reaction in the presence of microwaves under the conditions described in Synth. Comm., 28, 1175 (1998) or in Tetrahedron Lett, 38, 6875 (1997).

The 1,2,3,6-tetrahydropyridine derivatives (IIIb) in which R1, R5 and R6 are defined as above are either commercially available or are prepared according to conventional methods known to a person skilled in the art.

Among these methods, the action, according to scheme 4, of an organometallic aryl(heteroaryl)derivative, such as an organomagnesium derivative, an organolithium derivative or an organocerium derivative, on a piperidin-4-one derivative, the nitrogen atom of which is substituted by a protective group, is particularly advantageous.

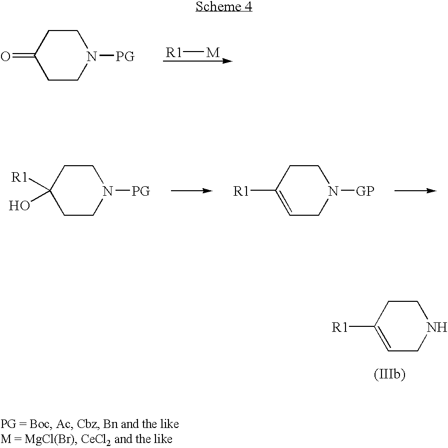

It is possible in particular to carry out the reaction under the conditions described in J. Med. Chem., 38, 1998 (1995) or in E.P. 306764 or in J. Med. Chem., 28, 311 (1985).

When R5 and R6 represent hydrogen atoms, Suzuki-type coupling of the pinacol ester of N-Boc-1,2,3,6-tetrahydropyridyl-4-boronic acid with an aryl or heteroaryl halide, preferably a bromide or an iodide, under the conditions described in Tetrahedron Lett, 41, 3705 (2000), according to scheme 5, is particularly advantageous in the context of the invention: it is understood that the Boc protective group can be replaced by any other protective group compatible with the reaction conditions and that the pinacol boronic ester can also be replaced by any other boron derivative, acid or ester, compatible with said conditions.

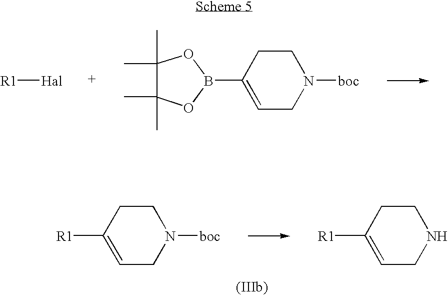

Generally, products of general formula (Ia) or (Ib) in accordance with the invention in which L is C(S) can be prepared by thionation of a compound of general formula (Ia) or (Ib) respectively, in which L is C(O), by any one of the thionation methods known to a person skilled in the art, the reaction being carried out according to scheme 6:

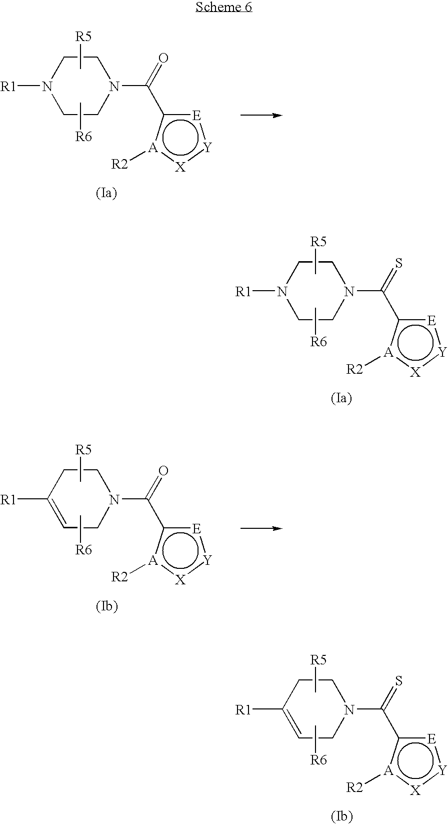

It is particularly advantageous in the context of the invention to carry out the thionation using Lawesson's reagent, the reaction being carried out according to Bull. Soc. Chim. Belg., 87, 293 (1978).

Generally, products of general formula (Ia) or (Ib) in accordance with the invention in which L is C(NH) can be prepared from the nitrites derived from the products of general formula (II), using the various methods known to a person skilled in the art, according to the reaction sequences of scheme 7:

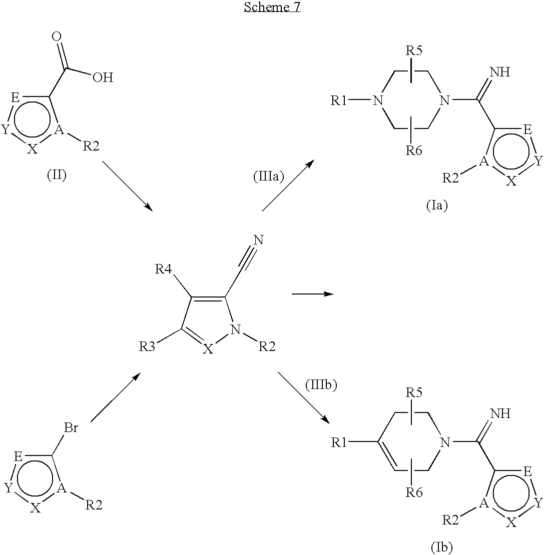

It is generally necessary to activate the not very reactive nitrile, either with aluminum chloride, the reaction being carried out according to J. Chem. Soc. 1947, 1110; or with cuprous iodide, the reaction being carried out according to Tetrahedron Lett., 34, 6395 (1993); or by converting nitrile to iminoether prior to the reaction with the piperazine or 1,2,3,6-tetrahydropyridine or piperidine derivative, the reaction being carried out according to Eur. J. Med. Chem., 24, 427 (1989).

Generally, products of general formula (Ia) in accordance with the invention in which L is C(NR7), with R7 the same as or different from the hydrogen atom, can be prepared from the products of general formula (Ia) in which L is C(O) and/or C(S), using the various methods known to a person skilled in the art, according to the reaction sequences of scheme 8:

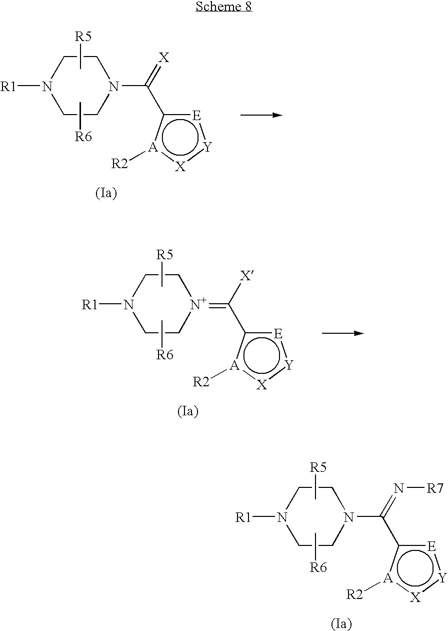

In the context of the invention, when X is an oxygen atom, it is particularly advantageous to successively react oxalyl chloride, which results in an intermediate in which X′ is a chlorine atom, and then an amine R7-NH₂, the reaction being carried out according to Pol. J. Chem., 58, 117 (1984), and, when X is a sulfur atom, to react first methyl iodide, which results in an intermediate in which X′ is a methylthio radical, and then an amine R7-NH₂, the reaction being carried out according to Eur. J. Med. Chem, 12, 365 (1977).

More specifically and more particularly advantageously in the context of the invention, products in accordance with the invention can also be prepared on a solid phase, according to reaction scheme 9:

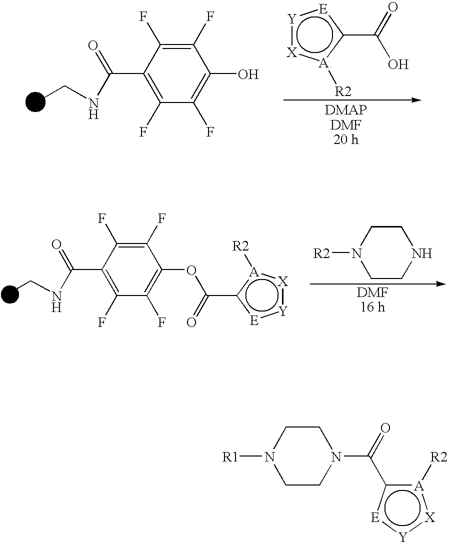

The general synthetic methods presented in schemes 1 to 9 illustrate, without implied limitation, possible preparations of the compounds of the invention. Numerous other synthetic routes can be used, in particular those described in:
Comprehensive Heterocyclic Chemistry, 5 (Part 4A), by A. Katritsky et al. (Pergamon Press).

The examples below illustrate, without implied limitation, the products of the invention. The various products are purified either as described in the examples or by LC/MS under the general conditions described below:

Purification by LC/MS:

The products were purified by LC/MS using a Waters FractionsLynx system composed of a Waters model 600 gradient pump, a Waters model 515 regeneration pump, a Waters Reagent Manager dilution pump, a Waters model 2700 auto-injector, two Rheodyne model LabPro valves, a Waters model 996 diode array detector, a Waters model ZMD mass spectrometer and a Gilson model 204 fraction collector. The system was controlled by the Waters FractionLynx software. Separation was carried out alternately on two Waters Symmetry columns (C₁₈, 5 μm, 19×50 mm, catalog reference 186000210), one column undergoing regeneration with a 95/5 (v/v) water/acetonitrile mixture comprising 0.07% (v/v) of trifluoroacetic acid, while the other column was being used for separation. The columns were eluted using a linear gradient of from 5% to 95% of acetonitrile comprising 0.07% (v/v) of trifluoroacetic acid in water comprising 0.07% (v/v) of trifluoroacetic acid, at a flow rate of 10 ml/min. At the outlet of the separation column, one-thousandth of the effluent is separated by means of an LC Packing Accurate, diluted with methyl alcohol at a flow rate of 0.5 ml/min and sent to the detectors, in a proportion of 75% to the diode array detector and the remaining 25% to the mass spectrometer. The rest of the effluent (999/1000) is sent to the fraction collector, where the flow is discarded for as long as the mass of the expected product is not detected by the FractionLynx software. The molecular formulae of the expected products are supplied to the FractionLynx software, which actuates the collection of the product when the mass signal detected corresponds to the ion [M+H]⁺ and/or to [M+Na]⁺. In certain cases, depending on the analytical LC/MS results, when an intense ion corresponding to [M+2H]⁺⁺ was detected, the value corresponding to half the calculated molecular mass (MW/2) is also supplied to the FractionLynx software. Under these conditions, the collection is also actuated when the mass signal for the ion [M+2H]⁺⁺ and/or [M+Na+H]⁺⁺ is detected. The products were collected in tared glass tubes. After collection, the solvents were evaporated in a Savant AES 2000 or Genevac HT8 centrifugal evaporator and the masses of the products were determined by weighing the tubes after evaporation of the solvents.

The LC/MS analyses were carried out on a Micromass model LCT device connected to an HP 1100 device. The abundance of the products was measured using an HP G1315A diode array detector over a wavelength range of 200-600 nm and a Sedex 65 light scattering detector. The mass spectra were acquired over a range of 180 to 800. The data were analyzed using the Micromass MassLynx software. Separation was carried out on a Hypersil BDS C18, 3 μm (50×4.6 mm) column, eluting with a linear gradient of from 5% to 90% of acetonitrile comprising 0.05% (v/v) of trifluoroacetic acid (TFA) in water comprising 0.05% (v/v) TFA, over 3.5 min at a flow rate of 1 ml/min. The total analysis time, including the period for re-equilibrating the column, is 7 min.

### EXAMPLE 1

[4-(3-Chlorophenyl)piperazin-1-yl](1-phenyl-1H-imidazol-5-yl)methanone

Stage 1: 3.5 g of ethyl 1-phenyl-1H-imidazol-5-ylcarboxylate, which can be prepared according to Tetrahedron Lett. (2000) 41, 5453-56, are dissolved in 50 ml of ethanol in a 100 ml three-necked flask, then 25 ml of water and 16.2 ml of an 85% aqueous potassium hydroxide solution are added and then the mixture is stirred for 20 hours at ambient temperature. After concentrating under reduced pressure, the reaction medium is taken up in 100 ml of water and then washed 3 times with 75 ml of diethyl ether. The aqueous phase is brought to pH=3-4 by addition of hydrochloric acid and washed 3 times with 100 ml of dichloromethane. The aqueous phase is concentrated under vacuum and the residue is taken up in 10 ml of methanol and then filtered. Finally, the filtrate is taken up in 25 ml of isopropyl ether and the product is filtered off and washed with 2 times 2 ml of isopropyl ether. 2.5 g of 1-phenyl-1H-imidazol-5-ylcarboxylic acid are thus obtained in the form of a brown solid, used as is in the following stage.

Stage 2: 342 μl of oxalyl chloride and a few drops of dimethylformamide are added successively to a solution of 0.5 g of 1-phenyl-1H-imidazol-5-yl-carboxylic acid in 25 ml of dichloromethane in a 100 ml three-necked flask under an argon atmosphere, and stirring is carried out for 2 hours at ambient temperature. The solution thus obtained is transferred into a dropping funnel and is added dropwise to a solution, cooled to 0° C. under an argon atmosphere, of 575 mg of 1-(3-chlorophenyl)piperazine in 25 ml of dichloromethane comprising 560 μl of triethylamine and 132 μl of 4-dimethylaminopyridine.

After stirring at ambient temperature for 20 hours, 20 ml of water are added and the organic phase is separated by settling, washed with water, dried over magnesium sulfate and concentrated under reduced pressure. The residue is purified by flash chromatography on silica gel (70-230 mesh), elution being carried out with dichloromethane, and then by crystallization from isopropyl ether. 280 mg of [4-(3-chlorophenyl)piperazin-1-yl](1-phenyl-1H-imidazol-5-yl)methanone are thus obtained in the form of light beige crystals, the characteristics of which are as follows:

Melting point (Kofler bench)=70° C.

¹H NMR spectrum (400 MHz, (CD₃)₂SO, at a temperature of 393 K, δ in ppm): 3.18 (t, J=5 Hz: 4H); 3.66 (t, J=5 Hz: 4H); 6.81 (ddd, J=8-2 and 1 Hz: 1H); 6.85 (ddd, J=8-2 and 1 Hz: 1H); 6.90 (t, J=2 Hz: 1H); 7.21 (t, J=8 Hz: 1H); 7.30 (tt, J=7.5 and 1.5 Hz: 1H); 7.40 (tt, J=7.5 and 1.5 Hz: 2H); 7.65 (dd, J=7.5 and 1.5 Hz: 2H); 7.72 (s: 1H).

### EXAMPLE 2

[4-(3-Chlorophenyl)piperazin-1-yl](5-phenyl-1,3-oxazol-4-yl)methanone

By carrying out the reaction as in stage 3 of example 1, but from, on the one hand, 500 mg of 5-phenyl-1,3-oxazol-4-ylcarboxylic acid and 0.25 ml of oxalyl chloride in 20 ml of dichloromethane and from, on the other hand, 0.48 ml of 1-(3-chlorophenyl)piperazine in 20 ml of dichloromethane comprising 0.75 ml of triethylamine, at ambient temperature for 20 hours. After purifying by flash chromatography on silica gel (70-230 mesh), elution being carried out with a mixture of ethyl acetate and cyclohexane (30-70 by volume), 0.83 g of [4-(3-chlorophenyl)piperazin-1-yl](5-phenyl-1,3-oxazol-4-yl)methanone is obtained in the form of a beige foam, the characteristics of which are as follows:

Mass spectrum (EI): m/z=367 (M⁺)

¹H NMR spectrum (300 MHz, (CD₃)₂SO, 6 in ppm): 3.12 (broad t, J=5 Hz: 2H); 3.30 (mt: 2H); 3.56 (broad t, J=5 Hz: 2H); 3.82 (broad t, J=5 Hz: 2H); 6.82 (dd large, J=8 and 2 Hz: 1H); 6.91 (dd, J=8 and 2 Hz: 1H); 6.97 (t, J=2 Hz: 1H); 7.24 (t, J=8 Hz: 1H); 7.46 (broad t, J=7.5 Hz: 1H); 7.52 (broad t, J=7.5 Hz: 2H); 7.76 (broad d, J=7.5 Hz: 2H); 8.60 (s: 1H).

### EXAMPLE 3

[4-(3-Chlorophenyl)piperazin-1-yl](4-phenyl-1H-imidazol-5-yl)methanone

Stage 1: By carrying out the reaction as in stage 2 of example 1 but from 350 mg of ethyl 4-phenyl-1H-imidazol-5-ylcarboxylate, which can be prepared according to Tetrahedron Lett. (1994), 35, 1635-38, and 1.6 ml of an 85% aqueous potassium hydroxide solution in 5 ml of ethanol and 2.5 ml of water, 218 mg of 4-phenyl-1H-imidazol-5-ylcarboxylic acid are obtained in the form of a beige solid, used as is in the following stage.

Stage 2: By carrying out the reaction as in stage 2 of example 1 but from, on the one hand, 188 mg of 4-phenyl-1H-imidazol-5-ylcarboxylic acid and 128 μl of oxalyl chloride in 10 ml of dichloromethane and from, on the other hand, 216 mg of 1-(3-chlorophenyl)piperazine in 10 ml of dichloromethane comprising 210 μl of triethylamine and 5 μl of 4-dimethylaminopyridine, at ambient temperature for 20 hours. After purifying by flash chromatography on silica gel (70-230 mesh), elution being carried out with a mixture of dichloromethane and methanol (95-5 by volume), 150 mg of [4-(3-chlorophenyl)piperazin-1-yl](4-phenyl-1H-imidazol-5-yl)methanone are obtained in the form of a beige foam, the characteristics of which are as follows:

Melting point (Kofler bench)=208° C.

¹H NMR spectrum (300 MHz, (CD₃)₂SO, 6 in ppm): 3.10 (unresolved peak: 4H); 3.64 (broad t, J=5 Hz: 4H); 6.83 (dd, J=8 and 2 Hz: 1H); 6.90 (dd, J=8 and 2 Hz: 1H); 6.96 (t, J=2 Hz: 1H); 7.24 (t, J=8 Hz: 1H); from 7.35 to 7.50 (mt: 4H); 7.54 (broad t, J=7.5 Hz: 2H); 8.09 (broad s: 1H).

### EXAMPLE 4

[4-(3-Chlorophenyl)piperazin-1-yl](3-phenylthiophen-2-yl)methanone

By carrying out the reaction of stage 2 of example 1, but from, on the one hand, 77 mg of 3-phenylthiophen-2-ylcarboxylic acid, which can be prepared according to J. Org. Chem. (1967), 32, 463-4, and 35 μl of oxalyl chloride in 4 ml of dichloromethane and from, on the other hand, 62 μl of 1-(3-chlorophenyl)piperazine in 4 ml of dichloromethane comprising 62 μl of triethylamine, at ambient temperature for 36 hours. After purifying by crystallization from the minimum amount of dichloromethane, 50 mg of [4-(3-chlorophenyl)piperazin-1-yl](3-phenylthiophen-2-yl)methanone are obtained in the form of an off-white solid, the characteristics of which are as follows:

Mass spectrum (EI): m/z=382 (M⁺)

¹H NMR spectrum (400 MHz, (CD₃)₂SO, at a temperature of 363 K, δ in ppm): 2.92 (unresolved peak: 4H); 3.43 (mt: 4H); from 6.75 to 6.85 (mt: 3H); 7.20 (t, J=8.5 Hz: 1H); from 7.30 to 7.40 (mt: 1H); 7.32 (d, J=5 Hz: 1H); from 7.40 to 7.55 (mt: 4H); 7.75 (d, J=5 Hz: 1H).

### EXAMPLE 5

[4-(3-Methoxyphenyl)piperazin-1-yl][2-(4-chlorophenyl)furan-3-yl]methanone

24.6 mg of 1-(3-dimethylaminopropyl)-3-ethylcarbodiimide hydrochloride (EDCl) and 1.6 mg of 1-hydroxybenzotriazole hydrate (HOBT) are added to a solution of 26 mg of 3-(4-chlorophenyl)furan-2-ylcarboxylic acid, which can be obtained according to Coll. Czech. Chem. Commun. (1989) 54, 215-24, in 5 ml of dichloromethane. After stirring at ambient temperature for 10 minutes, 24.7 mg of 1-(3-methoxyphenyl)piperazine are added and then this reaction mixture is stirred at ambient temperature for 24 hours. After purifying by chromatography on 6 g of fine silica, elution being carried out with a mixture of cyclohexane and ethyl acetate (50-50 by volume), 8.9 mg of [4-(3-methoxyphenyl)piperazin-1-yl][2-(4-chlorophenyl)furan-3-yl]methanone are obtained in the form of a colorless lacquer, the characteristics of which are as follows:

Mass spectrum (EI): m/z=396 (M⁺)

¹H NMR spectrum (300 MHz, (CD₃)₂SO, 6 in ppm): 2.99 (unresolved peak: 2H); 3.24 (unresolved peak: 2H); 3.42 (mt: 2H); 3.71 (s: 3H); 3.81 (unresolved peak: 2H); 6.43 (broad d, J=8 Hz: 1H); 6.46 (mt: 1H); 6.52 (broad d, J=8 Hz: 1H); 6.76 (d, J=1.5 Hz: 1H); 7.14 (t, J=8 Hz: 1H); 7.55 (d, J=8 Hz: 2H); 7.67 (d, J=8 Hz: 2H); 7.90 (d, J=1.5 Hz: 1H).

### EXAMPLE 6

[4-(3-Chlorophenyl)piperazin-1-yl](3-phenyl-1H-pyrrol-2-yl)methanone

Stage 1: 99 mg of ethyl 3-phenyl-1H-pyrrol-2-ylcarboxylate, which can be prepared according to Austr. J. Chem. (1994), 47, 969-74, are dissolved in 5 ml of tetrahydrofuran. 96.5 mg of lithium hydroxide monohydrate are then added and stirring is carried out at ambient temperature for 20 hours. After concentrating under reduced pressure, the residue is dissolved in 5 ml of water and a 1N hydrochloric acid solution is added until a pH of 6 is reached. The precipitate formed is filtered off and dried under vacuum. 80 mg of 3-phenyl-1H-pyrrol-2-ylcarboxylic acid are obtained in the form of a white solid used as is in the following stage.

Stage 2: The reaction is carried out as in stage 2 of example 1 but in a 10 ml Stern tube under argon and from 80 mg of 3-phenyl-1H-pyrrol-2-ylcarboxylic acid and 112 μl of oxalyl chloride in 5 ml of dichloromethane. Unlike stage 2 of example 1, the reaction medium is concentrated under reduced pressure, then the acid chloride thus obtained is dissolved in 5 ml of tetrahydrofuran, then 76.3 mg of 1-(3-chlorophenyl)piperazine and 81.8 μl of triethylamine are added, and then the mixture is stirred at ambient temperature for 20 hours. The crude product is purified by LC/MS according to the procedure described above. 120 mg of [4-(3-chlorophenyl)piperazin-1-yl](3-phenyl-1H-pyrrol-2-yl)-methanone are thus obtained in the form of a beige foam, the characteristics of which are as follows:

Mass spectrum (EI): m/z=365 (M⁺)

¹H NMR spectrum (300 MHz, (CD₃)₂SO, 6 in ppm): 2.90 (unresolved peak: 4H); 3.42 (unresolved peak: 4H); 6.34 (t, J=2.5 Hz: 1H); 6.80 (mt: 2H); 6.86 (mt: 1H); 6.95 (t, J=2.5 Hz: 1H); from 7.10 to 7.25 (mt: 2H); from 7.30 to 7.40 (mt: 4H); 11.50 (unresolved peak: 1H)

### EXAMPLE 7

[4-(3-Chlorophenyl)piperazin-1-yl](1-methyl-3-phenyl-1H-pyrrol-2-yl)-methanone

80 mg of [4-(3-chlorophenyl)piperazin-1-yl](3-phenyl-1H-pyrrol-2-yl)-methanone, obtained in example 6, are dissolved in 5 ml of dimethylformamide in a 10 ml Stern tube, then 10.5 mg of sodium hydride are added and, after 1 hour, 13.64 μl of methyl iodide are added. After stirring at ambient temperature for 10 hours, the reaction medium is concentrated under reduced pressure and the residue is then taken up with 5 ml of dichloromethane. The insoluble impurities are filtered off and the filtrate is concentrated under reduced pressure. The residue is purified by LC/MS under the conditions described above. 80 mg of [4-(3-chlorophenyl)piperazin-1-yl](1-methyl-3-phenyl-1H-pyrrol-2-yl)methanone are obtained in the form of a beige foam, the characteristics of which are as follows:

Mass spectrum (EI): m/z=379 (M⁺)

¹H NMR spectrum (300 MHz, (CD₃)₂SO, δ in ppm): from 2.80 to 3.40 (large unresolved peak: 6H); from 3.50 to 3.80 (large unresolved peak: 2H); 3.61 (s: 3H); 6.32 (d, J=3 Hz: 1H); 6.79 (broad d, J=8 Hz: 2H); 6.85 (mt: 1H); 6.95 (d, J=3 Hz: 1H); from 7.10 to 7.25 (mt: 2H); from 7.25 to 7.40 (mt: 4H).

### EXAMPLE 8

[4-(3,5-Dimethoxyphenyl)piperazin-1-yl](3-phenyl-1H-pyrrol-2-yl)methanone

The reaction is carried out as in stage 2 of example 1 but from 214 mg of 3-phenyl-1H-pyrrol-2-ylcarboxylic acid and 210 μl of oxalyl chloride in 5 ml of dichloromethane. The acid chloride thus obtained is dissolved in 5 ml of tetrahydrofuran, 161 mg of 1-(3,5-dimethoxyphenyl)piperazine and 153 μl of triethylamine are added and then the mixture is stirred at ambient temperature for 20 hours. After purifying by LC/MS according to the procedure described above, 51 mg of [4-(3,5-dimethoxyphenyl)piperazin-1-yl](3-phenyl-1H-pyrrol-2-yl)methanone are obtained in the form of a beige foam, the characteristics of which are as follows:

Mass spectrum (EI): m/z=391 (M⁺)

¹H NMR spectrum (300 MHz, (CD₃)₂SO, 6 in ppm): from 2.70 to 2.95 (unresolved peak: 4H); from 3.20 to 3.50 (unresolved peak: 4H); 3.68 (s: 6H); 5.98 (s: 3H); 6.34 (mt: 1H); 6.93 (mt: 1H); 7.22 (mt: 1H); from 7.55 to 7.40 (mt: 4H); 11.49 (unresolved peak: 1H).

### EXAMPLE 9

[4-(3,5-Dimethoxyphenyl)piperazin-1-yl](4-phenyl-1H-imidazol-5-yl)-methanone

The reaction is carried out as in example 5 but from, on the one hand, 1 g of 4-phenyl-1H-imidazol-5-ylcarboxylic acid, obtained in stage 1 of example 3, and from 1.2 g of 1-(3,5-dimethoxyphenyl)piperazine in 90 ml of dichloromethane, in the presence of 1.1 g of 1-(3-dimethylaminopropyl)-3-ethylcarbodiimide hydrochloride (EDCl) and 0.79 g of 1-hydroxybenzotriazole hydrate (HOBT), with stirring at ambient temperature for 48 hours. After purifying by flash chromatography on silica gel (60; 30-75 μm), elution being carried out with a mixture of dichloromethane and ethanol (95-5 by volume), and then crystallization from 25 ml of diisopropyl ether, 1.3 g of [4-(3,5-dimethoxyphenyl)piperazin-1-yl](4-phenyl-1H-imidazol-5-yl)methanone are obtained in the form of white crystals, the characteristics of which are as follows:

Mass spectrum (EI): m/z=392 (M⁺)

Melting point (Kofler bench)=196° C.

### EXAMPLE 10

[4-(Pyridin-3-yl)piperazin-1-yl](4-phenyl-1H-imidazol-5-yl)methanone

The reaction is carried out as in example 5 but, on the one hand, from 100 mg of 4-phenyl-1H-imidazol-5-ylcarboxylic acid, obtained in stage 1 of example 3, and from 87 mg of 1-(pyridin-3-yl)piperazine, which can be prepared according to Tetrahedron Lett. (1998), 39(7), 617-20, in 10 ml of dichloromethane, in the presence of 112 mg of 1-(3-dimethylaminopropyl)-3-ethylcarbodiimide hydrochloride (EDCl) and 79 mg of 1-hydroxybenzotriazole hydrate (HOBT), with stirring at ambient temperature for 20 hours. After purifying by flash chromatography on silica gel (60; 30-75 μm), elution being carried out with a mixture of dichloromethane and methanol (95-5 by volume), and then crystallization from 5 ml of diisopropyl ether, 100 mg of [4-(pyridin-3-yl)piperazin-1-yl](4-phenyl-1H-imidazol-5-yl)methanone are obtained in the form of a white foam, the characteristic of which is follows:

Mass spectrum (EI): m/z=333 (M⁺)

### EXAMPLE 11

[4-(3,5-Dimethoxyphenyl)piperazin-1-yl](1-phenyl-1H-imidazol-5-yl)-methanone hydrochloride

The reaction is carried out as in example 5 but, on the one hand, from 580 mg of 1-phenyl-1H-imidazol-5-ylcarboxylic acid, obtained in stage 1 of example 1, and from 685 mg of 1-(3,5-dimethoxyphenyl)piperazine in 50 ml of dichloromethane, in the presence of 650 mg of 1-(3-dimethylaminopropyl)-3-ethylcarbodiimide hydrochloride (EDCl) and 460 mg of 1-hydroxybenzotriazole hydrate (HOBT), with stirring at ambient temperature for 48 hours. After purifying by flash chromatography on silica gel (60; 30-75 μm), elution being carried out with a mixture of dichloromethane and ethanol (95-5 by volume), 950 mg of [4-(3,5-dimethoxyphenyl)piperazin-1-yl]-(1-phenyl-1H-imidazol-5-yl)methanone are obtained, which is then converted to hydrochloride by recrystallization from a mixture of 50 ml of ethyl acetate and 2.5 ml of a 1M hydrochloric acid solution in diethyl ether. 900 mg of [4-(3,5-dimethoxyphenyl)piperazin-1-yl](1-phenyl-1H-imidazol-5-yl)methanone hydrochloride are thus obtained in the form of white crystals, the characteristic of which is as follows:

Melting point (Kofler bench)=268° C.

### EXAMPLE 12

[4-(3-Acetylaminophenyl)piperazin-1-yl](4-phenyl-1H-imidazol-5-yl)methanone

The reaction is carried out as in stage 2 of example 1 but from 189 mg of 4-phenyl-1H-imidazol-5-ylcarboxylic acid, obtained in stage 1 of example 3, and 94 μl of oxalyl chloride in 20 ml of dichloromethane, comprising a few drops of DMF, and then from 76.3 mg of 1-(3-acetylaminophenyl)piperazine, which can be prepared according to Tetrahedron Lett. (1994), 35(40), 7331-34, 281 μl of triethylamine and 12 mg of 4-dimethylaminopyridine, with stirring at ambient temperature for 20 hours. After purifying by flash chromatography on silica gel (60; 30-75 μm), elution being carried out with a mixture of dichloromethane and methanol (97.5-2.5 by volume), and then crystallization from 5 ml of diisopropyl ether, 180 mg of [4-(3-acetylaminophenyl)piperazin-1-yl](4-phenyl-1H-imidazol-5-yl)methanone are obtained in the form of a beige solid, the characteristic of which is as follows:

Mass spectrum (EI): m/z=389 (M⁺)

### EXAMPLE 13

[4-(3-Cyanophenyl)piperazin-1-yl](1-phenyl-1H-imidazol-5-yl)methanone

The reaction is carried out as in example 5 but, on the one hand, from 376 mg of 1-phenyl-1H-imidazol-5-ylcarboxylic acid, obtained in stage 1 of example 1, and from 520 mg of 1-(3-cyanophenyl)piperazine, which can be prepared according to Tetrahedron Lett. (2000), 56(24), 4107-10, in 34 ml of dichloromethane, in the presence of 422 mg of 1-(3-dimethylaminopropyl)-3-ethylcarbodiimide hydrochloride (EDCl) and 297 mg of 1-hydroxybenzotriazole hydrate (HOBT) with stirring at ambient temperature for 20 hours. After purifying by flash chromatography on silica gel (60; 30-75 μm), elution being carried out with a mixture of dichloromethane and methanol (97.5-2.5 by volume), and then crystallization from 5 ml of diisopropyl ether, 650 mg of [4-(3-cyanophenyl)piperazin-1-yl](1-phenyl-1H-imidazol-5-yl)methanone are obtained in the form of a white solid, the characteristic of which is as follows:

Mass spectrum (EI): m/z=357 (M⁺)

### EXAMPLE 14

[4-(3-Cyanophenyl)piperazin-1-yl](4-phenyl-1H-imidazol-5-yl)methanone

The reaction is carried as in example 5 but, on the one hand, from 200 mg of 4-phenyl-1H-imidazol-5-ylcarboxylic acid, obtained in stage 1 of example 3, and from 276 mg of 1-(3-cyanophenyl)piperazine, which can be prepared according to Tetrahedron Lett. (2000), 56(24), 4107-10, in 34 ml of dichloromethane, in the presence of 224 mg of 1-(3-dimethylaminopropyl)-3-ethylcarbodiimide hydrochloride (EDCl) and 158 mg of 1-hydroxybenzotriazole hydrate (HOBT), with stirring at ambient temperature for 20 hours. After purifying by flash chromatography on silica gel (60; 30-75 μm), elution being carried out with a mixture of dichloromethane and methanol (97.5-2.5 by volume), and then crystallization from 5 ml of diisopropyl ether, 350 mg of [4-(3-cyanophenyl)piperazin-1-yl](4-phenyl-1H-imidazol-5-yl)methanone are obtained in the form of a white solid, the characteristic of which is as follows:

Mass spectrum (EI): m/z=357 (M⁺)

### EXAMPLE 15

[4-(3-Chlorophenyl)piperazin-1-yl](4-phenyl-1H-pyrrol-3-yl)methanone

The reaction is carried out as in example 5 but, on the one hand, from 562 mg of 4-phenyl-1H-pyrrol-3-ylcarboxylic acid, which can be prepared according to Med. Chem. Res. (1997), 7(2), 98-108, and from 590 mg of 1-(3-chlorophenyl)piperazine in 90 ml of dichloromethane, in the presence of 632 mg of 1-(3-dimethylaminopropyl)-3-ethylcarbodiimide hydrochloride (EDCl) and 446 mg of 1-hydroxybenzotriazole hydrate (HOBT), with stirring at ambient temperature for 72 hours. After purifying by flash chromatography on silica gel (60; 30-75 μm), elution being carried out with a mixture of cyclohexane and ethyl acetate (50-50 by volume), and then crystallization from 15 ml of diisopropyl ether, 900 mg of [4-(3-chlorophenyl)piperazin-1-yl](4-phenyl-1H-pyrrol-3-yl)methanone are obtained in the form of a light beige solid, the characteristic of which is as follows:

Mass spectrum (EI): m/z=365 (M⁺)

### EXAMPLE 16

[4-(3,5-Dimethoxyphenyl)piperazin-1-yl](4-phenyl-1H-pyrrol-3-yl)methanone

The reaction is carried out as in example 5 but, on the one hand, from 562 mg of 4-phenyl-1H-pyrrol-3-ylcarboxylic acid, which can be prepared according to Med. Chem. Res. (1997), 7(2), 98-108, and from 667 mg of 1-(3,5-dimethoxyphenyl)piperazine in 90 ml of dichloromethane, in the presence of 632 mg of 1-(3-dimethylaminopropyl)-3-ethylcarbodiimide hydrochloride (EDCl) and 446 mg of 1-hydroxybenzotriazole hydrate (HOBT), with stirring at ambient temperature for 72 hours. After purifying by flash chromatography on silica gel (60; 30-75 μm), elution being carried out with a mixture of cyclohexane and ethyl acetate (60-40 by volume), and then crystallization from 15 ml of diisopropyl oxide, 1 g of [4-(3,5-dimethoxyphenyl)piperazin-1-yl]-(4-phenyl-1H-pyrrol-3-yl)methanone are obtained in the form of a light beige solid, the characteristic of which is as follows:

Mass spectrum (EI): m/z=391 (M⁺)

### EXAMPLE 17

[4-(3-Carboxamidophenyl)piperazin-1-yl](1-phenyl-1H-imidazol-5-yl)methanone

A solution of 600 mg of [4-(3-cyanophenyl)piperazin-1-yl](1-phenyl-1H-imidazol-5-yl)methanone, obtained in example 13, in 20 ml of methanol comprising 0.5N aqueous sodium hydroxide is refluxed for 72 hours in a 100 ml three-necked flask, under an argon atmosphere. At the beginning of heating, the methanol solution comprises 3.7 ml of a 0.5N aqueous sodium hydroxide solution and then, after refluxing for 20 hours, an additional 3.7 ml of a 0.5N aqueous sodium hydroxide solution are added. After cooling, the solvent is concentrated and the residue is then dissolved in 100 ml of dichloromethane and 20 ml of methanol. Washing is then carried out with a saturated ammonium chloride solution, and the organic phase is separated by settling, dried over magnesium sulfate and concentrated under reduced pressure. The crude product is purified by recrystallization from 5 ml of ethyl acetate. 180 mg of [4-(3-carboxamidophenyl)piperazin-1-yl](1-phenyl-1H-imidazol-5-yl)methanone are thus obtained in the form of white crystals, the characteristic of which is as follows:

Mass spectrum (EI): m/z=375 (M⁺)

### EXAMPLE 18

[4-(3,5-Dimethoxyphenyl)piperazin-1-yl](1-methyl-4-phenyl-1H-pyrrol-3-yl)methanone hydrochloride

The reaction is carried out as in example 5 but, on the one hand, from 201 mg of 1-methyl-4-phenyl-1H-pyrrol-3-ylcarboyxlic acid which can be prepared according to Med. Chem. Res. (1997), 7(2), 98-108, and from 222 mg of 1-(3,5-dimethoxyphenyl)piperazine in 20 ml of dichloromethane, in the presence of 211 mg of 1-(3-dimethylaminopropyl)-3-ethylcarbodiimide hydrochloride (EDCl) and 148 mg of 1-hydroxybenzotriazole hydrate (HOBT), with stirring at ambient temperature for 72 hours. After purifying the base by flash chromatography on silica gel (60; 30-75 μm), elution being carried out with a mixture of cyclohexane and ethyl acetate (50-50 by volume), and then crystallization in hydrochloride form from 5 ml of dichloromethane and 1 ml of a 1M hydrochloric acid solution in diethyl ether, 400 mg of [4-(3,5-dimethoxyphenyl)piperazin-1-yl](1-methyl-4-phenyl-1H-pyrrol-3-yl)methanone hydrochloride are obtained in the form of white crystals, the characteristic of which is as follows:

Mass spectrum (EI): m/z=441 (M⁺)

### EXAMPLE 19

[4-(3-Chlorophenyl)piperazin-1-yl](2-mercapto-5-phenyl-1H-imidazol-4-yl)-methanone

The reaction is carried out as in example 5 but from 200 mg of 2-mercapto-5-phenyl-1H-imidazol-4-ylcarboxylic acid, which can be prepared according to Chem. Pharm. Bull. (1984), 32(7), 2536-43, and from 178.6 mg of 1-(3-chlorophenyl)piperazine in 15 ml of dichloromethane, in the presence of 192 mg of 1-(3-dimethylaminopropyl)-3-ethylcarbodiimide hydrochloride (EDCl) and 135 mg of 1-hydroxybenzotriazole hydrate (HOBT), with stirring at ambient temperature for 24 hours. After concentrating under reduced pressure, 20 ml of water are added. The precipitate formed is filtered off, washed successively with 3 times 20 ml of water and then twice 20 ml of diethyl ether, and dried. 260 mg of [4-(3-chlorophenyl)piperazin-1-yl](2-mercapto-5-phenyl-1H-imidazol-4-yl)methanone are thus obtained in the form of a ecru powder, the characteristics of which are as follows:

Melting point (Kofler bench)>260° C.

Mass spectrum (EI): m/z=398 (M⁺)

### EXAMPLE 20

[4-(3,5-Dimethoxyphenyl)piperazin-1-yl](2-mercapto-5-phenyl-1H-imidazol-4-yl)methanone

The reaction is carried out as in example 5 but from 200 mg of 2-mercapto-5-phenyl-1H-imidazol-4-yl-carboxylic acid, which can be prepared according to Chem. Pharm. Bull. (1984), 32(7), 2536-43, and from 202 mg of 1-(3,5-dimethoxyphenyl)piperazine in 15 ml of dichloromethane, in the presence of 192 mg of 1-(3-dimethylaminopropyl)-3-ethylcarbodiimide hydrochloride (EDCl) and 135 mg of 1-hydroxybenzotriazole hydrate (HOBT), with stirring at ambient temperature for 24 hours. After purifying by flash chromatography on silica gel (60; 30-75 μm), elution being carried out with a mixture of dichloromethane and methanol (95-5 by volume), 263 mg of 4-(3,5-dimethoxyphenyl)piperazin-1-yl](2-mercapto-5-phenyl-1H-imidazol-4-yl)methanone are obtained in the form of a yellow foam, the characteristics of which are as follows:

Mass spectrum (EI): m/z=424 (M⁺).

### EXAMPLE 21

[4-(3,5-Dimethoxyphenyl)piperazin-1-yl](2-phenyl-1H-pyrrol-3-yl)methanone

Stage 1: 1.2 g of ethyl 2-phenyl-1H-pyrrol-3-ylcarboxylate, which can be prepared according to J. Chem. Soc. Perkin Trans 1 1994 (17), 2355-56, are dissolved in 80 ml of ethanol and 19.5 ml of 1N aqueous sodium hydroxide solution in a 250 ml three-necked flask; the solution is brought to reflux for 48 hours. After concentrating the ethanol under reduced pressure, the reaction medium is dissolved in 25 ml of distilled water. The aqueous solution obtained is washed with 3 times 10 ml of ethyl acetate and then acidified by addition of 39.5 ml of a 1N aqueous hydrochloric acid solution. The precipitate formed is filtered off, washed 3 times with 5 ml of water, and then dried in an oven at 45° C. 1 g of 2-phenyl-1H-pyrrol-3-ylcarboxylic acid is thus obtained, which is used as is in the following step.

Stage 2: The reaction is carried out as in example 5 but from 375 mg of 2-phenyl-1H-pyrrol-3-ylcarboxylic acid, prepared above, and 440 mg of 1-(3,5-dimethoxyphenyl)piperazine in 30 ml of dichloromethane, in the presence of 420 mg of 1-(3-dimethylaminopropyl)-3-ethylcarbodiimide hydrochloride (EDCl) and 27 mg of 1-hydroxybenzotriazole hydrate (HOBT), with stirring at ambient temperature for 72 hours, and after 24 hours, 20 ml of dichloromethane will have been added. After purifying the base by flash chromatography on silica gel (60; 30-75 μm), elution being carried out with a mixture of dichloromethane and ethanol (95-5 by volume), 300 mg of [4-(3,5-dimethoxyphenyl)piperazin-1-yl](2-mercapto-5-phenyl-1H-imidazol-4-yl)-methanone are obtained in the form of a pale violet foam, the characteristics of which are as follows:

Mass spectrum (EI): m/z=391 (M⁺)

### EXAMPLE 22

[4-(3-Carboxamidophenyl)piperazin-1-yl](4-phenyl-1H-imidazol-5-yl)-methanone

The reaction is carried out as in example 5 but, on the one hand, from 189 mg of 4-phenyl-1H-imidazol-5-ylcarboxylic acid, obtained in stage 1 of example 3, and 278 mg of 1-(3-carboxamidophenyl)piperazine dihydrochloride, which can be prepared according to WO 98/00400, in 35 ml of dichloromethane, in the presence of 422 μl of triethylamine, 211 mg of 1-(3-dimethylaminopropyl)-3-ethylcarbodiimide hydrochloride (EDCl) and 148 mg of 1-hydroxybenzotriazole hydrate (HOBT), with stirring at ambient temperature for 20 hours. The precipitate obtained is filtered off, washed successively with 2 times 5 ml of dichloromethane, 2 times 20 ml of water, 2 times 20 ml of a saturated aqueous sodium hydrogen carbonate solution and then 2 times 20 ml of water. The precipitate is then formed into a paste in 10 ml of a mixture of ethyl acetate and diisopropyl ether (50-50 by volume). 230 mg of [4-(3-carboxamidophenyl)piperazin-1-yl](4-phenyl-1H-imidazol-5-yl)methanone are thus obtained in the form of a light beige solid, the characteristic of which is as follows:

Mass spectrum (EI): m/z=375 (M⁺)

### EXAMPLE 23

[4-(3,5-Dimethoxyphenyl)piperazin-1-yl](2-methylsulfanyl-5-phenyl-1H-imidazol-4-yl)methanone

210 mg of 4-(3,5-dimethoxyphenyl)piperazin-1-yl](2-mercapto-5-phenyl-1H-imidazol-4-yl)methanone, obtained in example 20, are dissolved in 10 ml of methanol in a 25 ml three-necked flask. 32 mg of sodium methoxide are then added, the mixture is stirred at ambient temperature for 10 minutes, a solution of 77.3 mg of methyl iodide is then added and the mixture is refluxed for 3 hours. A further 32 mg of sodium methoxide and 228 mg of methyl iodide are then added, and the mixture is then brought to reflux for 24 hours. The solvent is concentrated under reduced pressure and the reaction medium is taken up with 20 ml of water and extracted with 2 times 20 ml of ethyl acetate. The combined organic phases are dried over magnesium sulfate and concentrated under reduced pressure. The beige foam obtained (160 mg) is purified by flash chromatography on 25 g of silica gel (60; 30-75 μm), elution being carried out with a mixture of dichloromethane and methanol (95-5 by volume). By recovering the fraction eluted between 880 and 960 ml, 68 mg of [4-(3,5-dimethoxyphenyl)piperazin-1-yl](2-methylsulfanyl-5-phenyl-1H-imidazol-4-yl)methanone are obtained in the form of a white foam, the characteristics of which are as follows:

Mass spectrum (EI): m/z=438 (M⁺)

¹H NMR spectrum (300 MHz, (CD₃)₂SO, 6 in ppm):

### EXAMPLE 24

[4-(3,5-Dimethoxyphenyl)piperazin-1-yl](N-methyl-2-methylsulfanyl-5-phenyl-1H-imidazol-4-yl)methanone

By carrying out the reaction as in example 23, but recovering the fraction eluted between 420 and 500 ml, 27 mg of 4-(3,5-dimethoxyphenyl)piperazin-1-yl](N-methyl-2-methylsulfanyl-5-phenyl-1H-imidazol-4-yl)methanone are obtained in the form of a colorless white lacquer, the characteristics of which are as follows:

Mass spectrum (EI): m/z=452 (M⁺)

¹H NMR spectrum (300 MHz, (CD₃)₂SO, δ in ppm).

### EXAMPLE 25

[4-(3-Carboxamidophenyl)piperazin-1-yl](2-phenyl-1-H-pyrrol-3-yl)methanone

The reaction is carried out as in example 5 but from 195 mg of 2-phenyl-1H-pyrrol-3-ylcarboxylic acid, obtained in stage 1 of example 21, and from 280 mg of 1-(3-carboxamidophenyl)piperazine dihydrochloride, which can be prepared according to WO 98/00400, in 35 ml of dichloromethane, in the presence of 420 μl of triethylamine, 210 mg of 1-(3-dimethylaminopropyl)-3-ethylcarbodiimide hydrochloride (EDCl) and 150 mg of 1-hydroxybenzotriazole hydrate (HOBT), with stirring at ambient temperature for 20 hours. The precipitate obtained is filtered off, washed successively with 2 times 5 ml of dichloromethane, 2 times 20 ml of water, 2 times 20 ml of a saturated aqueous sodium hydrogen carbonate solution and then 2 times 20 ml of water. The precipitate is then purified by flash chromatography on silica gel (60; 30-75 μm), elution being carried out with a mixture of dichloromethane and ethanol (90-10 by volume). 125 mg of [4-(3-carboxamidophenyl)piperazin-1-yl](2-phenyl-1H-pyrrol-3-yl)methanone are thus obtained in the form of a beige solid, the characteristic of which is as follows:

Mass spectrum (EI): m/z=374 (M⁺)

### EXAMPLE 26

[4-(3-Chlorophenyl)piperazin-1-yl](2-phenyl-1H-pyrrol-3-yl)methanone

The reaction is carried out as in example 5 but from 189 mg of 2-phenyl-1H-pyrrol-3-ylcarboxylic acid, obtained in stage 1 of example 21, and from 200 mg of 1-(3-chlorophenyl)piperazine in 15 ml of dichloromethane, in the presence of 210 mg of 1-(3-dimethylaminopropyl)-3-ethylcarbodiimide hydrochloride (EDCl) and 13 mg of 1-hydroxybenzotriazole hydrate (HOBT), with stirring at ambient temperature for 72 hours. The precipitate obtained is filtered off, and washed successively with 2 times 5 ml of dichloromethane, 2 times 20 ml of water, 2 times 20 ml of a saturated aqueous sodium hydrogen carbonate solution and then 2 times 20 ml of water. The precipitate is then purified by flash chromatography on silica gel (60; 30-75 μm), elution being carried out with a mixture of dichloromethane and ethanol (95-5 by volume). 125 mg of [4-(3-chlorophenyl)piperazin-1-yl](2-phenyl-1H-pyrrol-3-yl)methanone are thus obtained in the form of a pinkish-beige solid, the characteristic of which is as follows:

Mass spectrum (EI): m/z=365 (M⁺)

### EXAMPLE 27

[4-(3,5-Dimethoxyphenyl)piperazin-1-yl](1-methyl-2-phenyl-1H-pyrrol-3-yl)-methanone

900 mg of [4-(3,5-dimethoxyphenyl)piperazin-1-yl](2-phenyl-1H-pyrrol-3-yl)methanone, prepared in example 21, are dissolved in 20 ml of pyridine in a 100 ml three-necked flask. After cooling to 0°, 140 mg of sodium hydride at 60% in oil, prewashed by settling out in toluene, are added portionwise and stirring is carried out at 0° for 1 hour. 160 μl of methyl iodide are then added and the mixture is allowed to return to ambient temperature for 20 hours with stirring. The pyridine is evaporated off under reduced pressure and the residue is taken up in 35 ml of dichloromethane and 10 ml of water. The organic phase is washed with water, dried over magnesium sulfate and concentrated under reduced pressure. After purifying by flash chromatography on silica gel (60; 30-75 μm), elution being carried out with a mixture of dichloromethane and ethanol (95-5 by volume), and then recrystallization from 25 ml of diethyl ether, 420 mg of [4-(3-dimethoxyphenyl)piperazin-1-yl](1-methyl-2-phenyl-1H-pyrrol-3-yl)methanone are obtained in the form of white crystals, the characteristics of which are as follows:

Mass spectrum (EI): m/z=405 (M⁺)

Melting point (Kofler bench)=130°.

### EXAMPLE 28

[4-(3,5-Dimethoxyphenyl)piperazin-1-yl](2-hydroxy-5-phenyl-1H-imidazol-4-yl)methanone

The reaction is carried out as in example 5 but from 150 mg of 2-hydroxy-5-phenyl-1-H-imidazol-4-ylcarboxylic acid, which can be prepared according to Heterocycles (1984), 22(8), 1763-9, and from 180 mg of 1-(3,5-dimethoxyphenyl)piperazine in 25 ml of dichloromethane, in the presence of 155 mg of 1-(3-dimethylaminopropyl)-3-ethylcarbodiimide hydrochloride (EDCl) and 10 mg of 1-hydroxybenzotriazole hydrate (HOBT), with stirring at ambient temperature for 24 hours. After purifying by flash chromatography on silica gel (60; 30-75 μm), elution being carried out with a mixture of dichloromethane and methanol (95-5 by volume), and then recrystallization from 15 ml of diethyl ether, 260 mg of 4-(3,5-dimethoxyphenyl)piperazin-1-yl](2-hydroxy-5-phenyl-1H-imidazol-4-yl)methanone are obtained in the form of white crystals, the characteristics of which are as follows:

Mass spectrum (EI): m/z=408 (M⁺)

Melting point (Kofler bench)=130°.

### EXAMPLE 29

[4-(3-Methoxyphenyl)piperazin-1-yl](4-phenyl-1H-imidazol-5-yl)methanone

The reaction is carried out as in example 5 but, on the one hand, from 188 mg of 4-phenyl-1H-imidazol-5-ylcarboxylic acid, obtained in stage 1 of example 3, and from 192 mg of 1-(3-methoxyphenyl)piperazine in 20 ml of dichloromethane, in the presence of 211 mg of 1-(3-dimethylaminopropyl)-3-ethylcarbodiimide hydrochloride (EDCl) and 148 mg of 1-hydroxybenzotriazole hydrate (HOBT), with stirring at ambient temperature for 20 hours. After purifying by flash chromatography on silica gel (60; 30-75 μm), elution being carried out with pure ethyl acetate, and then crystallization from a mixture of ethyl acetate and diisopropyl ether (10/90 by volume), 130 mg of [4-(3-methoxyphenyl)piperazin-1-yl](4-phenyl-1H-imidazol-5-yl)methanone are obtained in the form of a beige solid, the characteristics of which are as follows:

Mass spectrum (EI): m/z=362 (M+)

¹H NMR spectrum (300 MHz, (CD₃)₂SO, 6 in ppm): from 2.80 to 3.80 (series of large unresolved peaks: 8H in total); 3.72 (s: 3H); 6.40 (broad dd, J=8 and 1.5 Hz: 1H); 6.45 (broad s: 1H); 6.51 (broad d, J=8 Hz: 1H); 7.13 (t, J=8 Hz: 1H); 7.30 (broad t, J=7.5 Hz: 1H); 7.42 (broad t, J=7.5 Hz: 2H); 7.62 (broad d, J=7.5 Hz: 2H); 7.82 (s: 1H).

### EXAMPLE 30

[4-(3-Difluoromethoxyphenyl)piperazin-1-yl](5-phenyl-1H-imidazol-4-yl)methanone

Stage 1: tert-Butyl 4-(3-difluoromethoxyphenyl)piperazin-1-ylcarboxylate

A mixture of commercially available 1-boc piperazine (500.1 mg, 2.685 mmol) and commercially available 3-difluoromethoxybromobenzene (598.8 mg, 2.685 mmol) in toluene (20 ml) is placed in a 50 ml three-necked round-bottomed flask made inert with argon, and then the ligand (R)-(+)-2,2′-bis(diphenylphosphino)-1,1′-binaphthyl (56.850 mg, 91.2 μmol) and palladium(II)acetate (20.4 mg, 91.2 μmol) are added. The reaction mixture is stirred and brought to reflux for 16 hours. After returning to 20° C., the reaction mixture is diluted with water (20 ml) and then extracted with ethyl acetate (2×30 ml). The organic extracts are combined, dried over magnesium sulfate, filtered and evaporated under reduced pressure. The compound obtained is purified by chromatography on silica gel (AIT cartridge, Ref. FC-25 Si-BP-SUP, 20-40 μm, eluent dichloromethane, flow rate 20 ml/min). The fractions containing the expected compound are combined and then evaporated under reduced pressure. The expected tert-butyl 4-(3-difluoromethoxyphenyl)piperazin-1-ylcarboxylate is isolated (253 mg), the characteristics of which are as follows:

LC/MS analysis: tᵣ=4.18 min, M+H⁺ 329.31

Stage 2: 1-(3-Difluoromethoxyphenyl)piperazine hydrochloride

A solution of tert-butyl 4-(3-difluoromethoxyphenyl)piperazin-1-carboxylate (253 mg, 3.8 mmol) in a mixture of dioxane (1016 μl) and hydrochloric acid (963 μl) is placed in a 10 ml round-bottomed flask. The reaction mixture is stirred at 20° C. for 48 hours. The solid formed is filtered off, washed (diisopropyl ether, 10 ml) and dried under reduced pressure. The 1-(3-difluoromethoxyphenyl)piperazine hydrochloride is isolated, identified and characterized (189 mg), and used as is in the following step.

Stage 3: The reaction is carried out as in example 5 but from 376 mg of 4-phenyl-1H-imidazol-5-ylcarboxylic acid, obtained in stage 1 of example 3, and from 602 mg of 1-(3-difluoromethoxyphenyl)piperazine dihydrochloride, in 50 ml of dichloromethane, in the presence of 0.618 ml of triethylamine, 422 mg of 1-(3-dimethylaminopropyl)-3-ethylcarbodiimide hydrochloride (EDCl) and 296 mg of 1-hydroxybenzotriazole hydrate (HOBT), with stirring at ambient temperature for 20 hours. After purifying by flash chromatography on silica gel (60; 30-75 μm), elution being carried out with a mixture of dichloromethane and methanol (95/5 by volume), and then precipitation from diisopropyl ether, 455 mg of [4-(3-difluoromethoxyphenyl)piperazin-1-yl](5-phenyl-1H-imidazol-4-yl)methanone are obtained in the form of an amorphous beige solid, the characteristics of which are as follows:

Mass spectrum (EI): m/z=398 (M+)

¹H NMR spectrum (300 MHz, (CD₃)₂SO, 6 in ppm): from 2.90 to 3.90 (series of broad unresolved peaks: 8H in total); 6.58 (broad dd, J=8 and 1.5 Hz: 1H); 6.68 (broad s: 1H); 6.80 (broad dd, J=8 and 1.5 Hz: 1H); 7.20 (t, J=75 Hz: 1H); 7.25 (t, J=8 Hz: 1H); 7.30 (broad t, J=7.5 Hz: 1H); 7.43 (broad t, J=7.5 Hz: 2H); 7.63 (broad d, J=7.5 Hz: 2H); 7.82 (s: 1H).

### EXAMPLE 31

[4-(3-Chlorophenyl)piperazin-1-yl][1-(2-dimethylaminoethyl)-4-phenyl-1H-pyrrol-3-yl]methanone hydrochloride

200 mg of [4-(3-chlorophenyl)piperazin-1-yl](4-phenyl-1H-pyrrol-3-yl)methanone, prepared in example 15, are dissolved in 6 ml of pyridine. After cooling to 0° C., 49.3 mg of sodium hydride at 60% in oil, prewashed by settling out in toluene, are added portionwise and the mixture is stirred at 0° C. for 15 minutes. 79 mg of (2-chloroethyl)dimethylamine hydrochloride are then added, and the mixture is heated at 60° C. for 3 hours and then stirred at ambient temperature for 20 hours. The pyridine is concentrated under reduced pressure and the residue is taken up in 50 ml of ethyl acetate and the organic phase is washed with 3 times 25 ml of water, dried over magnesium sulfate and concentrated under reduced pressure. After purifying by flash chromatography on silica gel (60; 30-75 μm), elution being carried out with a mixture of dichloromethane and methanol (95-5 by volume), and after acidification with 1 equivalent of 1N hydrochloric ether, 80 mg of [4-(3-chlorophenyl)piperazin-1-yl][1-(2-dimethylaminoethyl)-4-phenyl-1H-pyrrol-3-yl]methanone hydrochloride are obtained in the form of a light brown foam, the characteristics of which are as follows:

Mass spectrum (EI): m/z=436 (M+)

¹H NMR spectrum (300 MHz, (CD₃)₂SO, δ in ppm): from 2.60 to 3.10 (broad unresolved peak: 4H); 2.81 (broad s: 6H); from 3.20 to 3.70 (broad unresolved peak: 4H); 3.56 (mt: 2H); 4.37 (t, J=7 Hz: 2H); 6.82 (mt: 2H); 6.87 (broad s: 1H); from 7.10 to 7.25 (mt: 4H); 7.33 (mt: 4H).

### EXAMPLE 32

3-{3-[4-(3-Chlorophenyl)piperazin-1-ylcarbonyl]-4-phenylpyrrol-1-yl}-propionic acid

200 mg of [4-(3-chlorophenyl)piperazin-1-yl](4-phenyl-1H-pyrrol-3-yl)methanone, prepared in example 15, are dissolved in 10 ml of pyridine. After cooling to 0° C., 49 mg of sodium hydride at 60% in oil, prewashed by settling out in toluene, are added portionwise and the mixture is stirred at 0° C. for 15 minutes. 91 mg of 3-bromopropionic acid methyl ester are then added and the mixture is heated to 60° C. for 3 hours and stirred at ambient temperature for 20 hours. The pyridine is concentrated under reduced pressure, the residue is taken up in 50 ml of ethyl acetate and the organic phase is washed with three times 25 ml of water, dried over magnesium sulfate and concentrated under reduced pressure. After purifying by flash chromatography on silica gel (60; 30-75 μm), elution being carried out with a mixture of dichloromethane and methanol (99-1 by volume), and recovering the second fraction, 115 mg of 3-{3-[4-(3-chlorophenyl)piperazin-1-yl-carbonyl]-4-phenylpyrrol-1-yl}propionic acid are obtained in the form of an amorphous beige solid, the characteristics of which are as follows:

Mass spectrum (EI): m/z=437 (M+)

¹H NMR spectrum (300 MHz, (CD₃)₂SO, 6 in ppm): from 2.70 to 3.60 (series of unresolved peaks: 8H in total); 2.80 (t, J=7 Hz: 2H); 4.16 (t, J=7 Hz: 2H); 6.80 (mt: 2H); 6.86 (broad t, J=2 Hz: 1H); 7.06 (d, J=2 Hz: 1H); from 7.10 to 7.25 (mt: 1H); 7.13 (d, J=2 Hz: 1H); 7.20 (t, J=8 Hz: 1H); 7.33 (mt: 4H).

### EXAMPLE 33

[4-(3,5-Dimethoxyphenyl)piperazin-1-yl](2-methanesulfinyl-5-phenyl-1H-imidazol-4-yl)methanone

984 mg of meta-chloroperbenzoic acid (MCPBA), at a temperature in the region of 0° C., are added to a solution of 1.1 g of [4-(3,5-dimethoxyphenyl)piperazin-1-yl](2-methylsulfanyl-5-phenyl-1H-imidazol-4-yl)methanone, obtained in example 23, in 25 ml of dichloromethane, and the mixture is stirred at ambient temperature for 20 hours. After washing with an aqueous 10% sodium bicarbonate solution and with water, the organic phase is dried over magnesium sulfate, concentrated under reduced pressure and purified by flash chromatography on silica gel (60; 30-75 μm), elution being carried out with a mixture of dichloromethane and methanol (97.5/2.5 by volume). 275 mg of [4-(3,5-dimethoxyphenyl)piperazin-1-yl](2-methanesulfinyl-5-phenyl-1H-imidazol-4-yl)methanone are thus obtained, in a first fraction, in the form of an amorphous pink solid, the characteristics of which are as follows:

Mass spectrum (EI): m/z=454 (M+)

¹H NMR spectrum (300 MHz, (CD₃)₂SO, 6 in ppm): 2.94 (unresolved peak: 2H); 3.08 (s: 3H); 3.20 (unresolved peak: 2H); 3.46 (unresolved peak: 2H); 3.70 (s: 6H); 3.76 (unresolved peak: 2H); 6.00 (t, J=2 Hz: 1H); 6.06 (d, J=2 Hz: 2H); 7.37 (broad t, J=7.5 Hz: 1H); 7.47 (broad t, J=7.5 Hz: 2H); 7.67 (broad d, J=7.5 Hz: 2H); 13.95 (unresolved peak: 1H).

### EXAMPLE 34

Methyl 3-{3-[4-(3-chlorophenyl)piperazin-1-ylcarbonyl]-4-phenylpyrrol-1-yl}-propionate

The reaction is carried out as in example 32, but the first fraction is collected. After acidification of this elution fraction with 132 μl of 1M hydrochloric acid, 53 mg of methyl 3-{3-[4-(3-chlorophenyl)piperazin-1-carbonyl]-4-phenylpyrrol-1-yl}propionate hydrochloride are collected in the form of an amorphous beige solid, the characteristics of which are as follows:

Mass spectrum (EI): m/z=451 (M+)

¹H NMR spectrum (300 MHz, (CD₃)₂SO, 6 in ppm): from 2.60 to 3.10 and from 3.30 to 3.80 (respectively broad unresolved peak and unresolved peak: 8H in total); 2.92 (t, J=7 Hz: 2H); 3.64 (s: 3H); 4.20 (t, J=7 Hz: 2H); 6.80 (mt: 2H); 6.87 (t, J=2 Hz: 1H); 7.06 (d, J=2.5 Hz: 1H); 7.13 (d, J=2.5 Hz: 1H); from 7.15 to 7.25 (mt: 1H); 7.20 (t, J=8 Hz: 1H); 7.34 (mt: 4H).

### EXAMPLE 35

[4-(3,5-Dimethoxyphenyl)piperazin-1-yl](1-hydroxymethyl-4-phenyl-1H-pyrrol-3-yl)methanone

2.98 ml of formaldehyde in aqueous solution at 37% and 0.66 ml of 1N sodium hydroxide are added to a solution of 235 mg of [4-(3,5-dimethoxyphenyl)piperazin-1-yl](4-phenyl-1-H-pyrrol-3-yl)methanone, prepared in example 16, in 4 ml of ethanol. After stirring at ambient temperature for 20 hours, the reaction mixture is concentrated under reduced pressure and then taken up with 50 ml of water and extracted with 3 times 25 ml of ethyl acetate and then washed with 2 times 25 ml of water. The organic phase is dried over magnesium sulfate and concentrated under reduced pressure. The residue obtained is purified by flash chromatography on silica gel (60; 30-75 μm), elution being carried out with a mixture of dichloromethane and methanol (95/5 by volume). 145 mg of [4-(3,5-dimethoxyphenyl)piperazin-1-yl](1-hydroxymethyl-4-phenyl-1H-pyrrol-3-yl)methanone are thus obtained in the form of an amorphous white solid, the characteristics of which are as follows:

Mass spectrum (EI): m/z=421 (M+)

¹H NMR spectrum (300 MHz, (CD₃)₂SO, δ in ppm): 2.85 (unresolved peak: 4H); 3.46 (unresolved peak: 4H); 3.68 (s: 6H); 5.25 (s: 2H); 5.98 (s: 3H); 6.63 (unresolved peak: 1H); 7.10 (d, J=2 Hz: 1H); 7.17 (d, J=2 Hz: 1H); 7.20 (mt: 1H); 7.34 (mt: 4H).

### EXAMPLE 36

[4-(3,5-Dimethoxyphenyl)piperazin-1-yl][1-(2-hydroxyethyl)-4-phenyl-1H-pyrrol-3-yl]methanone

Stage 1: 391.5 mg of [4-(3,5-dimethoxyphenyl)piperazin-1-yl](4-phenyl-1H-pyrrol-3-yl)methanone, prepared in example 16, are dissolved in 10 ml of pyridine. After cooling to 0° C., 90 mg of sodium hydride at 60% in oil, prewashed by settling out in toluene, are added portionwise and the mixture is stirred at 0° C. for 30 minutes. 241 mg of (2-bromoethoxy)-tert-butyldimethylsilane are then added and the mixture is heated at 60° C. for 3 hours and then stirred at ambient temperature for 20 hours. The pyridine is concentrated under reduced pressure and the residue is taken up in 50 ml of water and then extracted with 3 times 25 ml of ethyl acetate. After drying over magnesium sulfate and concentrating under reduced pressure, and then purifying by flash chromatography on silica gel (60; 30-75 μm), elution being carried out with a mixture of dichloromethane and methanol (97.5-2.5 by volume), 400 mg of {1-[2-(tert-butyldimethylsilanyloxy)ethyl]-4-phenyl-1H-pyrrol-3-yl}[4-(3,5-dimethoxyphenyl)piperazin-1-yl]methanone are obtained in the form of an orange-colored oil, the characteristic of which is as follows:

Mass spectrum (EI): m/z=549 (M+)

Stage 2: 5.82 ml of a 1M tetra-N-butylammonium fluoride solution in tetrahydrofuran are added to a solution of 400 mg of {1-[2-(tert-butyldimethylsilanyloxy)ethyl]-4-phenyl-1H-pyrrol-3-yl}[4-(3,5-dimethoxyphenyl)piperazin-1-yl]methanone in 12 ml of tetrahydrofuran. After stirring at 20° C. for 20 hours, 50 ml of ethyl acetate are added and the mixture is washed with 3 times 25 ml of water, dried over magnesium sulfate and concentrated to dryness under reduced pressure. The residue obtained is purified by flash chromatography on silica gel (60; 30-75 μm), elution being carried out with a mixture of dichloromethane and methanol (99/1 by volume). 180 mg of [4-(3,5-dimethoxyphenyl)piperazin-1-yl][1-(2-hydroxyethyl)-4-phenyl-1H-pyrrol-3-yl]-methanone are thus added in the form of an amorphous yellow solid, the characteristics of which are as follows:

Mass spectrum (EI): m/z=435 (M+)

¹H NMR spectrum (300 MHz, (CD₃)₂SO, δ in ppm): 2.85 (unresolved peak: 4H); 3.47 (unresolved peak: 4H); 3.69 (s: 6H); 3.72 (mt: 2H); 3.99 (t, J=7.5 Hz: 2H); 4.96 (t, J=5 Hz: 1H); 5.98 (broad s: 3H); 7.04 (d, J=2 Hz: 1H); 7.09 (d, J=2 Hz: 1H); 7.16 (mt: 1H); 7.34 (mt: 4H).

### EXAMPLE 37

3-[4-(1-Methyl-4-phenyl-1H-pyrrol-3-yl-carbonyl)piperazin-1-yl]benzamide

The reaction is carried out as in example 5 but, on the one hand, 201 mg of 1-methyl-4-phenyl-1H-pyrrol-3-ylcarboxylic acid, which can be prepared according to reference Med. Chem. Res. (1997), 7(2), 98-108, and, on the other hand, from 278 mg of 1-(3-carboxyamidophenyl)piperazine dihydrochloride, which can be prepared according to WO 9800400, in 25 ml of dichloromethane, in the presence of 211 mg of 1-(3-dimethylaminopropyl)-3-ethylcarbodiimide hydrochloride (EDCl), and 148 mg of 1-hydroxybenzotriazole hydrate (HOBT), with stirring at ambient temperature for 20 hours. After purifying by flash chromatography on silica gel (60; 30-75 μm), elution being carried out with a mixture of dichloromethane and methanol (95/5 by volume), 240 mg of 3-[4-(1-methyl-4-phenyl-1H-pyrrol-3-yl-carbonyl)piperazin-1-yl]benzamide are obtained in the form of an amorphous white solid, the characteristics of which are as follows:

Mass spectrum (EI): m/z=388 (M+)

¹H mass spectrum (300 MHz, (CD₃)₂SO, δ in ppm): from 2.65 to 3.15 (broad unresolved peak: 4H); 3.50 (unresolved peak: 4H); 3.69 (s: 3H); 6.98 (mt: 1H); 7.01 (d, J=2 Hz: 1H); 7.04 (d, J=2 Hz: 1H); from 7.10 to 7.40 (mt: 9H); 7.86 (unresolved peak: 1H).

### EXAMPLE 38

[4-(2-Hydroxy-3,5-dimethoxyphenyl)piperazin-1-yl](2-methanesulfinyl-5-phenyl-1H-imidazol-4-yl)methanone

The reaction is carried out as in example 33, but the second eluted fraction is collected. 45 mg of [4-(2-hydroxy-3,5-dimethoxyphenyl)piperazin-1-yl](2-methanesulfinyl-5-phenyl-1H-imidazol-4-yl)methanone are thus obtained in the form of an amorphous purple solid, the characteristics of which are as follows:

Mass spectrum (EI): m/z=470 (M+)

¹H NMR spectrum (300 MHz, (CD₃)₂SO, 6 in ppm): 2.68 (unresolved peak: 2H); 2.97 (unresolved peak: 2H); 3.08 (s: 3H); 3.45 (unresolved peak: 2H); 3.68 (s: 3H); 3.75 (s: 3H); 3.79 (unresolved peak: 2H); 6.05 (broad d, J=2.5 Hz: 1H); 6.32 (d, J=2.5 Hz: 1H); 7.38 (broad t, J=7.5 Hz: 1H); 7.48 (broad t, J=7.5 Hz: 2H); 7.65 (broad d, J=7.5 Hz: 2H); 7.73 (broad s: 1H); 13.92 (broad unresolved peak: 1H).

### EXAMPLE 39

3-[4-(3-Phenyl-1H-pyrrol-2-ylcarbonyl)piperazin-1-yl]benzamide

Stage 1: A solution of 3.25 g of 1-boc-piperazine in 115 ml of toluene is placed in a 250 ml three-necked flask and then 369.4 mg of (R)-(+)-2,2′-bis(diphenylphosphino)-1,1′-binaphthyl, 3.176 g of 3-bromobenzonitrile, 133.2 mg of palladium acetate and 2.516 g of sodium tert-butoxide are added. The reaction mixture is stirred and heated at 80° C. for 16 hours and then diluted with 110 ml of water. The aqueous phase is separated by settling and is then extracted with 120 ml of ethyl acetate. The organic phases are combined, dried over magnesium sulfate, filtered and evaporated under reduced pressure. The compound obtained is purified by chromatography on silica gel (AIT cartridge, Ref. FC-150-Si-BP-SUP, 20-40 μm, loading solvent: dichloromethane, then elution with 75/25 v/v cyclohexane/ethyl acetate, flow rate of 20 ml/min until crystallization of the compound on the column). The silica column is cut up into 8 equal sections, the silica from each section then being extracted with ethyl acetate (20 ml), resulting in various fractions. The fractions containing the expected compound are combined and then evaporated under reduced pressure. 3.08 g of tert-butyl 4-(3-cyanophenyl)-piperazin-1-ylcarboxylate are thus obtained, the characteristics of which are as follows:

Infrared spectrum (KBr): 3070; 2979; 2223; 1684; 1599; 1373; 1489; 1427; 1393; 1368; 1364; 1243; 1160; 1126; 993; 953; 785 and 686 cm⁻¹

Mass spectrum: EI: m/z=287, M^{+.}; m/z=231, (M−C₄H₈)⁺; m/z=157 C₁₀H₉N₂⁺; m/z=57 C₄H₉⁺ base peak

Stage 2: A solution of 3.81 g of tert-butyl 4-(3-cyanophenyl)piperazin-1-yl-carboxylate, obtained above, in 100 ml of methanol, is placed in a 250 ml round-bottomed flask and 24 ml of a molar solution of aqueous sodium hydroxide are then added. The reaction mixture is refluxed for 36 hours and then evaporated under reduced pressure. The residue is taken up in 150 ml of ethyl acetate and 150 ml of water, and separated by settling. The aqueous phase is extracted with 100 ml of ethyl acetate. The organic extracts are combined, dried over magnesium sulfate, filtered and evaporated under reduced pressure, providing a compound which is taken up in a mixture of ethyl acetate (15 ml) and heptane (10 ml). The solid formed is filtered off, washed (1/1 ethyl acetate/heptane, 10 ml) and dried under reduced pressure. 2.01 g of tert-butyl 4-(3-carbamoylphenyl)piperazin-1-ylcarboxylate are thus isolated in the form of a beige solid, the characteristics of which are as follows:

¹H NMR spectrum (300 MHz, (CD₃)₂SO, 6 in ppm): 1.45 (s: 9H); 3.17 (mt: 4H); 3.48 (mt: 4H); 7.11 (dt, J=7.5 and 2 Hz: 1H); from 7.20 to 7.35 (mt: 3H); 7.44 (very broad s: 1H); 7.90 (unresolved peak: 1H).

Mass spectrum: EI: m/z=305 M^{+.}; m/z=249, (M−C₄H₈)⁺; m/z=163, C₉H₁₁N₂O⁺, base peak; m/z=57 C₄H₉⁺

Stage 3: A solution of 2.01 g of tert-butyl 4-(3-carbamoylphenyl)piperazin-1-ylcarboxylate in 8 ml of dioxane is placed in a 100 ml round-bottomed flask and then 8 ml of a 4M hydrochloric acid solution in dioxane are added and the mixture is stirred at 20° C. for 16 hours. The solid formed is filtered off, washed with ethyl ether and dried under reduced pressure. 1.57 g of 3-(piperazin-1-yl)benzamide are thus obtained, the characteristic of which is as follows:

LC/MS: Tr=1.48 min., M+H⁺ m/z 206.28.

Stage 4: A mixture of 500 mg of 3-phenyl-1H-pyrrol-2-ylcarboxylic acid, obtained in stage 1 of example 6, 563.2 mg of 1-(3-dimethylaminopropyl)-3-ethylcarbodiimide hydrochloride (EDCl), 397 mg of 1-hydroxybenzotriazole (HOBT) and 710.2 mg of 3-(piperazin-1-yl)benzamide with stirring in 40 ml of dichloromethane is placed in a 100 ml three-necked flask, placed under argon, and then 1.24 ml of triethylamine are added. The reaction mixture is stirred at 20° C. for 16 hours and then diluted with 50 ml of dichloromethane and 50 ml of water. After separation by settling out, extraction is performed with 20 ml of dichloromethane. The organic extracts are combined, dried over magnesium sulfate, filtered and evaporated under reduced pressure. The crude compound obtained is taken up in ethyl acetate (15 ml) and methanol (5 ml), dissolved, and left at 20° C. for 48 hours. The solid formed is filtered off, washed with ethyl acetate (5 ml) then with ethyl ether and dried under reduced pressure. 778 mg of actual product are thus obtained, of which 80 mg are recrystallized from a mixture of ethyl acetate (5 ml) and methanol (5 ml), filtered, washed with ethyl acetate (5 ml) and dried. 55 mg of 3-[4-(3-phenyl-1H-pyrrol-2-ylcarbonyl)piperazin-1-yl]benzamide are thus isolated, the characteristics of which are as follows:

¹H NMR spectrum (300 MHz, (CD₃)₂SO d6, δ in ppm): 2.92 (unresolved peak: 4H); 3.44 (unresolved peak: 4H); 6.34 (t, J=2.5 Hz: 1H); 6.94 (t, J=2.5 Hz: 1H); 6.98 (d mt, J=7.5 Hz: 1H); from 7.15 to 7.40 (mt: 9H); 7.86 (unresolved peak: 1H); 11.49 (unresolved peak: 1H).

Mass spectrum: EI: m/z=374 M^{+.}; m/z=212 C₁₃H₁₂N₂O⁺.; m/z=175 C₁₀H₁₁N₂O⁺ base peak; m/z=170C₁₁H₈NO⁺

Melting point: 2590 (Kofler bench)

### EXAMPLE 40

[4-(3,5-Dimethoxyphenyl)piperazin-1-yl](1-methyl-3-phenyl-1H-pyrrol-2-yl)-methanone hydrochloride

A solution of 99.9 mg of [4-(3,5-dimethoxyphenyl)piperazin-1-yl](3-phenyl-1H-pyrrol-2-yl)methanone, obtained in example 8, in 1 ml of dimethylformamide, is placed in a 5 ml Weathon reactor and then 38.8 mg of potassium carbonate and 17.5 μl of iodomethane are added. The reaction mixture is stirred at 20° C. overnight. Since the reaction is not yet finished, 20 mg of sodium hydride and a further 18 μl of iodomethane are then introduced and the reaction is continued at 20° C. for 60 minutes. The reaction mixture is diluted with water (15 ml) and then extracted with ethyl acetate (15 ml). The aqueous phase is extracted with ethyl acetate (2×10 ml). The organic extracts are combined, dried over magnesium sulfate, filtered and evaporated under reduced pressure. The compound obtained is purified by chromatography on silica gel (26×135 cartridge, Ref. 1511-1000, 10 g silica, 15-40 μm, eluent 9/1 v/v cyclohexane/ethyl acetate, flow rate of 10 ml/min). The fractions containing the expected compound are combined and then evaporated under reduced pressure, providing a compound which is triturated in ethyl ether (5 ml) for 15 hours. The solid formed is filtered off, washed with ethyl ether and dried under reduced pressure. 62.8 mg of [4-(3,5-dimethoxyphenyl)piperazin-1-yl](1-methyl-3-phenyl-1H-pyrrol-2-yl)methanone are thus isolated, (62.8 mg, 56%), the characteristics of which are as follows:

¹H NMR spectrum (300 MHz, (CD₃)₂SO, 6 in ppm): from 2.70 to 3.40 (several broad unresolved peaks: 4H in total); 3.61 (s: 3H); 3.69 (s: 6H); from 3.60 to 4.00 (unresolved peak: 4H); 5.98 (s: 3H); 6.31 (d, J=3 Hz: 1H); 6.94 (d, J=3 Hz: 1H); 7.20 (mt: 1H); from 7.25 to 7.40 (mt: 4H).

Mass spectrum: EI m/z=405 M⁺; m/z=192, ClH₁₄NO₂⁺, base peak; m/z=184, C₁₂H₁₀NO⁺

### EXAMPLE 41

1-{3-[4-(3,5-Dimethoxyphenyl)piperazin-1-yl-carbonyl]-4-phenylpyrrol-1-yl}-ethanone 98 μl of N,N-diisopropylethylamine (DIPEA), 62 mg of 4-dimethylaminopyridine (DMAP) and 40 μl of acetyl chloride are added to a solution of 200 mg of [4-(3,5-dimethoxyphenyl)piperazin-1-yl](4-phenyl-1-H-pyrrol-3-yl)-methanone, prepared in example 18, in 15 ml of dichloromethane. After stirring at ambient temperature for 20 hours, the reaction medium is taken up with 25 ml of water and 25 ml of dichloromethane and then washed once with 25 ml of water. The organic phase is dried over magnesium sulfate and concentrated under reduced pressure. The yellow oil obtained is purified by flash chromatography on silica gel (60; 30-75 μm), elution being carried out with a mixture of dichloromethane and methanol (98/2 by volume). 120 mg of 1-{3-[4-(3,5-dimethoxyphenyl)piperazin-1-yl-carbonyl]-4-phenylpyrrol-1-yl}-ethanone are thus obtained in the form of an amorphous beige solid, the characteristics of which are as follows:

Mass spectrum (EI): m/z=433 (M+)

¹H NMR spectrum (300 MHz, (CD₃)₂SO, 6 in ppm): 2.65 (s: 3H); 2.68 (unresolved peak: 2H); 3.10 (unresolved peak: 2H); from 3.20 to 3.35 (unresolved peak: 2H); 3.68 (unresolved peak: 2H); 3.68 (s: 6H); 6.00 (broad s: 3H); 7.30 (tt, J=7.5 and 1.5 Hz: 1H); 7.40 (broad t, J=7.5 Hz: 2H); 7.48 (broad d, J=7.5 Hz: 2H); 7.68 (d, J=2 Hz: 1H); 7.79 (d, J=2 Hz: 1H).

### EXAMPLE 42

(2-Amino-4-phenylthiazol-5-yl)[4-(3,5-dimethoxyphenyl)piperazin-1-yl]-methanone

Stage 1: 4 ml of a 1N aqueous sodium hydroxide solution and 10 ml of ethanol are added to a solution of 260 mg of ethyl 2-amino-4-phenylthiazol-5-yl-carboxylate, which can be prepared according to WO 03/024948. After 72 hours at ambient temperature, the reaction mixture is concentrated under reduced pressure and the residue is acidified with 1N hydrochloric acid until pH 1 is obtained. After filtering the solid formed, 210 mg of 2-amino-4-phenylthiazol-5-ylcarboxylic acid are thus obtained in the form of a white solid, the characteristic of which is as follows:

Mass spectrum (EI): m/z=220 (M+)

Stage 2: 192 mg of 1-(3-dimethylaminopropyl)-3-ethylcarbodiimide hydrochloride (EDCl) and 135 mg of 1-hydroxybenzotriazole hydrate (HOBT) are added to a solution of 200 mg of 2-amino-4-phenylthiazol-5-ylcarboxylic acid and 202 mg of 1-(3,5-dimethoxyphenyl)piperazine in 25 ml of dichloromethane. The mixture is stirred at ambient temperature for 20 hours and, after purifying by flash chromatography on silica gel (60; 30-75 μm), elution being carried out with a mixture of dichloromethane and methanol (97.5/2.5 by volume), and then solidifying from diisopropyl ether, 245 mg of 2-amino-4-phenylthiazol-5-yl)[4-(3,5-dimethoxyphenyl)piperazin-1-yl]-methanone are obtained in the form of an amorphous beige solid, the characteristics of which are as follows:

Mass spectrum (EI): m/z=424 (M+)

¹H NMR spectrum (300 MHz, (CD₃)₂SO, 6 in ppm): 2.84 (unresolved peak: 4H); 3.42 (unresolved peak: 4H); 3.68 (s: 6H); 5.98 (s: 3H); from 7.25 to 7.45 (mt: 5H); 7.56 (broad d, J=7.5 Hz: 2H).

### EXAMPLE 43

[4-(3,5-Dimethoxyphenyl)piperazin-1-yl](2-methyl-5-phenyl-3H-imidazol-4-yl)methanone

Stage 1:

1 g of potassium hydroxide pellets is added to a solution of 3.5 g of ethyl 2-methyl-5-phenyl-1H-imidazol-4-ylcarboxylate, which can be obtained according to patent application WO 95/04724, in 30 ml of distilled water and 60 ml of ethanol. After refluxing for 20 hours and then returning to ambient temperature, the reaction mixture is concentrated under reduced pressure and the residue is acidified with 1N hydrochloric acid. After filtering the solid formed, 3 g of 2-methyl-5-phenyl-1H-imidazol-4-ylcarboxylic acid are thus obtained in the form of a beige solid, the characteristic of which is as follows:

Mass spectrum (EI): m/z=202 (M+)

Stage 2: 146 mg of 1-(3-dimethylaminopropyl)-3-ethylcarbodiimide hydrochloride (EDCl), 103 mg of 1-hydroxybenzotriazole hydrate (HOBT) and 154 mg of 1-(3,5-dimethoxyphenyl)piperazine are added to a soluton of 140 mg of 2-methyl-5-phenyl-1H-imidazol-4-ylcarboxylic acid in 15 ml of dichloromethane and this reaction mixture is then stirred at ambient temperature for 20 hours. After adding 25 ml of dichloromethane and 25 ml of water, the organic phase is separated by settling and then washed with water, dried over magnesium sulfate and concentrated under reduced pressure. After purifying by flash chromatography on a column of silica (60; 35-70 μm), elution being carried out with a mixture of dichloromethane and methanol (95/5 by volume), 100 mg of [4-(3,5-dimethoxyphenyl)piperazin-1-yl]-(2-methyl-5-phenyl-3H-imidazol-4-yl)methanone are thus obtained in the form of an amorphous white solid, the characteristic of which is as follows:

Mass spectrum (EI): m/z=406 (M⁺)

### EXAMPLE 44

3-[4-(2-Mercapto-4-phenyl-1H-imidazol-5-ylcarbonyl)piperazin-1-yl]benzamide

The reaction is carried out as in example 5 but, on the one hand, from 440 mg of 2-mercapto-4-phenyl-1H-imidazol-5-ylcarboxylic acid, which can be prepared according to Chem. Pharm. Bull. (1984), 32(7), 2536-43, and, on the other hand, from 560 mg of 1-(3-carboxamidophenyl)piperazine dihydrochloride, which can be prepared according to WO 98/00400, in 75 ml of dichloromethane, in the presence of 421 mg of 1-(3-dimethylaminopropyl)-3-ethylcarbodiimide hydrochloride (EDCl), 297 mg of 1-hydroxybenzotriazole hydrate (HOBT) and 0.7 ml of triethylamine, with stirring at ambient temperature for 20 hours. After purifying with flash chromatography on silica gel (60; 30-75 μm), elution being carried out with a mixture of dichloromethane and methanol (95/5 by volume), 266 mg of 3-[4-(2-mercapto-4-phenyl-1H-imidazol-5-yl-carbonyl)piperazin-1-yl]benzamide are obtained in the form of an amorphous yellow solid, the characteristic of which is as follows:

Mass spectrum (EI): m/z=407 (M+)

### EXAMPLE 45

[4-(3,5-Dimethoxyphenyl)piperazin-1-yl][1-(thiazol-4-yl)methyl-4-phenyl-1H-pyrrol-3-yl]methanone

250 mg of [4-(3,5-dimethoxyphenyl)piperazin-1-yl](4-phenyl-1-H-pyrrol-3-yl)-methanone, prepared in example 16, are dissolved in 10 ml of pyridine. After cooling to 0° C., 59 mg of sodium hydride at 60% in oil, prewashed by settling out in toluene, are added portionwise and the mixture is stirred at 0° C. for 30 minutes. 108 mg of 4-chloromethylthiazole hydrochloride are then added and the mixture is heated at 60° C. for 6 hours and then stirred at ambient temperautre for 20 hours. The pyridine is concentrated under reduced pressure and the residue is taken up in 50 ml of water and then extracted with 3 times 25 ml of ethyl acetate. After drying over magnesium sulfate and concentrating under reduced pressure, and purifying by flash chromatography on silica gel (60; 30-75 μm), elution being carried out with a mixture of dichloromethane and methanol (98-2 by volume), then by crystallization from diisopropyl ether, 170 mg of [4-(3,5-dimethoxyphenyl)piperazin-1-yl]-[1-(thiazol-4-yl)methyl-4-phenyl-1H-pyrrol-3-yl]methanone are obtained in the form of an amorphous ochre-yellow solid, the characteristic of which is as follows:

Mass spectrum (EI): m/z=488 (M+)

### EXAMPLE 46

4-{3-[4-(3,5-Dimethoxyphenyl)piperazin-1-yl]carbonyl-4-phenyl-1H-pyrrol-1-yl}-butanoic acid

250 mg of [4-(3,5-dimethoxyphenyl)piperazin-1-yl](4-phenyl-1-H-pyrrol-3-yl)-methanone, prepared in example 16, are dissolved in 10 ml of pyridine. After cooling to 0° C., 59 mg of sodium hydride at 60% in oil, prewashed by settling out in toluene, are added portionwise and the mixture is stirred at 0° C. for 30 minutes. 96 μl of ethyl 4-bromobutanoate are then added and the mixture is heated at 60° C. for 8 hours and then stirred at ambient temperature for 20 hours. The pyridine is concentrated under reduced pressure and the residue is taken up in 50 ml of water and is then extracted with 3 times 25 ml of ethyl acetate. The aqueous phase is acidified to a pH of 4 by addition of 1N hydrochloric acid, and is then extracted 3 times with 25 ml of dichloromethane. The combined “dichloromethane” phases are concentrated to dryness under reduced pressure and the residue is crystallized from diisoproyl ether. 142 mg of 4-{3-[4-(3,5-dimethoxyphenyl)piperazin-1-yl]carbonyl-4-phenyl-1H-pyrrol-1-yl}butanoic acid are thus obtained in the form of an amorphous yellow solid, the characteristic of which is as follows:

Mass spectrum (EI): m/z=477 (M+)

### EXAMPLE 47

2-{3-[4-(3,5-Dimethoxyphenyl)piperazin-1-yl]carbonyl-4-phenyl-1H-pyrrol-1-yl}acetic acid

250 mg of [4-(3,5-dimethoxyphenyl)piperazin-1-yl](4-phenyl-1H-pyrrol-3-yl)-methanone, prepared in example 16, are dissolved in 10 ml of pyridine. After cooling to 0° C., 59 mg of sodium hydride at 60% in oil, prewashed by settling out in toluene, are added portionwise and the mixture is stirred at 0° C. for 30 minutes. 60.5 μl of ethyl bromoacetate are then added and the mixture is heated at 60° C. for 6 hours and then stirred at ambient temperature for 20 hours. The pyridine is concentrated under reduced pressure and the residue is taken up in 50 ml of water and is then extracted with 3 times 25 ml of ethyl acetate. The aqueous phase is acidified to a pH of 4 by addition of 1N hydrochloric acid and is then extracted 3 times with 25 ml of dichloromethane. The combined “dichloromethane” phases are concentrated to dryness under reduced pressure and the residue is purified by flash chromatography on silica gel (60; 30-75 μm), elution being carried out with a mixture of dichloromethane and methanol (95-5 by volume). 42 mg of 2-{3-[4-(3,5-dimethoxyphenyl)piperazin-1-yl]carbonyl-4-phenyl-1H-pyrrol-1-yl}acetic acid are thus obtained in the form of an amorphous orange solid, the characterstic of which is as follows:

Mass spectrum (EI): m/z=449 (M+)

### EXAMPLE 48

[4-(3,5-Dimethoxyphenyl)piperazin-1-yl][1-(pyridin-3-yl)methyl-4-phenyl-1H-pyrrol-3-yl]methanone

250 mg of [4-(3,5-dimethoxyphenyl)piperazin-1-yl](4-phenyl-1-H-pyrrol-3-yl)-methanone, prepared in example 16, are dissolved in 10 ml of pyridine. After cooling to 0° C., 57 mg of sodium hydride at 60% in oil, prewashed by settling out out in toluene, are added portionwise and the mixture is stirred at 0° C. for 30 minutes. 161.6 mg of 3-bromomethylpyridine hydrochloride are then added and the mixture is heated at 60° C. for 6 hours and then stirred at ambient temperature for 20 hours. The pyridine is concentrated under reduced pressure and the residue is then taken up in 50 ml of water, then extracted with 3 times 25 ml of ethyl acetate. After drying over magnesium sulfate and concentrating under reduced pressure, and purifying by flash chromatography on silica gel (60; 30-75 μm), elution being carried out with a mixture of dichloromethane and methanol (98-2 by volume), and then by crystallization from diisopropyl ether, 75 mg of [4-(3,5-dimethoxyphenyl)piperazin-1-yl]-[1-(pyridin-3-yl)methyl-4-phenyl-1H-pyrrol-3-yl]methanone are obtained in the form of an amorphous pale yellow solid, the characteristic of which is as follows:

Mass spectrum (EI): m/z=482 (M+)

### EXAMPLE 49

Methyl 2-{3-[4-(3,5-dimethoxyphenyl)piperazin-1-yl]carbonyl-2-phenyl-1H-pyrrol-1-yl}acetate

350 mg of [4-(3,5-dimethoxyphenyl)piperazin-1-yl](2-phenyl-1-H-pyrrol-3-yl)methanone, prepared in example 8, are dissolved in 15 ml of pyridine. After cooling to 0° C., 54 mg of sodium hydride at 60% in oil, prewashed by settling out in toluene, are added portionwise and the mixture is stirred at 0° C. for 30 minutes. 90 μl of methyl bromoacetate are then added and the mixture is stirred at ambient temperature for 20 hours. The pyridine is concentrated under reduced pressure and the residue is taken up in 50 ml of water and then extracted with 3 times 25 ml of ethyl acetate. The combined organic phases are washed with water, dried over magnesium sulfate and concentrated to dryness under reduced pressure. The residue is purified by flash chromatography on silica gel (60; 30-75 μm), elution being carried out with a mixture of dichloromethane and methanol (97.5-2,5 by volume). 230 mg of methyl 2-{3-[4-(3,5-dimethoxyphenyl)piperazin-1-yl]carbonyl-2-phenyl-1H-pyrrol-1-yl}acetate are thus obtained in the form of an amorphous orange solid, the characteristic of which is as follows:

Mass spectrum (EI): m/z=463 (M+)

### EXAMPLE 50

3-[4-(1-Methyl-2-phenyl-1H-pyrrol-3-ylcarbonyl)piperazin-1-yl]benzamide

The reaction is carried out as in example 5 but, on the one hand, from 430 mg of 1-methyl-2-phenyl-1H-pyrrol-3-ylcarboxylic acid, which can be prepared as in stage 1 of example 21, and, on the other hand, from. 420 mg of 1-(3-carboxamidophenyl)piperazine dihydrochloride, which can be prepared according to WO 98/00400, in 50 ml of dichloromethane, in the presence of 320 mg of 1-(3-dimethylaminopropyl)-3-ethylcarbodiimide hydrochloride (EDCl), 20 mg of 1-hydroxybenzotriazole hydrate (HOBT) and 0.6 ml of triethylamine, with stirring at ambient temperature for 20 hours. After purifying by crystallization from 13 ml of diisopropyl ether, 260 mg of 3-[4-(1-methyl-2-phenyl-1H-pyrrol-3-yl-carbonyl)piperazin-1-yl]benzamide are obtained in the form of white crystals, the characteristics of which are as follows:

Mass spectrum (EI): m/z=407 (M⁺)

Melting point (Kofler bench)=172° C.

### EXAMPLE 51

3-[4-(2-Hydroxy-4-phenyl-1H-imidazol-5-yl-carbonyl)piperazin-1-yl]benzamide

The reaction is carried out as in example 5 but, on the one hand from 410 mg of 2-hydroxy-4-phenyl-1H-imidazol-5-ylcarboxylic acid, which can be prepared according to Heterocycles 1984, 22(8), 1763-69, and, on the other hand, from 610 mg of 1-(3-carboxamidophenyl)piperazine dihydrochloride, which can be prepared according to WO 98/00400, in 50 ml of dichloromethane, in the presence of 420 mg of 1-(3-dimethylaminopropyl)-3-ethylcarbodiimide hydrochloride (EDCl), 27 mg of 1-hydroxybenzotriazole hydrate (HOBT) and 0.82 ml of triethylamine, with stirring at ambient temperature for 48 hours. After purifying by crystallization from 20 ml of 1,2 dimethoxyethane, 500 mg of 3-[4-(2-hydroxy-4-phenyl-1H-imidazol-5-yl-carbonyl)piperazin-1-yl]benzamide are obtained in the form of white crystals, the characteristics of which are as follows:

Mass spectrum (EI): m/z=391 (M+)

Melting point (Kofler bench)=202° C.

### EXAMPLE 52

3-[4-(2-Mercapto-4-phenyl-1H-imidazol-5-yl-carbonyl)piperazin-1-yl]-benzonitrile

The reaction is carried out as in example 5 but, on the one hand, from 300 mg of 2-mercapto-4-phenyl-1H-imidazol-5-yl-carboxylic acid, which can be prepared according to Chem. Pharm. Bull. (1984), 32(7), 2536-43, and, on the other hand, from 354 mg of 1-(3-cyanophenyl)piperazine dihydrochloride, which can be prepared according to Tetrahedron Lett (2000), 56(24), 4107-10, in 50 ml of dichloromethane, in the presence of 287 mg of 1-(3-dimethylaminopropyl)-3-ethylcarbodiimide hydrochloride (EDCl), 202 mg of 1-hydroxybenzotriazole hydrate (HOBT) and 0.57 ml of triethylamine, with stirring at ambient temperature for 20 hours. After purifying by flash chromatography on silica gel (60; 30-75 μm), elution being carried out with a mixture of dichloromethane and methanol (99/1 by volume), 431 mg of 3-[4-(2-mercapto-4-phenyl-1H-imidazol-5-ylcarbonyl)piperazin-1-yl]benzamide are obtained in the form of an amorphous white solid, the characteristics of which are as follows:

Mass spectrum (EI): m/z=389 (M+)

Melting point (Kofler bench)=247° C.

¹H NMR spectrum (400 MHz)—δ in ppm—in d6-DMSO: from 2.83 to 3.80 (very broad m, 8H); 7.19 (td, J=1.0 and 7.5 Hz, 1H); 7.23 (ddd, J=1.0-2.5 and 7.5 Hz, 1H); 7.31 (dd, J=1.0 and 2.5 Hz, 1H); from 7.35 to 7.42 (m, 2H); 7.44 (broad t, J=7.5 Hz, 2H); 7.51 (broad d, J=7.5 Hz, 2H); 12.6 (broad m, 1H); 12.8 (broad, 1H).

### EXAMPLE 53

[4-(3,5-Dimethoxyphenyl)piperazin-1-yl][1-(2-hydroxyethyl)-2-phenyl-1H-pyrrol-3-yl]methanone

Stage 1: 391.5 mg of [4-(3,5-dimethoxyphenyl)piperazin-1-yl](2-phenyl-1H-pyrrol-3-yl)methanone, prepared in example 8, are dissolved in 20 ml of pyridine. After cooling to 0° C., 64.5 mg of sodium hydride at 60% in oil, prewashed by settling out in toluene, are added portionwise and the mixture is stirred at 0° C. for 30 minutes. 0.25 ml of (2-bromoethoxy)-tert-butyldimethylsilane is then added and the mixture is stirred at ambient temperature for 20 hours. The pyridine is concentrated under reduced pressure and the residue is taken up in 50 ml of water and is then extracted with 3 times 25 ml of ethyl acetate. After drying over magnesium sulfate and concentrating under reduced pressure, 570 mg of {1-[2-(tert-butyldimethylsilanyloxy)ethyl]-2-phenyl-1H-pyrrol-3-yl}[4-(3,5-dimethoxyphenyl)piperazin-1-yl]methanone are obtained in the form of an orange-colored oil used as is in the following step, the characteristic of which is as follows:

Mass spectrum (EI): m/z=549 (M+)

Stage 2: 8 ml of a 1M tetra-N-butylammonium fluoride solution in tetrahydrofuran are added to a solution of 550 mg of {1-[2-(tert-butyidimethylsilanyloxy)ethyl]-2-phenyl-1H-pyrrol-3-yl}[4-(3,5-dimethoxyphenyl)piperazin-1-yl]methanone in 20 ml of tetrahydrofuran. After stirring for 20 hours at 20° C., 50 ml of ethyl acetate are added and the product is washed with 3 times 25 ml of water, dried over magnesium sulfate and concentrated to dryness under reduced pressure. The residue obtained is purified by flash chromatography on silica gel (60; 30-75 μm), elution being carried out with a mixture of dichloromethane and ethanol (95/5 by volume). After recrystallizing from 15 ml of diethyl ether, 240 mg of [4-(3,5-dimethoxyphenyl)piperazin-1-yl][1-(2-hydroxyethyl)-2-phenyl-1H-pyrrol-3-yl]methanone are thus obtained in the form of white crystals, the characteristics of which are as follows:

Mass spectrum (EI): m/z=435 (M+)

Melting point (Kofler bench)=157° C.

### EXAMPLE 54

3-[4-(2-Trifluoromethyl-4-phenyl-1H-imidazol-5-ylcarbonyl)piperazin-1-yl]-benzamide

Stage 1: 200 mg of ethyl 4-phenyl-2-trifluoromethyl-1-H-imidazol-2-carboxylate, which can be prepared according to WO 95/04724, are dissolved in 10 ml of tetrahydrofuran. 185 mg of lithium hydroxide monohydrate are then added and the mixture is stirred for 20 hours at ambient temperature. After concentrating under reduced pressure, the residue is dissolved in 5 ml of water and a 1N hydrochloric acid solution is added until a pH of 6 is obtained. The precipitate formed is filtered off and dried under vacuum, and 160 mg of 4-phenyl-2-trifluoromethyl-1-H-imidazol-2-ylcarboxylic acid are thus obtained in the form of a white solid used as is in the following stage.

Stage 2: The reaction is carried out as in example 5 but, on the one hand, from 120 mg of 2-trifluoromethyl-4-phenyl-1H-imidazol-5-ylcarboxylic acid and, on the other hand, from 130 mg of 1-(3-carboxamidophenyl)piperazine dihydrochloride, which can be prepared according to WO 98/00400, in 20 ml of dichloromethane, in the presence of 99 mg of 1-(3-dimethylaminopropyl)-3-ethylcarbodiimide hydrochloride (EDCl), 70 mg of 1-hydroxybenzotriazole hydrate (HOBT) and 0.20 ml of triethylamine, with stirring at ambient temperature for 20 hours. After purifying by flash chromatography on silica gel (60; 30-75 μm), elution being carried out with a gradient of mixtures of dichloromethane and ethanol (from 99/1 to 95/5 by volume), 79 mg of 3-[4-(2-trifluoromethyl-4-phenyl-1H-imidazol-5-ylcarbonyl)piperazin-1-yl]benzamide are obtained in the form of a white foam, the characteristic of which is as follows:

Mass spectrum (EI): m/z=443 (M+)

### EXAMPLE 55

3-[4-(2-Methylsulfanyl-4-phenyl-1H-imidazol-5-ylcarbonyl)piperazin-1-yl]-benzamide

150 mg of 3-[4-(2-mercapto-4-phenyl-1H-imidazol-5-ylcarbonyl)piperazin-1-yl]benzamide, obtained in example 44, are suspended in 13 ml of methanol, then 24 mg of sodium methoxide are added, and the mixture is stirred at ambient temperature for 20 minutes until complete dissolution. 25 μl of methyl iodide are then added and the mixture is heated at 40° C. for 1 hour and 45 minutes. The methanol is then concentrated under reduced pressure and the residue is taken up with a mixture of water and dichloromethane. The aqueous phase is re-extracted with dichloromethane. The combined organic phases are washed with water, dried over magnesium sulfate and concentrated to dryness. After recrystallizing from diisopropyl ether, 153 mg of 3-[4-(2-methylsulfanyl-4-phenyl-1H-imidazol-5-ylcarbonyl)piperazin-1-yl]-benzamide are thus obtained in the form of a yellow powder, the characteristic of which is as follows:

Mass spectrum (EI): m/z=421 (M+)

### EXAMPLE 56

3-[4-(2-Methylsulfanyl-4-phenyl-1H-imidazol-5-ylcarbonyl)piperazin-1-yl]-benzonitrile

The reaction is carried out as in example 55 but from 200 mg of 3-[4-(2-mercapto-4-phenyl-1H-imidazol-5-ylcarbonyl)piperazin-1-yl]benzonitrile, obtained in example 52, 33 mg of sodium methoxide and 35 μl of methyl iodide in 20 ml of methanol. After recrystallizing from diisopropyl ether, 106 mg of 3-[4-(2-methylsulfanyl-4-phenyl-1H-imidazol-5-ylcarbonyl)piperazin-1-yl]benzonitrile are thus obtained in the form of a white powder, the characteristic of which is as follows:

Mass spectrum (EI): m/z=403 (M+)

¹H NMR spectrum (300 MHz)—δ in ppm—in d6-DMSO: 2.61 (s, 3H); from 3.03 to 3.37 (very broad m, 4H); from 3.46 to 3.80 (very broad m, 4H); 7.19 (broad d, J=8.0 Hz, 1H); from 7.22 to 7.44 (m, 6H); 7.58 (broad d, J=8.0 Hz, 2H); 12.8 (broad m, 1H).

### EXAMPLE 57

[4-(3-Hydroxymethylphenyl)piperazin-1-yl](2-hydroxy-4-phenyl-1H-imidazol-5-yl)methanone

Stage 1: 850 mg of 4-tert-butylcarbonyloxy-1-(3-hydroxymethylphenyl)piperazine, which can be prepared according to WO 00/15646, are dissolved in 40 ml of dioxane. 3.64 ml of a 4N hydrochloric acid solution in dioxane are then added and the mixture is stirred for 1 hour at 0° C. The precipitate formed is filtered off, washed with diethyl ether and dried under reduced pressure. 770 mg of 1-(3-hydroxymethylphenyl)piperazine dihydrochloride are thus obtained in the form of a yellow powder used as is in the following stage.

Stage 2: The reaction is carried out as in example 5 but, on the one hand, from 145 mg of 2-hydroxy-4-phenyl-1H-imidazol-5-ylcarboxylic acid, which can be prepared according to Heterocycles 1984, 22(8), 1763-6998-108, and, on the other hand, from 188 mg of 1-(3-hydroxymethylphenyl)piperazine dihydrochloride, in 25 ml of dichloromethane, in the presence of 150 mg of 1-(3-dimethylaminopropyl)-3-ethylcarbodiimide hydrochloride (EDCl), 105 mg of 1-hydroxybenzotriazole hydrate (HOBT) and 0.22 ml of triethylamine, with stirring at ambient temperature for 20 hours. After purifying by flash chromatography on silica gel (60; 30-75 μm), elution being carried out with a mixture of dichloromethane and ethanol (90/10 by volume), then crystallization from 5 ml of diethyl ether, 145 mg of [4-(3-hydroxymethylphenyl)piperazin-1-yl](2-hydroxy-4-phenyl-1H-imidazol-5-yl)methanone are obtained in the form of white crystals, the characteristics of which are as follows:

Mass spectrum (EI): m/z=378 (M+)

Melting point (Kofler bench)=173° C.

¹H NMR spectrum (400 MHz)—δ in ppm—in d6-DMSO: 3.10 (broad m, 4H); 3.62 (broad m, 4H); 4.44 (d, J=5.0 Hz, 2H); 5.09 (broad t, J=5.0 Hz, 1H); 6.79 (m, 2H); 6.90 (t, J=2.0, 1H); 7.18 (t, J=8.0 Hz, 1H); 7.21 (s, 2H); 7.36 (broad t, J=8.0 Hz, 1H); 7.42 (broad t, J=8.0 Hz, 2H); 7.75 (broad d, J=8.0 Hz, 1H).

### EXAMPLE 58

3-[4-(4-Phenyl-1H-pyrrol-3-ylcarbonyl)piperazin-1-yl]benzamide

The reaction is carried out as in example 5 but, on the one hand, from 1.404 g of 4-phenyl-1-H-pyrrol-3-ylcarboxylic acid, which can be prepared according to Med. Chem. Res. 1997, 7(2), 98-108, and, on the other hand, from 2.086 g of 1-(3-carboxamidophenyl)piperazine dihydrochloride, which can be prepared according to WO 98/00400, in 100 ml of dichloromethane, in the presence of 1.582 g of 1-(3-dimethylaminopropyl)-3-ethylcarbodiimide hydrochloride (EDCl), 1.115 g of 1-hydroxybenzotriazole hydrate (HOBT) and 2.32 ml of triethylamine, with stirring at ambient temperature for 20 hours. After purifying by flash chromatography on silica gel (60; 30-75 μm), elution being carried out with a gradient of mixtures of dichloromethane and methanol (from 95/5 to 90/10 by volume), 1.70 g of 3-[4-(4-phenyl-1H-pyrrol-3-ylcarbonyl)piperazin-1-yl]benzamide are obtained in the form of a beige powder, the characteristic of which is as follows:

Mass spectrum (EI): m/z=374 (M+)

### EXAMPLE 59

3-[4-(2-Methyl-5-phenyl-1H-imidazol-4-ylcarbonyl)piperazin-1-yl]benzamide

422 mg of 1-(3-dimethylaminopropyl)-3-ethylcarbodiimide hydrochloride (EDCl) and 297 mg 1-hydroxybenzotriazole hydrate (HOBT) are added to a solution of 404 mg of 2-methyl-5-phenyl-1H-imidazol-4-ylcarboxylic acid in 50 ml of dichloromethane. After stirring for 10 minutes at ambient temperature, 0.85 ml of triethylamine (TEA) and 556 mg of 3-piperazin-1-ylbenzamide dihydrochloride, which can be obtained according to patent application WO 98/00400, are added and this reaction mixture is then stirred at ambient temperature for 20 hours. After adding 50 ml of dichloromethane and 50 ml of water, the organic phase is separated by settling, then washed with water, dried over magnesium sulfate and concentrated under reduced pressure. After purifying by flash chromatography on a column of silica (60; 35-70 μm), elution being carried out with a mixture of dichloromethane and methanol (90/10 by volume), 425 mg of 3-[4-(2-methyl-5-phenyl-1H-imidazol-4-ylcarbonyl)piperazin-1-yl]benzamide are obtained in the form of an amorphous white solid, the characteristic of which is as follows:

Mass spectrum (EI): m/z=389 (M⁺)

### EXAMPLE 60

3-[4-(2-Methyl-5-phenyl-1H-imidazol-4-ylcarbonyl)piperazin-1-yl]benzonitrile

422 mg of 1-(3-dimethylaminopropyl)-3-ethylcarbodiimide hydrochloride (EDCl) and 297 mg of 1-hydroxybenzotriazole hydrate (HOBT) are added to a solution of 404 mg of 2-methyl-5-phenyl-1H-imidazol-4-ylcarboxylic acid in 50 ml of dichloromethane. After stirring at ambient temperature for 10 minutes, 0.62 ml of triethylamine (TEA) and 520 mg of 3-piperazin-1-ylbenzonitrile dihydrochloride, which can be obtained according to patent application WO 99/31096, are added and this reaction mixture is then stirred at ambient temperature for 20 hours. After adding 50 ml of dichloromethane and 50 ml of water, the organic phase is separated by settling, then washed with water, dried over magnesium sulfate and concentrated under reduced pressure. After purifying by flash chromatography on a column of silica (60; 35-70 μm), elution being carried out with a mixture of dichloromethane and methanol (95/05 by volume), 585 mg of 3-[4-(2-methyl-5-phenyl-1H-imidazol-4-ylcarbonyl)piperazin-1-yl]benzonitrile are obtained in the form of an amorphous white solid, the characteristic of which is as follows:

Mass spectrum (EI): m/z=371 (M⁺)

### EXAMPLE 61

3-[4-(4-Phenyl-1H-pyrrol-3-ylcarbonyl)piperazin-1-yl]benzonitrile

The reaction is carried out as in example 5 but, on the one hand, from 375 mg of 2-hydroxy-4-phenyl-1H-imidazol-5-ylcarboxylic acid, which can be prepared according to Med. Chem. Res. 1997, 7(2), 98-108 and, on the other hand, from 520 mg of 1-(3-cyanophenyl)piperazine dihydrochloride, which can be prepared according to Tetrahedron Lett. 2000, 56(24), 4107-10, in 50 ml of dichloromethane, in the presence of 422 mg of 1-(3-dimethylaminopropyl)-3-ethylcarbodiimide hydrochloride (EDCl), 297 mg of 1-hydroxybenzotriazole hydrate (HOBT) and 0.62 ml of triethylamine, with stirring at ambient temperature for 20 hours. After purifying by flash chromatography on silica gel (60; 30-75 μm), elution being carried out with a mixture of dichloromethane and methanol (95/5 by volume), 555 mg of 3-[4-(4-phenyl-1H-pyrrol-3-ylcarbonyl)piperazin-1-yl]benzonitrile are obtained in the form of an off-white powder, the characteristic of which is as follows:

Mass spectrum (EI): m/z=356 (M+)

### EXAMPLE 62

[4-(3,5-Dimethoxyphenyl)piperazin-1-yl](2-trifluoromethyl-4-phenyl-1H-imidazol-5-yl)methanone

The reaction is carried out as in example 5 but, on the one hand, from 200 mg of 2-trifluoromethyl-4-phenyl-1H-imidazol-5-ylcarboxylic acid, prepared as in stage 1 of example 54, and, on the other hand, from 174 mg of (3,5-dimethoxyphenyl)piperazine, in 30 ml of dichloromethane, in the presence of 165 mg of 1-(3-dimethylaminopropyl)-3-ethylcarbodiimide hydrochloride (EDCl) and 116 mg of 1-hydroxybenzotriazole hydrate (HOBT), with stirring at ambient temperature for 72 hours. After purifying by flash chromatography on silica gel (60; 30-75 μm), eluting with a gradient of mixtures of dichloromethane and methanol (from 100/0 to 99.5/0.5 by volume) and then recrystallization from 5 ml of diethyl ether, 87 mg of [4-(3,5-dimethoxyphenyl)piperazin-1-yl](2-trifluoromethyl-4-phenyl-1H-imidazol-5-yl)-methanone are obtained in the form of white crystals, the characteristics of which are as follows:

Mass spectrum (EI): m/z=460 (M+)

¹H NMR spectrum (400 MHz)—δ in ppm—in d6-DMSO at 373K: 3.10 (broad m, 4H); 3.62 (broad m, 4H); 3.73 (m, 6H); 6.01 (t, J=2.0 Hz, 1H); 6.06 (d, J=2.0 Hz, 2H); 7.38 (broad t, J=8.0 Hz, 1H); 7.46 (broad t, J=8.0 Hz, 2H); 7.68 (broad d, J=8.0 Hz, 1H); 13.9 (broad m, 1H).

### EXAMPLE 63

[4-(3,5-Dimethoxyphenyl)piperazin-1-yl](1-acetyl-2-phenyl-1H-pyrrol-3-yl)methanone

300 mg of [4-(3,5-dimethoxyphenyl)piperazin-1-yl](2-phenyl-1H-pyrrol-3-yl)methanone, obtained in example 21, are dissolved in 15 ml of pyridine. After cooling to 0° C., 46 mg of sodium hydride at 60% in oil, prewashed by settling out in toluene, are added portionwise and the mixture is stirred at 0° C. for 30 minutes. 80 μl of acetyl chloride are then added and the mixture is stirred at ambient temperature for 20 hours. The pyridine is concentrated under reduced pressure, and the residue is taken up in 50 ml of water and is then extracted with 3 times 25 ml of ethyl acetate. The combined organic phases are washed with water, dried over magnesium sulfate and concentrated to dryness under reduced pressure. The residue is purified by flash chromatography on silica gel (60; 30-75 μm), elution being carried out with a mixture of dichloromethane and methanol (97-3 by volume). After recrystallizing from 10 ml of diethyl ether, 135 mg of [4-(3,5-dimethoxyphenyl)piperazin-1-yl](1-acetyl-2-phenyl-1H-pyrrol-3-yl)methanone are thus obtained in the form of white crystals, the characteristics of which are as follows:

Mass spectrum (EI): m/z=433 (M+)

Melting point (Kofler bench)=150° C.

### EXAMPLE 64

[4-(3,5-Dimethoxyphenyl)piperazin-1-yl][1-(3-pyridyl)methyl-2-phenyl-1H-pyrrol-3-yl]methanone

300 mg of [4-(3,5-dimethoxyphenyl)piperazin-1-yl](2-phenyl-1H-pyrrol-3-yl)methanone, obtained in example 21, are dissolved in 15 ml of pyridine. After cooling to 0° C., 61 mg of sodium hydride at 60% in oil, prewashed by settling out in toluene, are added portionwise and the mixture is stirred at 0° C. for 30 minutes. 291 mg of 3-bromomethylpyridine hydrobromide are then added and the mixture is stirred at ambient temperature for 20 hours. A further 61 mg of sodium hydride at 60% in oil, prewashed by settling out in toluene, and 291 mg of 3-bromomethylpyridine are then added and the mixture is heated at 60° C. for 6 hours. The pyridine is concentrated under reduced pressure and the residue is taken up in 50 ml of water and then extracted with 3 times 25 ml of ethyl acetate. The combined organic phases are washed with water, dried over magnesium sulfate and concentrated to dryness under reduced pressure. The residue is purified by flash chromatography on silica gel (60; 30-75 μm), elution being carried out with a mixture of dichloromethane and ethanol (96.5-3.5 by volume). 50 mg of [4-(3,5-dimethoxyphenyl)piperazin-1-yl][1-(3-pyridyl)methyl-2-phenyl-1H-pyrrol-3-yl]methanone are thus obtained in the form of an orange foam, the characteristic of which is as follows:

Mass spectrum (EI): m/z=482 (M+).

### EXAMPLE 65

3-[4-(2-Methoxycarbonylmethyl-4-phenyl-1H-pyrrole-3-carbonyl)piperazin-1-yl]benzamide

374 mg of 3-[4-(4-phenyl-1H-pyrrol-3-ylcarbonyl)piperazin-1-yl]benzamide, prepared in example 58, are dissolved in 10 ml of anhydrous dimethylformamide (DMF). After cooling to 0° C., 44 mg of sodium hydride at 60% in oil, prewashed by settling out in toluene, are added portionwise and the mixture is stirred at 0° C. for 30 minutes. 168 mg of methyl bromoacetate are then added and the mixture is stirred at ambient temperature for 20 hours.

The pyridine is concentrated under reduced pressure and the residue is taken up in 50 ml of water and then extracted with 3 times 25 ml of ethyl acetate. After drying over magnesium sulfate and concentrating under reduced pressure, the residue is purified by flash chromatography silica gel (60; 30-75 μm), elution being carried out with a mixture of dichloromethane and methanol (96.5-3.5 by volume), then crystallization from 10 ml of diethyl ether. 400 mg of 3-[4-(2-methoxycarbonylmethyl-4-phenyl-1H-pyrrol-3-ylcarbonyl)piperazin-1-yl]benzamide are thus obtained in the form of an amorphous beige solid, the characteristic of which is as follows:

Mass spectrum (EI): m/z=446 (M+)

### EXAMPLE 66

3-[4-(1-Hydroxyethyl-4-phenyl-1H-pyrrol-3-ylcarbonyl)piperazin-1-yl]benzamide

Stage 1: 374 mg of 3-[4-(4-phenyl-1H-pyrrol-3-ylcarbonyl)piperazin-1-yl]benzamide, prepared in example 58, are dissolved in 10 ml of anhydrous dimethylformamide (DMF). After cooling to 0° C., 44 mg of sodium hydride at 60% in oil, prewashed by settling out in toluene, are added portionwise and the mixture is stirred at 0° C. for 30 minutes. 263 mg of (2-bromoethoxy)-tert-butyldimethylsilane are then added and the mixture is stirred at ambient temperature for 20 hours. The reaction medium is taken up in 50 ml of water and then extracted with 3 times 25 ml of ethyl acetate. After drying over magnesium sulfate and concentrating under reduced pressure, the residue is purified by flash chromatography on silica gel (60; 30-75 μm), elution being carried out with a mixture of dichloromethane and methanol (96.5-3.5 by volume), then crystallization from 10 ml diethyl ether. 405 mg of 3-{4-[1-(tert-butyldimethylsilanyloxy)ethyl-4-phenyl-1H-pyrrol-3-yl-carbonyl]piperazin-1-yl}benzamide are thus obtained in the form of a beige solid used as is in the following stage.

Stage 2: 6 ml of a 1M tetra-N-butylammonium fluoride solution in tetrahydrofuran are added to a solution of 400 mg of 3-{4-[1-(tert-butyldimethylsilanyloxy)ethyl-4-phenyl-1H-pyrrol-3-ylcarbonyl]piperazin-1-yl}benzamide in 12 ml of tetrahydrofuran. After stirring at 20° C. for 20 hours, 50 ml of ethyl acetate are added and the product is washed with 3 times 25 ml of water, dried over magnesium sulfate and concentrated to dryness under reduced pressure. The residue obtained is purified by flash chromatography on silica gel (60; 30-75 μm), elution being carried out in a mixture of dichloromethane and methanol (95/5 by volume). After recrystallizing from 15 ml of diisopropyl ether, 210 mg of 3-[4-(1-hydroxyethyl-4-phenyl-1H-pyrrol-3-yl-carbonyl)piperazin-1-yl]benzamide are thus obtained in the form of an amorphous beige solid, the characteristic of which is as follows:

Mass spectrum (EI): m/z=418 (M+)

### EXAMPLE 67

[4-(3,5-Dimethoxyphenyl)piperazin-1-yl](2-methylsulfonyl-4-phenyl-1H-imidazol-5-yl)methanone

Stage: 13 g of ethyl 2-mercapto-4-phenyl-1H-imidazol-5-ylcarboxylate, which can be prepared according to Chem. Pharm. Bull. 1984, 32 (7), 2536-43, are dissolved in 500 ml of methanol. After cooling to 0° C., 3.4 g of sodium methoxide are added portionwise and stirring is carried out for 30 minutes, allowing the mixture to return to ambient temperature. The mixture is again cooled to 0° C., a solution of 3.3 g of methyl iodide in 25 ml of methanol is added dropwise, and the mixture is then brought to reflux for 8 hours. The methanol is concentrated under reduced pressure and the residue is taken up with 150 ml of ethyl acetate and 150 ml of water. The organic phase is separated by settling, washed with water, dried over magnesium sulfate and concentrated under reduced pressure. 13 g of ethyl 2-methylsufanyl-4-phenyl-1H-imidazol-5-yl-carboxylate are thus obtained in the form of an orange-colored oil, used as is in the next stage, the characteristic of which is as follows:

Mass spectrum (EI): m/z=262 (M+)

Stage 2: 7 g of ethyl 2-methylsufanyl-4-phenyl-1H-imidazol-5-ylcarboxylate are dissolved in 200 ml of methanol, and then 24.6 g of oxone® or potassium peroxomonosulfate (2 KHSO₅. KHSO₄. K₂SO₄), in solution in 100 ml of water are added at 10-20° C. After stirring at ambient temperature for 20 hours, 200 ml of water are added and the mixture is extracted three times with 100 ml of ethyl acetate. The combined organic phases are washed with water, washed with a saturated aqueous sodium chloride solution, dried over magnesium sulfate and concentrated under reduced pressure. 6 g of ethyl 2-methylsulfonyl-4-phenyl-1H-imidazol-5-ylcarboxylate are thus obtained in the form of a white solid, used as is in the next stage, the characteristic of which is as follows:

Mass spectrum (EI): m/z=294 (M+)

Stage 3: 1.5 g of ethyl 2-methylsufonyl-4-phenyl-1H-imidazol-5-ylcarboxylate are dissolved in 20 ml of methanol, then a solution of 0.37 g of potassium hydroxide in 10 ml of water is added and the mixture is stirred at ambient temperature for 72 h. After concentrating the methanol under reduced pressure, the residue is taken up in 20 ml of water and brought to a pH of 2 by adding a 1N aqueous hydrochloric acid solution. The precipitate formed is filtered off, washed successively with water and with diisopropyl ether and dried in the oven at 50° C. 1.2 g of 2-methylsufonyl-4-phenyl-1H-imidazol-5-ylcarboxylic acid are thus obtained in the form of an off-white solid, used as is in the following stage, the characteristic of which is as follows:

Mass spectrum (EI): m/z=266 (M+)

Stage 4: The reaction is carried out as in example 5 but, on the one hand, from 444 mg of 2-methylsufonyl-4-phenyl-1H-imidazol-5-ylcarboxylic acid and, on the other hand, from 333 mg of (3,5-dimethoxyphenyl)piperazine, in 37.5 ml of dichloromethane, in the presence of 316 mg from 1-(3-dimethylaminopropyl)-3-ethylcarbodiimide hydrochloride (EDCl) and 223 mg of 1-hydroxybenzotriazole hydrate (HOBT), with stirring at ambient temperature for 72 hours. After purifying by flash chromatography on silica gel (60; 30-75 μm), elution being carried out with a mixture cyclohexane and ethyl acetate (50/50 by volume), then recrystallization from 20 ml of diisopropyl ether, 450 mg of [4-(3,5-dimethoxyphenyl)piperazin-1-yl](2-methylsulfonyl-4-phenyl-1H-imidazol-5-yl)methanone are obtained in the form of a beige solid, the characteristic of which is as follows:

Mass spectrum (EI): m/z=470 (M+)

### EXAMPLE 68

[4-(3-Hydroxymethylphenyl)piperazin-1-yl](4-phenyl-1H-imidazol-5-yl)methanone

The reaction is carried out as in example 5 but, on the one hand, from 121 mg of 4-phenyl-1-H-imidazol-4-ylcarboxylic acid, prepared as in stage 1 of example 3, and, on the other hand, from 170 mg of 1-(3-hydroxymethylphenyl)piperazine dihydrochloride, prepared as in stage 1 of example 57, in the presence of 135 mg of 1-(3-dimethylaminopropyl)-3-ethylcarbodiimide hydrochloride (EDCl), 95 mg of 1-hydroxybenzotriazole hydrate (HOBT) and 198 μl of triethylamine, with stirring at ambient temperature for 20 hours. After purifying by flash chromatography on silica gel (60; 30-75 μm), elution being carried out with a gradient of mixtures of dichloromethane and methanol (from 98/2 to 95/5 by volume), 52 mg of [4-(3-hydroxymethyl-phenyl)piperazin-1-yl](4-phenyl-1H-imidazol-5-yl)methanone are obtained in the form of a white foam, the characteristics of which are as follows:

Mass spectrum (EI): m/z=362 (M+)

¹H NMR spectrum (400 MHz)—δ in ppm—d6-DMSO: from 2.83 to 3.91 (very broad m, 8H); 4.44 (s, 2H); 5.06 (very broad m, 1H); 6.78 (m, 2H); 6.90 (broad s, 1H); 7.17 (t, J=7.5 Hz, 1H); 7.31 (broad d, J=8.0 Hz, 1H); 7.42 (broad t, J=8.0 Hz, 2H); 7.62 (broad d, J=8.0 Hz, 1H); 7.82 (s, 1H); 12.75 (very broad m, 1H).

### EXAMPLE 69

[4-(3,5-Hydroxymethylphenyl)piperazin-1-yl](1-methyl-4-phenyl-1H-pyrrol-3-yl)methanone

The reaction is carried out as in example 5 but, on the one hand, from 133 mg of 1-methyl-4-phenyl-1H-pyrrol-3-ylcarboxylic acid, which can be prepared according to Med. Chem. Res. (1997), 7(2), 98-108, and from 175 mg of 1-(3-hydroxymethylphenyl)piperazine dihydrochloride, prepared as in stage 1 of example 57, in 25 ml of dichloromethane, in the presence of 139 mg of 1-(3-dimethylaminopropyl)-3-ethylcarbodiimide hydrochloride (EDCl), 98 mg of 1-hydroxybenzotriazole hydrate (HOBT) and 204 μl of triethylamine, with stirring at ambient temperature for 20 hours. After purifying the base by flash chromatography on silica gel (60; 30-75 μm), elution being carried out with a mixture of dichloromethane and methanol (from 98/2 to 95/5 by volume), then crystallization from diisopropyl ether, 140 mg of [4-(3,5-dimethoxyphenyl)piperazin-1-yl](1-methyl-4-phenyl-1H-pyrrol-3-yl)methanone hydrochloride are obtained in the form of a beige powder, the characteristics of which are as follows:

Mass spectrum (EI): m/z=375 (M⁺)

¹H NMR spectrum (400 MHz)—δ in ppm—in d6-DMSO: from 2.58 to 3.20 (very broad m, 4H); from 3.35 to 3.65 (very broad m, 4H); 3.69 (s, 3H); 4.41 (d, J=5.5 Hz, 2H); 5.06 (t, J=5.5 Hz, 1H); 6.70 (large dd, J=2.0 and 7.5 Hz, 1H); 6.75 (broad d, J=7.5 Hz, 1H); 6.81 (t, J=2.0 Hz, 1H); 7.00 (d, J=2.5 Hz, 1H); 7.05 (d, J=2.5 Hz, 1H); from 7.12 to 7.22 (m, 2H); 7.32 (m, 4H).

### EXAMPLE 70

3-[4-(2-Methylsulfonyl-4-phenyl-1H-imidazol-4-ylcarbonyl)piperazin-1-yl]benzonitrile

The reaction is carried out as in example 5 but, on the one hand, from 600 mg of 2-methylsufonyl-4-phenyl-1H-imidazol-5-ylcarboxylic acid, prepared in stage 3 of example 67, and from 528 mg of 1-(3-cyanophenyl)piperazine dihydrochloride, prepared according to Tetrahedron Lett. 2000, 56(24), 4107-10, in 50 ml of dichloromethane, in the presence of 428 mg of 1-(3-dimethylaminopropyl)-3-ethylcarbodiimide hydrochloride (EDCl), 301 mg of 1-hydroxybenzotriazole hydrate (HOBT) and 627 μl of triethylamine, with stirring at ambient temperature for 20 hours. After purifying the base by flash chromatography on silica gel (60; 30-75 μm), elution being carried out from ethyl acetate, then crystallization from diisopropyl ether, 610 mg of 3-[4-(2-methylsulfonyl-4-phenyl-1H-imidazol-4-ylcarbonyl)piperazin-1-yl]benzonitrile are obtained in the form of white crystals, the characteristics of which are as follows:

Mass spectrum (EI): m/z=435 (M⁺)

Melting point (Kofler bench)=198° C.

### EXAMPLE 71

3-[4-(2-Methylsulfonyl-4-phenyl-1H-imidazol-4-ylcarbonyl)piperazin-1-yl]benzamide

The reaction is carried out as in example 5 but, on the one hand, from 600 mg 2-methylsufonyl-4-phenyl-1H-imidazol-5-ylcarboxylic acid, prepared in stage 3 of example 67, and from 564 mg of 1-(3-carboxamidophenyl)piperazine dihydrochloride in 68 ml of dichloromethane, in the presence of 428 mg of 1-(3-dimethylaminopropyl)-3-ethylcarbodiimide hydrochloride (EDCl), 301 mg of 1-hydroxybenzotriazole hydrate (HOBT) and 627 μl of triethylamine, with stirring at ambient temperature for 20 hours. After purifying the base by flash chromatography on silica gel (60; 30-75 μm), elution being carried out with ethyl acetate, then taking it up in diisopropyl ether, 180 mg of 3-[4-(2-methylsulfonyl-4-phenyl-1H-imidazol-4-yl-carbonyl)piperazin-1-yl]benzamide are obtained in the form of an amorphous beige solid, the characteristic of which is as follows:

Mass spectrum (EI): m/z=453 (M⁺)

### EXAMPLE 72

3-[4-(1-Hydroxymethyl-4-phenyl-1H-pyrrol-3-ylcarbonyl)piperazin-1-yl]benzonitrile

500 mg of 3-[4-(4-phenyl-1H-pyrrol-3-ylcarbonyl)piperazin-1-yl]benzonitrile, obtained in example 61, are dissolved in 10 ml of ethanol, then 7 ml of a 37% aqueous formaldehyde solution and 1.543 ml of a 1N aqueous sodium hydroxide solution are successively added, and the mixture is stirred at ambient temperature for 8 days. The reaction mixture is taken up with 50 ml of ethyl acetate and 50 ml of water. The organic phase is separated by settling, washed with water, dried over sodium sulfate and concentrated under reduced pressure. After purifying the base by flash chromatography on silica gel (60; 30-75 μm), elution being carried out with ethyl acetate, then taking it up in diisopropyl ether, 375 mg of 3-[4-(1-hydroxymethyl-4-phenyl-1H-pyrrol-3-ylcarbonyl)piperazin-1-yl]benzonitrile are obtained in the form of an amorphous white solid, the characteristics of which are as follows:

Mass spectrum (EI): m/z=386 (M⁺)

¹H NMR spectrum (400 MHz)—δ in ppm—in d6-DMSO: from 2.65 to 3.20 (very broad m, 4H); from 3.30 to 3.65 (very broad m, 4H); 5.23 (broad s, 2H); 6.62 (broad s, 1H); 7.10 (d, J=2.5 Hz, 1H); 7.16 (m, 4H); 7.24 (dd, J=1.5 and 2.5 Hz, 1H); 7.31 (m, 4H); 7.35 (dd, J=7.5 and 8.5 Hz, 1H).

### EXAMPLE 73

### {2-[4-(3-Carbamoylphenyl)piperazin-1-ylcarbonyl]-3-phenylpyrrol-1-yl}acetic acid

Stage 1: A 4M hydrochloric acid solution in dioxane (11.6 ml) is added to a solution of 2.6 g of tert-butyl 4-(3-cyanophenyl)piperazin-1-ylcarboxylate (obtained as described in stage 1 of example 39) in dioxane (15 ml) and the reaction mixture is then stirred at 20° C. After reaction for 16 hours, an additional portion of 4M hydrochloric acid in dioxane (11 ml) is introduced and then the mixture is stirred at the same temperature for 20 days. The 3-(piperazin-1-yl)benzonitrile formed (2.2 g) is filtered off, washed with ether (15 ml), and then dried.

Stage 2: A mixture of 300 mg of 3-phenyl-1H-pyrrol-2-ylcarboxylic acid, obtained in stage 1 of example 6, 307 mg of 1-(3-dimethylaminopropyl)-3-ethylcarbodiimide hydrochloride (EDCl), 216 mg of 1-hydroxybenzotriazole (HOBT) and 417.2 mg of 3-(piperazin-1-yl)benzonitrile, obtained in the preceding step, in 30 ml of dichloromethane is placed in a 100 ml three-necked flask placed under argon, and then 0.74 ml of triethylamine is added. The reaction mixture is stirred at 20° C. for 16 hours, and the diluted with 50 ml of dichloromethane and 50 ml of water. After separation by settling, extraction is carried out with 20 ml of dichloromethane. The organic extracts are combined, washed with a saturated ammonium chloride solution (20 ml), dried over magnesium sulfate, filtered and evaporated under reduced pressure. The crude compound obtained is purified by chromatography on silica gel (Bondelut cartridge, ref 15111.1000, 26 mm diameter, 20 g of silica 15-40 microns), elution being carried out at a flow rate of 12 ml/min with a 60/40 volv/vol mixture of cyclohexane and ethyl acetate. The fractions containing the expected compound are combined and evaporated under reduced pressure. 3-[4-(3-Phenyl-1H-pyrrol-2-ylcarbonyl)piperazin-1-yl]benzonitrile (260 mg) is isolated.

Stage 3: Using 130.1 mg of 3-[4-(3-phenyl-1H-pyrrol-2-ylcarbonyl)piperazin-1-yl]benzonitrile obtained previously, in solution in dimethylformamide (1.5 ml); 13 mg of sodium hydride and 61.4 mg of methyl bromoacetate are then added and the mixture is left to react at 20° C. for 1.5 hours. Since the reaction is incomplete, an additional portion of sodium hydride (14 mg) and an additional portion of methyl bromoacetate (43 μl) are introduced. After reaction for one hour, the reaction mixture is treated in the following way: dilution with water (20 ml) and ethyl acetate (20 ml), separation by settling, extraction with ethyl acetate (2×15 ml). The organic extracts are combined, dried over magnesium sulfate, and then evaporated under reduced pressure. The compound obtained is purified by chromatography on silica gel (AIT cartridge, ref FC-25Si-HP), elution being carried out with a 90/10 vol/vol mixture of dichloromethane and methanol at a flow rate of 10 ml/min. The fractions containing the expected compound are combined and evaporated under reduced pressure, providing methyl {2-[4-(3-cyanophenyl)piperazin-1-ylcarbonyl]-3-phenylpyrrol-1-yl}acetate (95.5 mg).

Stage 4: A solution of 0.1M sodium hydroxide (491 μl) and methanol (3 ml) is added to a 50 ml round-bottomed flask containing 95 mg of methyl {2-[4-(3-cyano-phenyl)piperazine-1-ylcarbonyl]-3-phenylpyrrol-1-yl}acetate obtained previously. The reaction mixture is stirred at reflux for 48 hours. Since the reaction is not complete, an additional portion of sodium hydroxide (250 μl) is introduced while maintaining the reflux overnight. The reaction mixture is evaporated to dryness, and then purified by reverse-phase high performance chromatography (injection volume 5 ml DMSO, C18 100-10, 250×40 mm Nucleodur column, ref 762020, series No. 3051181, batch 2023) using a water/acetonitrile gradient (comprising 0.07% of trifluoroacetic acid, from 95/5 to 5/95, proportions by volume, over 52 minutes at a flow rate of 75 ml/min). The fractions containing the expected compound are combined and evaporated. The solid obtained is taken up and triturated in 3 ml of ethyl ether, filtered off and dried, to give {2-[4-(3-carbamoylphenyl)piperazin-1-ylcarbonyl]-3-phenylpyrrol-1-yl}acetic acid (22 mg).

Melting point: 132° C. (Kofler bench)

### EXAMPLE 74

3-[4-(2-Phenyl-1H-pyrrol-3-ylcarbonyl)piperazin-1-yl]benzonitrile

The reaction is carried out as in example 5 but, on the one hand, from 500 mg of 2-phenyl-1H-pyrrol-3-ylcarboxylic acid, prepared in stage 2 of example 21, and from 700 mg of 1-(3-cyanophenyl)piperazine dihydrochloride, prepared according to Tetrahedron Lett. 2000, 56(24), 4107-10, in 50 ml of dichloromethane, in the presence of 560 mg of 1-(3-dimethylaminopropyl)-3-ethylcarbodiimide hydrochloride (EDCl), 36 mg of 1-hydroxybenzotriazole hydrate (HOBT) and 1.1 ml of triethylamine, with stirring at ambient temperature for 72 hours. After purifying the base by flash chromatography on silica gel (60; 30-75 μm), elution being carried out with a mixture of dichloromethane and ethanol (95/5 by volume), then crystallization from diethyl ether, 450 mg of 3-[4-(2-phenyl-1H-pyrrol-3-ylcarbonyl)piperazin-1-yl]benzonitrile are obtained in the form of white crystals, the characteristics of which are as follows:

Mass spectrum (EI): m/z=356 (M⁺)

Melting point (Kofler bench)=80° C.

### EXAMPLE 75

3-[4-(2-Hydroxy-4-phenyl-1H-imidazol-5-ylcarbonyl)piperazin-1-yl]-benzonitrile

The reaction is carried out as in example 5 but, on the one hand, from 137 mg of 2-hydroxy-4-phenyl-1H-imidazol-5-ylcarboxylic acid, prepares according to Heterocycles 1984, 22(8), 1763-69, and from 192 mg of 1-(3-cyanophenyl)piperazine dihydrochloride, prepared according to Tetrahedron Lett. 2000, 56(24), 4107-10, in 25 ml of dichloromethane, in the presence of 141 mg of 1-(3-dimethylaminopropyl)-3-ethylcarbodiimide hydrochloride (EDCl), 9 mg of 1-hydroxybenzotriazole hydrate (HOBT) and 0.28 ml of triethylamine, with stirring at ambient temperature for 72 hours. After purifying the base by flash chromatography on silica gel (60; 30-75 μm), elution being carried out with a mixture of dichloromethane and ethanol (95/5 by volume), then crystallization from diethyl ether, 185 mg of 3-[4-(2-hydroxy-4-phenyl-1H-imidazol-5-ylcarbonyl)piperazin-1-yl]benzonitrile are obtained in the form of white crystals, the characteristics of which are as follows:

Mass spectrum (EI): m/z=373 (M⁺)

Melting point (Kofler bench)=198° C.

¹H NMR spectrum (300 MHz)—δ in ppm—in d6-DMSO: 3.23 (broad m, 4H); 3.61 (broad m, 4H); from 7.17 to 7.46 (m, 9H); 7.75 (broad d, J=8.0 Hz, 2H).

### EXAMPLE 76

[4-(3-Hydroxymethylphenyl)piperazin-1-yl](2-methyl-4-phenyl-1H-imidazol-5-yl)methanone

Stage 1: 3.5 g of ethyl 2-methyl-4-phenyl-1H-imidazol-5-ylcarboxylate, prepared according to Heteroatom. Chemistry 1996, 7(3), 187-94, are dissolved in 60 ml of ethanol, then a solution of 1 g of potassium hydroxide in 30 ml of water is added and the mixture is stirred at reflux for 20 h. After concentrating the methanol under reduced pressure, the residue is taken up in 20 ml of water and brought to a pH of 2 by adding a 1N aqueous hydrochloric acid solution. The precipitate formed is filtered off, washed successively with water and diisopropyl ether, and dried in an oven at 50° C. 3 g of 2-methyl-4-phenyl-1H-imidazol-5-ylcarboxylic acid are thus obtained in the form of a beige solid, used as is in the following stage, the characteristic of which is as follows:

Mass spectrum (EI): m/z=202 (M+)

Stage 2: The reaction is carried out as in example 5 but, on the one hand, from 202 mg of 2-methyl-4-phenyl-1H-imidazol-5-ylcarboxylic acid and, on the other hand, from 265 mg of (3-hydroxyphenyl)piperazine dihydrochloride, obtained as in stage 1 of example 57, in 25 ml of dichloromethane, in the presence of 211 mg of 1-(3-dimethylaminopropyl)-3-ethylcarbodiimide hydrochloride (EDCl), 148 mg of 1-hydroxybenzotriazole hydrate (HOBT) and from 422 μl of triethylamine, with stirring at ambient temperature for 20 hours. After purifying by flash chromatography on silica gel (60; 30-75 μm), elution being carried out with a mixture of dichloromethane and methanol (95/5 by volume), then taking up in 20 ml of diisopropyl ether, 60 mg of [4-(3-hydroxymethylphenyl)piperazin-1-yl](2-methyl-4-phenyl-1H-imidazol-5-yl)methanone are obtained in the form of an amorphous beige solid, the characteristic of which is as follows:

Mass spectrum (EI): m/z=376 (M+)

### EXAMPLE 77

3-[4-(1-Hydroxyethyl-2-phenyl-1H-pyrrol-3-ylcarbonyl)piperazin-1-yl]-benzamide

Stage 1: 450 mg of 3-[4-(2-phenyl-1H-pyrrol-3-ylcarbonyl)piperazin-1-yl]benzamide, prepared in example 25, are dissolved in 20 ml of anhydrous pyridine. After cooling to 0° C., 72 mg of sodium hydride at 60% in oil, prewashed by settling out in toluene, are added portionwise and the mixture is stirred at 0° C. for 30 minutes. 0.28 ml of (2-bromoethoxy)-tert-butyldimethyl-silane is then added and the mixture is stirred at ambient temperature for 20 hours and then at 60° C. for 4 hours. The pyridine is concentrated under reduced pressure and the residue is taken up in 50 ml of water and then extracted with 3 times 25 ml of ethyl acetate. After drying on magnesium sulfate and concentrating under reduced pressure, the residue is purified by flash chromatography on silica gel (60; 30-75 μm), elution being carried out with a mixture of dichloromethane and ethanol (90-10 by volume). 95 mg of 3-{4-[1-(tert-butyldimethylsilanyloxy)ethyl-2-phenyl-1H-pyrrol-3-ylcarbonyl]-piperazin-1-yl}benzamide are thus obtained in the form of an orange oil, used as is in the following stage.

Stage 2: 1.4 ml of a 1M tetra-N-butylammonium fluoride solution in tetrahydrofuran are added to a solution of 95 mg of 3-{4-[1-(tert-butyldimethylsilanyloxy)ethyl-2-phenyl-1H-pyrrol-3-ylcarbonyl]piperazin-1-yl}-benzamide in 3.5 ml of tetrahydrofuran. After stirring for 20 hours at 20° C., 50 ml of ethyl acetate are added and the product is washed with 3 times 25 ml of water, dried over magnesium sulfate and concentrated to dryness under reduced pressure. The residue obtained is purified by flash chromatography on silica gel (60; 30-75 μm), elution being carried out with a mixture of dichloromethane and ethanol (90/10 by volume). 65 mg of 3-[4-(1-hydroxyethyl-2-phenyl-1H-pyrrol-3-ylcarbonyl)piperazin-1-yl]benzamide are thus obtained in the form of a beige foam, the characteristic of which is as follows:

Mass spectrum (EI): m/z=418 (M+)

### EXAMPLE 78

[4-(3-Hydroxymethylphenyl)piperazin-1-yl](2-mercapto-4-phenyl-1H-imidazol-5-yl)methanone

The reaction is carried out as in example 5 but, on the one hand, from 265 mg of 2-mercapto-4-phenyl-1H-imidazol-5-ylcarboxylic acid, obtained according to Chem. Pharm. Bull. 1984, 32(7), 2536-43, and, on the other hand, from 220 mg of (3-hydroxyphenyl)piperazine dihydrochloride, obtained as in stage 1 of exemple 57, in 25 ml of dichloromethane, in the presence of 211 mg of 1-(3-dimethylaminopropyl)-3-ethylcarbodiimide hydrochloride (EDCl), 148 mg of 1-hydroxybenzotriazole hydrate (HOBT) and 309 μl of triethylamine, with stirring at ambient temperature for 20 hours. After purifying by flash chromatography on silica gel (60; 30-75 μm), elution being carried out with a mixture of dichloromethane and methanol (95/5 by volume), then taking up in 20 ml of diisopropyl ether, 175 mg of [4-(3-hydroxymethylphenyl)piperazin-1-yl](2-mercapto-4-phenyl-1H-imidazol-5-yl)methanone are obtained in the form of an amorphous beige solid, the characteristic of which is as follows:

Mass spectrum (EI): m/z=394 (M+)

### EXAMPLE 79

[4-(3-Hydroxymethylphenyl)piperazin-1-yl](2-methylsulfanyl-4-phenyl-1H-imidazol-5-yl)methanone

The reaction is carried out as in example 5 but, on the one hand, from 521 mg of 2-mercapto-4-phenyl-1H-imidazol-5-ylcarboxylic acid, obtained as in stage 1 of example 67, and, on the other hand, from 530 mg of (3-hydroxyphenyl)piperazine dihydrochloride, obtained as in stage 1 of example 57, in 50 ml of dichloromethane, in the presence of 422 mg of 1-(3-dimethylaminopropyl)-3-ethylcarbodiimide hydrochloride (EDCl), 297 mg of 1-hydroxybenzotriazole hydrate (HOBT) and 613 μl of triethylamine, with stirring at ambient temperature for 20 hours. After purifying by flash chromatography on silica gel (60; 30-75 μm), elution being carried out with ethyl acetate, then taking up in 20 ml of diisopropyl ether, 100 mg of 4-(3-hydroxymethylphenyl)piperazin-1-yl](2-methylsulfanyl-4-phenyl-1H-imidazol-5-yl)methanone are obtained in the form of a beige foam, the characteristics of which are as follows:

Mass spectrum (EI): m/z=408 (M+)

¹H NMR spectrum (400 MHz)—δ in ppm—in d6-DMSO at 353K: 2.63 (broad s, 3H); from 3.47 to 3.74 (very broad m, 8H); 4.46 (broad s, 2H); 4.81 (broad m, 1H); 6.78 (broad d, J=7.5 Hz, 2H); 6.90 (broad s, 1H); 7.17 (t, J=7.5 Hz, 1H); 7.30 (broad t, J=7.5 Hz, 1H); 7.41 (t, J=7.5 Hz, 2H); 7.61 (broad d, J=7.5 Hz, 2H); 12.6 (broad m, 1H).

### EXAMPLE 80

[4-(3-Hydroxymethylphenyl)piperazin-1-yl](1-methyl-2-phenyl-1H-pyrrol-3-yl)-methanone

The reaction is carried out as in example 5 but, on the one hand, from 157 mg of 1-methyl-2-phenyl-1H-pyrrol-3-ylcarboxylic acid, which can be prepared according to Med. Chem. Res. (1997), 7(2), 98-108, and, on the other hand, from 227 mg of (3-hydroxyphenyl)piperazine dihydrochloride, obtained as in stage 1 of example 57, in 50 ml of dichloromethane, in the presence of 165 mg of 1-(3-dimethylaminopropyl)-3-ethylcarbodiimide hydrochloride (EDCl), 12 mg of 1-hydroxybenzotriazole hydrate (HOBT) and 240 μl of triethylamine, with stirring at ambient temperature for 20 hours. After purifying by flash chromatography on silica gel (60; 30-75 μm), elution being carried out with a mixture of dichloromethane and methanol (95-5 by volume), 115 mg of [4-(3-hydroxymethylphenyl)piperazin-1-yl](1-methyl-2-phenyl-1H-pyrrol-3-yl)methanone are obtained in the form of a white foam, the characteristic of which is as follows:

Mass spectrum (EI): m/z=375 (M+)

### EXAMPLE 81

3-[4-(1-Hydroxyethyl-2-phenyl-1H-pyrrol-3-ylcarbonyl)piperazin-1-yl]-benzonitrile

Stage 1: 430 mg of 3-[4-(2-phenyl-1H-pyrrol-3-ylcarbonyl)piperazin-1-yl]benzonitrile, prepared in example 74, are dissolved in 20 ml of anhydrous pyridine. After cooling to 0° C., 72 mg of sodium hydride at 60% in oil, prewashed by settling out in toluene, are added portionwise and the mixture is stirred at 0° C. for 30 minutes. 0.28 ml of (2-bromoethoxy)-tert-butyldimethylsilane is then added and the mixture is stirred at ambient temperature for 20 hours. The pyridine is concentrated under reduced pressure and the residue is taken up in 50 ml of water and then extracted with 3 times 25 ml of ethyl acetate. After drying on magnesium sulfate and concentrating under reduced pressure, the residue is purified by flash chromatography on silica gel (60; 30-75 μm), elution being carried out with a mixture of dichloromethane and ethanol (90-10 by volume). 550 mg of 3-{4-[1-(tert-butyldimethylsilanyloxy)ethyl-2-phenyl-1H-pyrrol-3-ylcarbonyl]-piperazin-1-yl}benzonitrile are thus obtained in the form of a yellow oil, used as is in the following stage.

Stage 2: 8 ml of a 1M tetra-N-butylammonium fluoride solution in tetrahydrofuran are added to a solution of 550 mg of 3-{4-[1-(tert-butyldimethylsilanyloxy)ethyl-2-phenyl-1H-pyrrol-3-ylcarbonyl]piperazin-1-yl}-benzonitrile in 35 ml of tetrahydrofuran. After stirring for 20 hours at 20° C., 50 ml of ethyl acetate are added and the product is washed with 3 times 25 ml of water, dried over magnesium sulfate and concentrated to dryness under reduced pressure. The residue obtained is purified by flash chromatography on silica gel (60; 30-75 μm), elution being carried out with a mixture of dichloromethane and ethanol (90/10 by volume). After recrystallizing from 7.5 ml of diethyl ether, 140 mg of 3-[4-(1-hydroxyethyl-2-phenyl-1H-pyrrol-3-ylcarbonyl)piperazin-1-yl]benzonitrile are thus obtained in the form of off-white crystals, the characteristics of which are as follows:

Mass spectrum (EI): m/z=400 (M+)

Melting point (Kofler bench)=158° C.

### EXAMPLE 82

3-[4-(2-Amino-4-phenylthiazol-5-ylcarbonyl)piperazin-1-yl]benzonitrile

The reaction is carried out as in example 5 but, on the one hand, from 200 mg of 2-amino-4-phenyl-thiazol-5-ylcarboxylic acid, prepared according to U.S. Pat. No. 3,282,927, and from 252.6 mg of 1-(3-cyanophenyl)piperazine dihydrochloride, prepared according to Tetrahedron Lett. 2000, 56(24), 4107-10, in 20 ml of dichloromethane, in the presence of 191.5 mg of 1-(3-dimethylaminopropyl)-3-ethylcarbodiimide hydrochloride (EDCl), 135 mg of 1-hydroxybenzotriazole hydrate (HOBT) and 281 μl of triethylamine, with stirring at ambient temperature for 20 hours. After purifying by crystallization from 30 ml of ethanol, 200 mg of 3-[4-(2-amino-4-phenylthiazol-5-ylcarbonyl)piperazin-1-yl]benzonitrile are obtained in the form of pale yellow crystals, the characteristics of which are as follows:

Mass spectrum (EI): m/z=389 (M⁺)

Melting point (Kofler bench)=246° C.

¹H NMR spectrum (400 MHz)—δ in ppm—in d6-DMSO: 2.96 (broad m, 4H); 3.44 (broad m, 4H); 7.18 (m, 2H); 7.25 (t, J=2.0 Hz, 1H); from 7.30 to 7.44 (m, 6H); 7.55 (broad d, J=8.0 Hz, 2H).

### EXAMPLE 83

[4-(3-Hydroxymethylphenyl)piperazin-1-yl](2-trifluoromethyl-4-phenyl-1H-imidazol-5-yl)methanone

The reaction is carried out as in example 5 but, on the one hand, from 256 mg of 2-trifluoromethyl-4-phenyl-1H-imidazol-4-ylcarboxylic acid, prepared as in stage 1 of example 54, and, on the other hand, from 265 mg of 1-(3-hydroxymethylphenyl)piperazine dihydrochloride, prepared as in stage 1 of example 57, in 25 ml of dichloromethane, in the presence of 211 mg of 1-(3-dimethylaminopropyl)-3-ethylcarbodiimide hydrochloride (EDCl), 148 mg of 1-hydroxybenzotriazole hydrate (HOBT) and 309 μl of triethylamine, with stirring at ambient temperature for 20 hours. After purifying by flash chromatography on silica gel (60; 30-75 μm), elution being carried out with a mixture of dichloromethane and ethanol (97.5/2.5 by volume), then taking up in diisopropyl ether, 250 mg of [4-(3-hydroxymethylphenyl)piperazin-1-yl](2-trifluoromethyl-4-phenyl-1H-imidazol-5-yl)methanone are obtained in the form of an amorphous beige solid, the characteristic of which is as follows:

Mass spectrum (EI): m/z=430 (M+)

### EXAMPLE 84

3-[4-(2-Trifluoromethyl-4-phenyl-1H-imidazol-5-ylcarbonyl)piperazin-1-yl]-benzonitrile

The reaction is carried out as in example 5 but, on the one hand, from 256 mg of 2-trifluoromethyl-4-phenyl-1H-imidazol-4-ylcarboxylic acid, prepared as in stage 1 of example 54, and, on the other hand, from 260 mg of 1-(3-cyanomethylphenyl)piperazine dihydrochloride, prepared according to Tetrahedron Lett. 2000, 56(24), 4107-10, in the presence of 211 mg of 1-(3-dimethylaminopropyl)-3-ethylcarbodiimide hydrochloride (EDCl), 149 mg of 1-hydroxybenzotriazole hydrate (HOBT) and 309 μl of triethylamine, with stirring at ambient temperature for 20 hours. After purifying by flash chromatography on silica gel (60; 30-75 μm), elution being carried out with a mixture of dichloromethane and methanol (97.5/2.5 by volume), then taking up in diisopropyl ether, 80 mg of 3-[4-(2-trifluoromethyl-4-phenyl-1H-imidazole-5-ylcarbonyl)piperazin-1-yl]benzonitrile are obtained in the form of an amorphous beige solid, the characteristic of which is as follows:

Mass spectrum (EI): m/z=425 (M+)

### EXAMPLE 85

3-[4-(2-Amino-4-phenyl-thiazol-5-ylcarbonyl)piperazin-1-yl]benzamide

The reaction is carried out as in example 5 but, on the one hand, from 200 mg of 2-amino-4-phenylthiazol-5-ylcarboxylic acid, prepared according to U.S. Pat. No. 3,282,927, and from 258 mg of 1-(3-carboxamidophenyl)piperazine dihydrochloride, in 30 ml of dichloromethane, in the presence of 192 mg of 1-(3-dimethylaminopropyl)-3-ethylcarbodiimide hydrochloride (EDCl), 135 mg of 1-hydroxybenzotriazole hydrate (HOBT) and 281 μl of triethylamine, with stirring at ambient temperature for 20 hours. After purifying by flash chromatography on silica gel (60; 30-75 μm), elution being carried out with a gradient of a mixture of dichloromethane and methanol (from 95/5 to 90/10 by volume), 100 mg of 3-[4-(2-amino-4-phenylthiazol-5-ylcarbonyl)piperazin-1-yl]benzamide are obtained in the form of a white solid, the characteristics of which are as follows:

Mass spectrum (EI): m/z=407 (M⁺)

Melting point (Kofler bench)=236° C.

### EXAMPLE 86

[4-(3-Hydroxymethylphenyl)piperazin-1-yl](2-phenyl-1H-pyrrol-3-yl)methanone

The reaction is carried out as in example 5 but, on the one hand, from 256 mg of 2-phenyl-1H-pyrrol-3-ylcarboxylic acid, prepared as in stage 2 of example 21, and, on the other hand, from 265 mg of 1-(3-hydroxymethylphenyl)piperazine dihydrochloride, prepared as in stage 1 of example 57, in the presence of 211 mg of 1-(3-dimethylaminopropyl)-3-ethylcarbodiimide hydrochloride (EDCl), 149 mg of 1-hydroxybenzotriazole hydrate (HOBT) and 309 μl of triethylamine, with stirring at ambient temperature for 20 hours. After purifying by flash chromatography on silica gel (60; 30-75 μm), elution being carried out with a mixture of dichloromethane and ethanol (95/5 by volume), 250 mg of [4-(3-hydroxymethylphenyl)piperazin-1-yl](2-phenyl-1H-pyrrol-3-yl)methanone are obtained in the form of a beige foam, the characteristic of which is as follows:

Mass spectrum (EI): m/z=361 (M+)

### EXAMPLE 87

3-[4-(1-Hydroxyethyl-4-phenyl-1H-pyrrol-3-ylcarbonyl)piperazin-1-yl]-benzonitrile

Stage 1: 500 mg of 3-[4-(4-phenyl-1H-pyrrol-3-ylcarbonyl)piperazin-1-yl]benzonitrile, prepared as in example 61, are dissolved in 15 ml of anhydrous dimethylformamide (DMF). After cooling to 0° C., 62 mg of sodium hydride at 60% in oil, prewashed by settling out in toluene, are added portionwise and the mixture is stirred at 0° C. for 30 minutes. 370 mg of (2-bromoethoxy)-tert-butyldimethylsilane are then added and the mixture is stirred at ambient temperature for 20 hours. The reaction medium is taken up in 50 ml of water and then extracted with 3 times 25 ml of ethyl acetate. After drying over magnesium sulfate and concentrating under reduced pressure, the residue is purified by flash chromatography on silica gel (60; 30-75 μm), elution being carried out with ethyl acetate. 700 mg of 3-{4-[1-(tert-butyldimethyl-silanyloxy)ethyl-4-phenyl-1H-pyrrol-3-ylcarbonyl]piperazin-1-yl}benzonitrile are thus obtained in the form of a yellow oil, used as is in the following stage.

Stage 2: 10 ml of a 1M tetra-N-butylammonium fluoride solution in tetrahydrofuran are added to a solution of 720 mg of 3-{4-[1-(tert-butyidimethylsilanyloxy)ethyl-4-phenyl-1H-pyrrol-3-ylcarbonyl]piperazin-1-yl}-benzonitrile in 15 ml of tetrahydrofuran. After stirring for 20 hours at 20° C., 50 ml of ethyl acetate are added and the product is washed with 3 times 25 ml of water, dried over magnesium sulfate and concentrated to dryness under reduced pressure. The residue obtained is purified by flash chromatography on silica gel (60; 30-75 μm), elution being carried out with a mixture of dichloromethane and ethanol (92.5/7.5 by volume). After taking up in diethyl ether, 400 mg of 3-[4-(1-hydroxyethyl-4-phenyl-1H-pyrrol-3-ylcarbonyl)piperazin-1-yl]benzonitrile are thus obtained in the form of a beige solid, the characteristic of which is as follows:

Mass spectrum (EI): m/z=400 (M+)

### EXAMPLE 88

[4-(3-Hydroxymethylphenyl)piperazin-1-yl](2-amino-4-phenylthiazol-5-yl)-methanone

The reaction is carried out as in example 5 but, on the one hand, from 324 mg of 2-amino-4-phenylthiazol-5-ylcarboxylic acid, prepared according to U.S. Pat. No. 3,282,927, and from 390 mg of 1-(3-hydroxymethylphenyl)piperazine dihydrochloride, in 30 ml of dichloromethane, in the presence of 310 mg of 1-(3-dimethylaminopropyl)-3-ethylcarbodiimide hydrochloride (EDCl), 219 mg of 1-hydroxybenzotriazole hydrate (HOBT) and 455 μl of triethylamine, with stirring at ambient temperature for 72 hours. After purifying by flash chromatography on silica gel (60; 30-75 μm), elution being carried out with a mixture of dichloromethane and methanol (95/5 by volume), then recrystallization from 10 ml of a mixture of water and isopropanol (80/20 by volume), 130 mg from [4-(3-hydroxymethylphenyl)piperazin-1-yl](2-amino-4-phenylthiazol-5-yl)methanone are obtained in the form of yellow crystals, the characteristics of which are as follows:

Mass spectrum (EI): m/z=394 (M⁺)

Melting point (Kofler bench)=176° C.

### EXAMPLE 89

[4-(3-Hydroxymethylphenyl)piperazin-1-yl](1-hydroxyethyl-2-phenyl-1H-pyrrol-5-yl)methanone

Stage 1: 240 mg of [4-(3-hydroxymethylphenyl)piperazin-1-yl](2-phenyl-1H-pyrrol-3-yl)methanone, prepared as in example 86, are dissolved in 15 ml of anhydrous pyridine. After cooling to 0° C., 40 mg of sodium hydride at 60% in oil, prewashed by settling out in toluene, are added portionwise and the mixture is stirred at 0° C. for 30 minutes. 160 μl of (2-bromoethoxy)-tert-butyldimethylsilane are then added and the mixture is stirred at ambient temperature for 20 hours. The pyridine is concentrated under reduced pressure and the residue is taken up in 25 ml of water and then extracted with 3 times 15 ml of ethyl acetate. After drying over magnesium sulfate and concentrating under reduced pressure, the residue is purified by flash chromatography on silica gel (60; 30-75 μm), elution being carried out with a mixture of dichloromethane and methanol (95/5 by volume). 100 mg of [4-(3-hydroxymethylphenyl)piperazin-1-yl](1-(tert-butyldimethylsilanyloxy)ethyl-2-phenyl-1H-pyrrol-5-yl)methanone are thus obtained in the form of an orange oil, used as is in the following stage.

Stage 2: 1.5 ml of a 1M tetra-N-butylammonium fluoride solution in tetrahydrofuran are added to a solution of 98 mg of [4-(3-hydroxymethylphenyl)piperazin-1-yl](1-(tert-butyldimethylsilanyloxy)ethyl-2-phenyl-1H-pyrrol-5-yl)methanone in 5 ml of tetrahydrofuran. After stirring for 20 hours at 20° C., 50 ml of ethyl acetate are added and the product is washed with 3 times 25 ml of water, dried over magnesium sulfate and concentrated to dryness under reduced pressure. The residue obtained is purified by flash chromatography on silica gel (60; 30-75 μm), elution being carried out with a mixture of dichloromethane and ethanol (95/5 by volume). 70 mg of [4-(3-hydroxymethylphenyl)piperazin-1-yl](1-hydroxyethyl-2-phenyl-1H-pyrrol-5-yl)-methanone are thus obtained in the form of a white foam, the characteristic of which is as follows:

Mass spectrum (EI): m/z=405 (M+)

### EXAMPLE 90

[4-(3,5-Dimethylphenyl)piperazin-1-yl][2-(2-methoxyethyl)amino-4-phenylthiazol-5-yl]methanone

Stage 1: 2.3 g of ethyl 2-(2-methoxyethyl)amino-4-phenylthiazol-5-ylcarboxylate, prepared according to Pharmazie 1987, 42(6), 373-375, are dissolved in 30 ml of ethanol, then 7.8 ml of a 1N aqueous sodium hydroxide solution are added and the mixture is refluxed for 15 minutes. After cooling to ambient temperature, a further 3.5 ml of 1N aqueous sodium hydroxide solution are added and the mixture is brought to reflux for 30 minutes. After concentrating the ethanol under reduced pressure, 20 ml of water are added and extraction is carried out with 20 ml of dichloromethane. The aqueous phase is acidified to a pH of 2 by adding a 1N aqueous hydrochloric acid solution. The precipitate formed is filtered off, and washed with water and with a mixture of methanol and dichloromethane (80/20 by volume). 0.6 g of 2-(2-methoxyethyl)amino-4-phenylthiazol-5-ylcarboxylic acid is thus obtained in the form of a white solid, used as is in the following stage, the characteristic of which is as follows:

Mass spectrum (EI): m/z=278 (M+)

Stage 2: The reaction is carried out as in example 5 but, on the one hand, from 200 mg of 2-(2-methoxyethyl)amino-4-phenylthiazol-5-ylcarboxylic acid and, on the other hand, from 160 mg of (3,5-dimethylphenyl)piperazine, in 20 ml of dichloromethane, in the presence of 151 mg of 1-(3-dimethylaminopropyl)-3-ethylcarbodiimide hydrochloride (EDCl) and 107 mg of 1-hydroxybenzotriazole hydrate (HOBT), with stirring at ambient temperature for 72 hours. After purifying by flash chromatography on silica gel (25 g cartridge, 40-60 μm), elution being carried out with a mixture of cyclohexane and ethyl acetate (50/50 by volume), 143 mg of [4-(3,5-dimethylphenyl)piperazin-1-yl][2-(2-methoxyethyl)amino-4-phenylthiazol-5-yl]-methanone are obtained in the form of a yellow foam, the characteristic of which is as follows:

Mass spectrum (EI): m/z=450 (M+)

### EXAMPLE 91

[4-(3,5-Dimethoxyphenyl)piperazin-1-yl][2-(2-methoxyethyl)amino-4-phenylthiazol-5-yl]methanone

The reaction is carried out as in example 5 but, on the one hand, from 200 mg of 2-(2-methoxyethyl)amino-4-phenylthiazol-5-ylcarboxylic acid, obtained in stage 1 of example 90, and, on the other hand, from 160 mg of (3,5-dimethoxyphenyl)piperazine, in 20 ml of dichloromethane, in the presence of 151 mg of 1-(3-dimethylaminopropyl)-3-ethylcarbodiimide hydrochloride (EDCl) and 107 mg of 1-hydroxybenzotriazole hydrate (HOBT), with stirring at ambient temperature for 20 hours. After purifying by flash chromatography on silica gel (25 g cartridge, 40-60 μm), eluting with a mixture of cyclohexane and ethyl acetate (50/50 by volume), 190 mg of [4-(3,5-dimethoxyphenyl)piperazin-1-yl][2-(2-methoxyethyl)amino-4-phenylthiazol-5-yl]methanone are obtained in the form of a white foam, the characteristics of which are as follows:

Mass spectrum (EI): m/z=482 (M+)

¹H NMR spectrum (400 MHz)—δ in ppm—in d6-DMSO: 2.83 (broad m, 4H); 3.30 (s, 1H); 3.41 (broad m, 4H); 3.49 (q, J=5.5 Hz, 2H); 3.54 (t, J=5.5 Hz, 2H); 3.68 (s, 6H); 5.97 (broad s, 3H); 7.35 (tt, J=1.5 and 7.5 Hz, 1H); 7.42 (broad t, J=7.5 Hz, 2H); 7.56 (broad d, J=7.5 Hz, 2H); 8.04 (broad t, J=5.5 Hz, 1H).

### EXAMPLE 92

3-{4-[1-(Pyridin-3-yl)methyl-4-phenyl-1H-pyrrol-3-ylcarbonyl]piperazin-1-yl}-benzonitrile

300 mg of 3-[4-(4-phenyl-1H-pyrrol-3-ylcarbonyl)piperazin-1-yl]benzonitrile, obtained in example 61, are dissolved in 10 ml of pyridine. After cooling to 0° C., 40 mg of sodium hydride at 60% in oil, prewashed by settling out in toluene, are added portionwise and the mixture is stirred 0° C. for 30 minutes. 253 mg of 3-bromomethylpyridine hydrobromide are then added and the mixture is stirred at ambient temperature for 20 hours. A further 61 mg of sodium hydride at 60% in oil, prewashed by settling out in toluene, and 291 mg of 3-bromomethylpyridine are then added and the mixture is then stirred at ambient temperature for 20 hours. The pyridine is concentrated under reduced pressure and the residue is taken up in 50 ml of water and then extracted with 3 times 25 ml of ethyl acetate. The combined organic phases are washed with water, dried over magnesium sulfate and concentrated to dryness under reduced pressure. The residue is purified by flash chromatography on silica gel (60; 30-75 μm), elution being carried out with a mixture of dichloromethane and methanol (99-1 by volume). After taking up in diisopropyl ether, 180 mg of 3-{4-[1-(pyridin-3-yl)methyl-4-phenyl-1H-pyrrol-3-ylcarbonyl]piperazin-1-yl}benzonitrile are thus obtained in the form of an amorphous beige solid, the characteristics of which are as follows:

Mass spectrum (EI): m/z=447 (M+)

¹H NMR spectrum (300 MHz)—δ in ppm—in d6-DMSO: from 2.63 to 3.20 (very broad m, 4H); from 3.30 to 3.63 (very broad m, 4H); 5.22 (broad s, 2H); 7.17 (m, 4H); 7.20 (d, J=2.5 Hz, 1H); 7.24 (d, J=2.5 Hz, 1H); from 7.30 to 7.45 (m, 6H); 7.76 (t, J=2.0 and 8.0 Hz, 1H); 8.55 (dd, J=2.0 and 5.0 Hz, 1H); 8.61 (dd, J=1.0 and 2.0 Hz, 1H).

### EXAMPLE 93

[4-(3,5-Dimethoxyphenyl)piperazin-1-yl](2-methyl-4-phenylthiazol-5-yl)-methanone

The reaction is carried out as in example 5 but, on the one hand, from 219 mg of 2-methy-4-phenylthiazol-5-ylcarboxylic acid, which can be obtained according to Tetrahedron 2002, 58(42), 8581-89, and, on the other hand, from 223 mg of (3,5-dimethoxyphenyl)piperazine, in 25 ml of dichloromethane, in the presence of 211 mg of 1-(3-dimethylaminopropyl)-3-ethylcarbodiimide hydrochloride (EDCl) and 148 mg of 1-hydroxybenzotriazole hydrate (HOBT), with stirring at ambient temperature for 20 hours. After purifying by flash chromatography on silica gel (25 g cartridge, 40-60 μm), elution being carried out with a mixture of dichloromethane and methanol (97.5/2.5 by volume), 420 mg of [4-(3,5-dimethoxyphenyl)piperazin-1-yl](2-methyl-4-phenylthiazol-5-yl)methanone are obtained in the form of a white foam, the characteristic of which is as follows:

Mass spectrum (EI): m/z=423 (M+)

### EXAMPLE 94

3-[4-(2-Methyl-4-phenylthiazol-5-ylcarbonyl)piperazin-1-yl]benzamide

The reaction is carried out as in example 5 but, on the one hand, from 219 mg of 2-methyl-4-phenylthiazol-5-ylcarboxylic acid, which can be obtained according to Tetrahedron 2002, 58(42), 8581-89, and, on the other hand, from 278 mg of (3-carboxamidophenyl)piperazine dihydrochloride, in 25 ml of dichloromethane, in the presence of 211 mg of 1-(3-dimethylaminopropyl)-3-ethylcarbodiimide hydrochloride (EDCl), 148 mg of 1-hydroxybenzotriazole hydrate (HOBT) and 309 μl of triethylamine, with stirring at ambient temperature for 20 hours. After purifying by flash chromatography on silica gel (60; 30-75 μm), elution being carried out with a mixture of dichloromethane and methanol (97.5/2.5 by volume), then taking up in diisopropyl ether, 340 mg of 3-[4-(2-Methyl-4-phenyl-thiazole-5-carbonyl)-piperazin-1-yl]-benzamide are obtained in the form of an amorphous beige solid, the characteristics of which are as follows:

Mass spectrum (EI): m/z=406 (M+)

¹H NMR spectrum (300 MHz)—δ in ppm—in d6-DMSO: 2.70 (broad m, 2H); 2.76 (s, 3H); 3.21 (broad m, 4H); 3.76 (broad m, 2H); 6.99 (ddd, J=1.5-2.5 and 7.5 Hz, 1H); from 7.23 to 7.34 (m, 4H); 7.39 (tt, J=2.0 and 7.5 Hz, 1H); 7.45 (broad t, J=7.5 Hz, 2H); 7.67 (broad d, J=7.5 Hz, 2H); 7.87 (broad m, 1H).

### EXAMPLE 95

[4-(3,5-Dimethoxyphenyl)piperazin-1-yl](2-hydroxy-4-phenylthiazol-5-yl)-methanone

Stage 1: 750 mg of ethyl 2-hydroxy-4-phenylthiazol-5-ylcarboxylate, which can be obtained according to Acta Poloniae Pharmaceutica 1984, 41(6), 633-40, are dissolved in 8 ml of ethanol, then 7.5 ml of a 2.5 N aqueous sodium hydroxide solution are added and the mixture is refluxed for 15 minutes. After concentrating the ethanol under reduced pressure, 20 ml of water are added and the mixture is acidified to a pH of 1 by adding a 1N aqueous hydrochloric acid solution. The precipitate formed is filtered off, and washed with water and diisopropyl ether. 0.6 g of 2-hydroxy-4-phenylthiazol-5-ylcarboxylic acid is thus obtained in the form of a yellow solid, used as is in the following stage, the characteristic of which is as follows:

Mass spectrum (EI): m/z=221 (M+)

Stage 2: The reaction is carried out as in example 5 but, on the one hand, from 400 mg of 2-hydroxy-4-phenylthiazol-5-ylcarboxylic acid, and, on the other hand, from 402 mg of (3,5-dimethoxyphenyl)piperazine, in 50 ml of dichloromethane, in the presence of 381 mg of 1-(3-dimethylaminopropyl)-3-ethylcarbodiimide hydrochloride (EDCl) and 269 mg of 1-hydroxybenzotriazole hydrate (HOBT), with stirring at ambient temperature for 20 hours. After purifying by flash chromatography on silica gel (60; 30-75 μm), elution being carried out with a mixture of dichloromethane and methanol (95/5 by volume), 730 mg of [4-(3,5-dimethoxyphenyl)piperazin-1-yl](2-methyl-4-phenylthiazol-5-yl)methanone are obtained in the form of a viscous yellow oil, the characteristic of which is as follows:

Mass spectrum (EI): m/z=425 (M+)

### EXAMPLE 96

3-[4-{2-Hydroxy-4-phenylthiazol-5-ylcarbonyl)piperazin-1-yl]benzamide

The reaction is carried out as in example 5 but, on the one hand, from 200 mg of 2-hydroxy-4-phenylthiazol-5-ylcarboxylic acid, obtained as in stage 1 of example 95, and, on the other hand, from 251 mg of (3-carboxamidophenyl)piperazine dihydrochloride, in 25 ml of dichloromethane, in the presence of 190 mg of 1-(3-dimethylaminopropyl)-3-ethylcarbodiimide hydrochloride (EDCl), 134 mg of 1-hydroxybenzotriazole hydrate (HOBT) and 279 μl of triethylamine, with stirring at ambient temperature for 20 hours. After purifying by flash chromatography on silica gel (60; 30-75 μm), elution being carried out with ethyl acetate, then taking up in diisopropyl ether, 25 mg of 3-[4-(2-hydroxy-4-phenylthiazol-5-ylcarbonyl)piperazin-1-yl]benzamide are obtained in the form of an amorphous yellow solid, the characteristic of which is as follows:

Mass spectrum (EI): m/z=406 (M+)

### EXAMPLE 97

[4-(3,5-Dimethoxyphenyl)piperazin-1-yl](2-methoxy-4-phenylthiazol-5-yl)-methanone

500 mg of [4-(3,5-dimethoxyphenyl)piperazin-1-yl](2-hydroxy-4-phenylthiazol-5-yl)methanone, obtained as in example 95, are dissolved in 10 ml of methanol and cooled to 0° C. 77 mg of sodium methoxide are then added and the mixture is stirred for 30 minutes, and then 80.5 μl of iodomethane are added and the mixture is stirred at 45° C. for 2 hours and then at ambient temperature for 20 hours. After concentrating the methanol under reduced pressure, the residue is taken up with 50 ml of ethyl acetate and 50 ml of water. The organic phase, which has been separated by settling, is washed with water, dried over magnesium sulfate and concentrated to dryness under reduced pressure. After purifying by flash chromatography on silica gel (60; 30-75 μm), elution being carried out with a mixture of dichloromethane and methanol (97.5/2.5 by volume, then taking up in diisopropyl ether, 495 mg of [4-(3,5-dimethoxyphenyl)piperazin-1-yl](2-methoxy-4-phenylthiazol-5-yl)-methanone are obtained in the form of an amorphous yellow solid, the characteristics of which are as follows:

Mass spectrum (EI): m/z=439 (M+)

¹H NMR spectrum (400 MHz)—δ in ppm—in d6-DMSO: 2.64 (broad m, 4H); 3.13 (s, 3H); 3.34 (broad m, 4H); 3.69 (s, 6H); 5.94 (d, J=2.5 Hz, 2H); 5.99 (t, J=2.5 Hz, 1H); from 7.46 to 7.57 (m, 5H)

### EXAMPLE 98

[4-(3,5-Dimethoxyphenyl)piperazin-1-yl](4-phenylthiazol-5-yl)methanone

The reaction is carried out as in example 5 but, on the one hand, from 205 mg of 4-phenylthiazol-5-ylcarboxylic acid, which can be obtained according to Acta Poloniae Pharmaceutica 1984, 41(6), 633-40, and, on the other hand, from 223 mg of (3,5-dimethoxyphenyl)piperazine, in 25 ml of dichloromethane, in the presence of 211 mg of 1-(3-dimethylaminopropyl)-3-ethylcarbodiimide hydrochloride (EDCl) and 148 mg of 1-hydroxybenzotriazole hydrate (HOBT), with stirring at ambient temperature for 20 hours. After purifying by flash chromatography on silica gel (60; 30-75 μm), elution being carried out with a mixture of dichloromethane and methanol (97.5/2.5 by volume), then taking up in diisopropyl ether, 390 mg of [4-(3,5-dimethoxyphenyl)piperazin-1-yl](4-phenylthiazol-5-yl)methanone are obtained in the form of an amorphous yellow solid, the characteristic of which is as follows:

Mass spectrum (EI): m/z=405 (M+)

### EXAMPLE 99

3-[4-(2-Hydroxy-4-phenylthiazol-5-ylcarbonyl)piperazin-1-yl]benzamide

The reaction is carried out as in example 5 but, on the one hand, from 308 mg of 4-phenylthiazol-5-ylcarboxylic acid, which can be obtained according to Acta Poloniae Pharmaceutica 1984, 41(6), 633-40, and, on the other hand, from 417 mg of (3-carboxamidophenyl)piperazine dihydrochloride, in 37.5 ml of dichloromethane, in the presence of 316 mg of 1-(3-dimethylaminopropyl)-3-ethylcarbodiimide hydrochloride (EDCl), 223 mg of 1-hydroxybenzotriazole hydrate (HOBT) and 464 μl of triethylamine, with stirring at ambient temperature for 20 hours. After purifying by flash chromatography on silica gel (60; 30-75 μm), elution being carried out with a mixture of dichloromethane and methanol (95/5 by volume), then taking up in diisopropyl ether, 500 mg of 3-[4-(2-hydroxy-4-phenylthiazol-5-ylcarbonyl)piperazin-1-yl]benzamide are obtained in the form of an amorphous beige solid, the characteristics of which are as follows:

Mass spectrum (EI): m/z=392 (M+)

¹H NMR spectrum (300 MHz)—δ in ppm—in d6-DMSO: 2.70 (broad m, 2H); 3.20 (broad m, 4H); 3.79 (broad m, 2H); 6.98 (ddd, J=1.5-2.5 and 8.0 Hz, 1H); from 7.22 to 7.34 (m, 4H); 7.41 (tt, J=2.0 and 7.5 Hz, 1H); 7.50 (broad t, J=7.5 Hz, 2H); 7.71 (broad d, J=7.5 Hz, 2H); 7.86 (broad m, 1H) 9.31 (s, 1H).

### EXAMPLE 100

3-{4-[2-(2-Methoxyethyl)amino-4-phenylthiazol-5-ylcarbonyl]piperazin-1-yl}-benzonitrile The reaction is carried out as in example 5 but, on the one hand, from 100 mg of 2-(2-methoxyethyl)amino-4-phenylthiazol-5-ylcarboxylic acid, obtained in stage 1 of example 90, and, on the other hand, from 93.5 mg of (3-cyanophenyl)piperazine dihydrochloride, which can be prepared according to Tetrahedron Lett. 2000, 56(24), 4107-10, in 13 ml of dichloromethane and 0.4 ml of DMF, in the presence of 75.5 mg of 1-(3-dimethylaminopropyl)-3-ethylcarbodiimide hydrochloride (EDCl), 53.5 mg of 1-hydroxybenzotriazole hydrate (HOBT) and 103 μl of triethylamine, with stirring at ambient temperature for 20 hours. After purifying by flash chromatography on silica gel (10 g cartridge, 40-60 μm), elution being carried out with a mixture of dichloromethane and methanol (95/5 by volume), 90 mg of [3-{4-[2-(2-methoxyethyl)amino-4-phenylthiazol-5-ylcarbonyl]piperazin-1-yl}benzonitrile are obtained in the form of a white foam, the characteristic of which is as follows:

Mass spectrum (EI): m/z=465 (M+)

### EXAMPLE 101

3-{4-[2-(2-Methoxyethyl)amino-4-phenylthiazol-5-ylcarbonyl]piperazin-1-yl}-benzamide

The reaction is carried out as in example 5 but, on the one hand, from 93 mg of 2-(2-methoxyethyl)amino-4-phenylthiazol-5-ylcarboxylic acid, obtained in stage 1 of example 90, and, on the other hand, from 93 mg of (3-carboxamido-phenyl)piperazine dihydrochloride, in 17 ml of dichloromethane and 0.4 ml of DMF, in the presence of 70.5 mg of 1-(3-dimethylaminopropyl)-3-ethylcarbodiimide hydrochloride (EDCl), 50 mg of 1-hydroxybenzotriazole hydrate (HOBT) and 103 μl of triethylamine, with stirring at ambient temperature for 20 hours. After purifying by flash chromatography on silica gel (10 g cartridge, 40-60 μm), elution being carried out with a mixture of dichloromethane and methanol (95/5 by volume), 60 mg of [3-{4-[2-(2-methoxyethyl)amino-4-phenylthiazol-5-ylcarbonyl]piperazin-1-yl}benzamide are obtained in the form of a white foam, the characteristics of which are as follows:

Mass spectrum (EI): m/z=465 (M+)

¹H NMR spectrum (400 MHz)—δ in ppm—in d6-DMSO: 2.92 (broad m, 4H); 3.30 (masked, 3H); 3.45 (broad m, 4H); 3.48 (q, J=5.5 Hz, 2H); 3.55 (t, J=5.5 Hz, 2H); 6.99 (ddd, J=1.0-2.5 and 8.0 Hz, 1H); from 7.22 to 7.35 (m, 5H); 7.42 (broad t, J=8.0 Hz, 2H); 7.59 (broad d, J=8.0 Hz, 2H); 7.86 (broad m, 1H); 8.03 (broad t, J=5.5 Hz, 1H).

Assessment of the Inhibition of Tubulin Polymerization

Tubulin is purified from pig brains according to published methods (Shelanski et al., 1973, Proc. Natl. Acad. Sci. USA, 70, 765-768. Weingarten et al., 1975, Proc. Natl. Acad. Sci. USA, 72, 1858-1862). Briefly, the brains are ground and centrifuged in an extraction buffer. The tubulin, present in the extract supernatant is subjected to two successive cycles of polymerization at 37° C. and depolymerization at 4° C., before being separated from the MAPs (Microtubule Associated Proteins) by chromatography on a phosphocellulose P11 column (Whatman). The tubulin thus isolated is more than 95% pure. It is stored in a buffer known as RB/2 30% glycerol, the composition of which is 50 mM MES-NaOH [2-(N-morpholino)ethanesulfonic acid], pH 6.8; 0.25 mM MgCl₂; 0.5 mM EGTA; 30% (v/v) glycerol, 0.2 mM GTP (guanosine 5′-triphosphate).

The polymerization of the tubulin into microtubules is monitored by turbidimetry as follows: the tubulin is adjusted to a concentration of 10 μm (1 mg/ml) in the RB/2 30% glycerol buffer, to which are added 1 mM GTP and 6 mM MgCl₂. The polymerization is initiated by increasing the temperature from 6° C. to 37° C. in a cuvette with a 1 cm optical pathlength, placed in a UVIKON 931 spectrophotometer (Kontron) equipped with a thermostatically-regulated cuvette holder. The increase in turbidity of the solution is monitored at 350 nm.

The products are dissolved at 10 mM in DMSO and added at variable concentrations (0.5 to 10 μm) to the tubulin solution before polymerization. The IC₅₀ is defined as the concentration of the product which inhibits the rate of polymerization by 50%. A product with an IC₅₀ of less than or equal to 25 μm is regarded as very active.

A product in accordance with the invention may be of use in inhibiting the proliferation of tumor cells in vitro.

Test for Determining the Inhibition of Proliferation of the HCT116 Human Colon Tumor Line

The proliferation of HCT116 cells is evaluated by mesuring the incorporation of [¹⁴C]-thymidine in the following way. The HCT166 cells (from the ATCC) are cultured in a DMEM medium (Gibco) which contains 10% of fetal calf serum and antibiotics (1% penicillin, 1% streptomycin). To perform the proliferation test, the cells are seeded into 96-well cytostar microplates (Amersham) at the rate of 5000 cells per well. The [¹⁴C]-thymidine (0.1 μCi/well) and the products to be evaluated are subsequently added. Variable concentrations of products up to 10 μM are used; the DMSO (solvent used to dissolve the products) should not exceed 0.5% in the medium. 48 hours after incubation at 37° C., the radioactivity incorporated into the cells is measured by counting the plate in a Tri-Lux counter (Wallac). The IC₅₀ is defined as the concentration of product which reduces the radioactivity by 50% compared with an untreated control. A product with an IC₅₀ of less than 10 μm is regarded as cytotoxic.

Biological Results

| | | | Inhibition of |
|---|---|---|---|
| | | Inhibition of tubulin | HCT116 |
| Example | | polymerization | proliferation |
| No. | Structure | IC 50 (μM) | IC 50 (μM) |
| 1 | 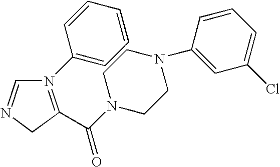 | 2 | 0.0296 |
| 2 | 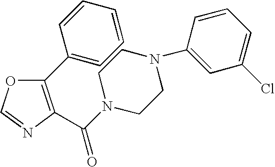 | 22 | 8.7470 |
| 3 | 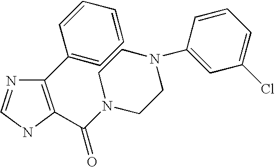 | 1 | 0.0068 |
| 4 | 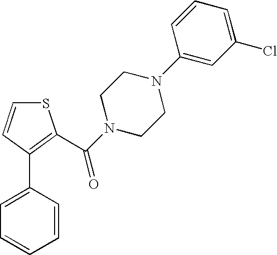 | 2 | 0.0525 |
| 5 | 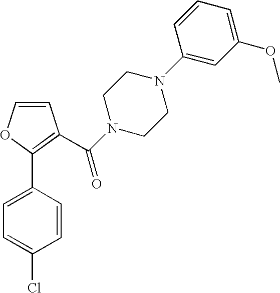 | 11.5 | n.d. |
| 6 | 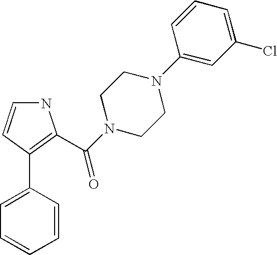 | 0.69 | 0.034 |
| 7 | 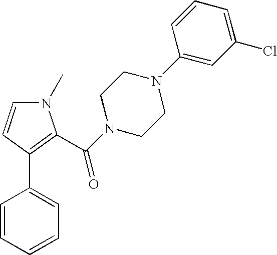 | 12.81 | n.d. |
| 8 | 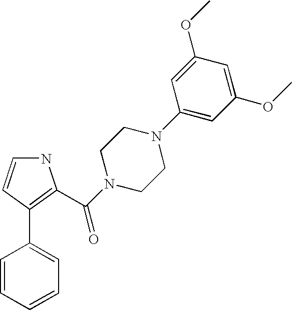 | 1.2 | 0.0029 |
| 9 | 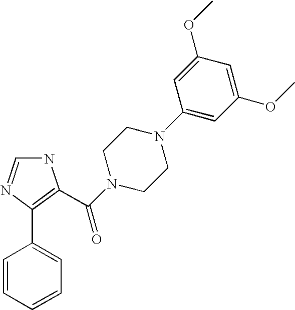 | 0.6 | 0.0013 |
| 10 | 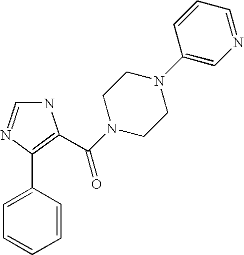 | 3.1 | 0.0885 |
| 11 | 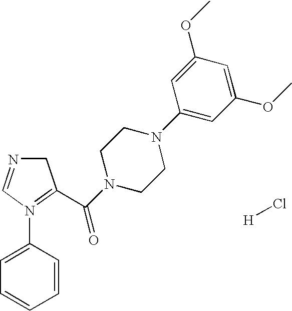 | 2.4 | 0.0457 |
| 12 | 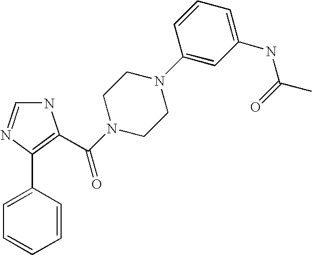 | 3.5 | 1.191 |
| 13 | 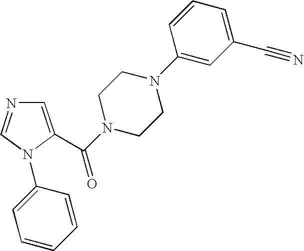 | 2.9 | 0.0909 |
| 14 | 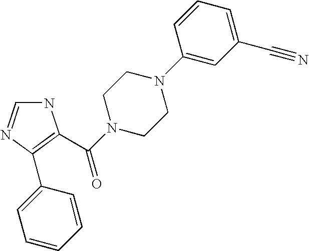 | 1.2 | 0.0074 |
| 15 | 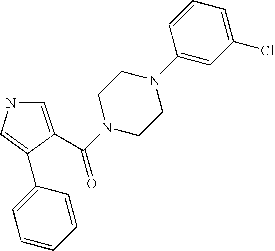 | 2.9 | 0.731 |
| 16 | 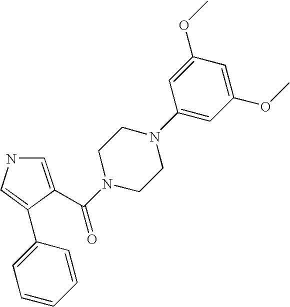 | 2.4 | 0.0085 |
| 17 | 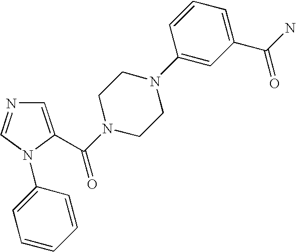 | 5.5 | 0.536 |
| 18 | 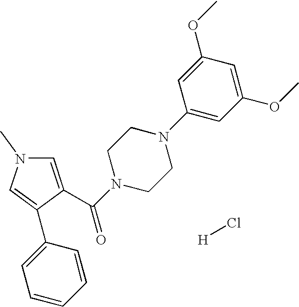 | 0.97 | 0.0038 |
| 19 | 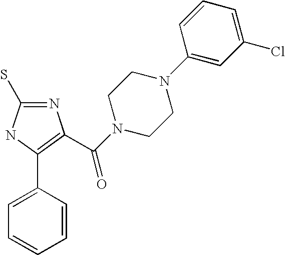 | 0.75 | 0.0287 |
| 20 | 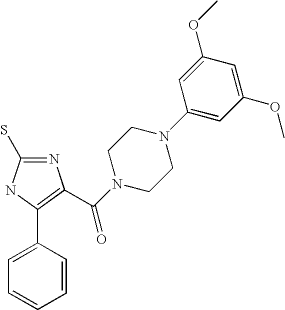 | 0.7 | 0.0117 |
| 21 | 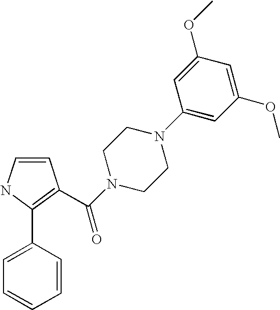 | 9.7 | n.d. |
| 22 | 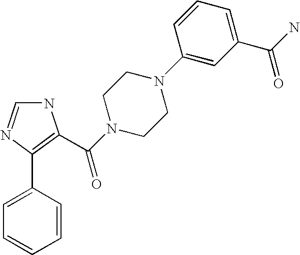 | 1.1 | 0.1927 |
| 23 | 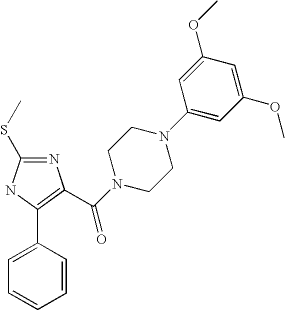 | 0.6 | 0.0017 |
| 24 | 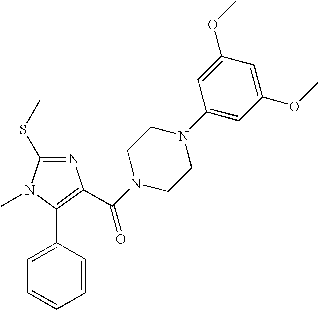 | 9.5 | 0.1143 |
| 25 | 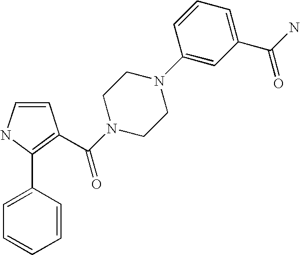 | 9.6 | 0.297 |
| 26 | 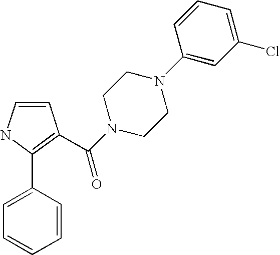 | 3.2 | 0.0752 |
| 27 | 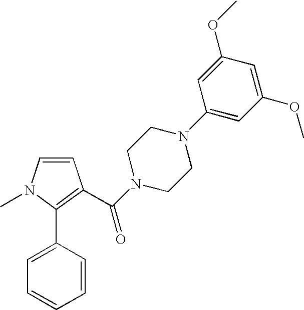 | 1.9 | 0.0151 |
| 28 | 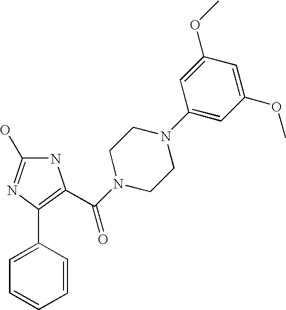 | 1.1 | 0.0015 |
| 29 | 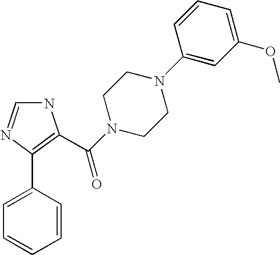 | 0.4 | 0.003 |
| 30 | 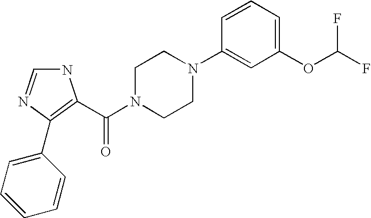 | 0.8 | 0.0064 |
| 31 | 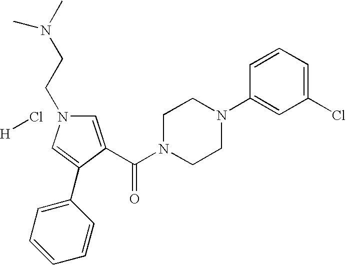 | 25 | n.d. |
| 32 | 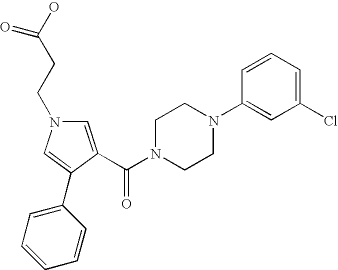 | 2.74 | n.d. |
| 33 | 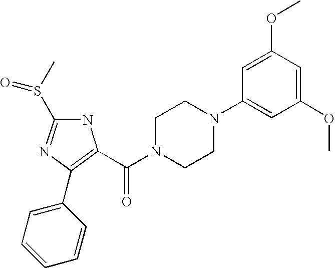 | 1.95 | 0.180 |
| 34 | 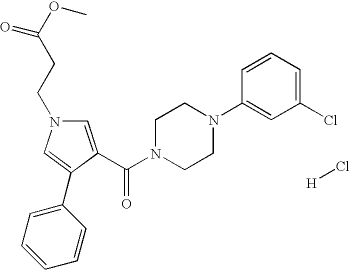 | 2.16 | 0.749 |
| 35 | 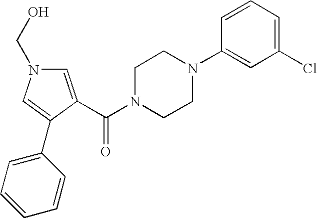 | 0.34 | 0.0069 |
| 36 | 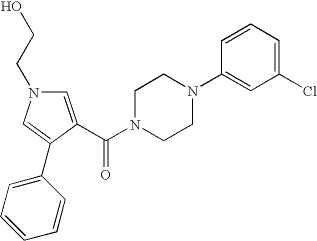 | 0.42 | 0.0115 |
| 37 | 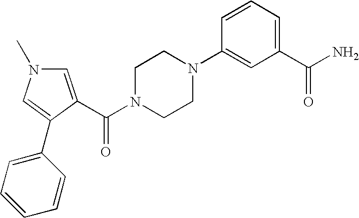 | 0.72 | 0.0149 |
| 38 | 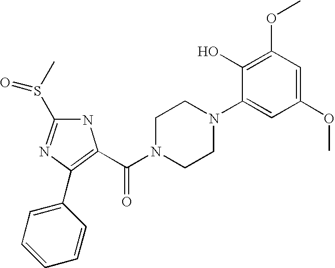 | 19.91 | 2.314 |
| 39 | 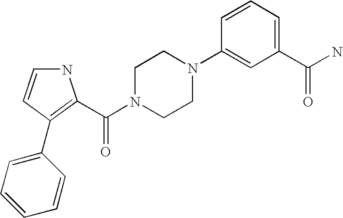 | 12.42 | 0.200 |
| 40 | 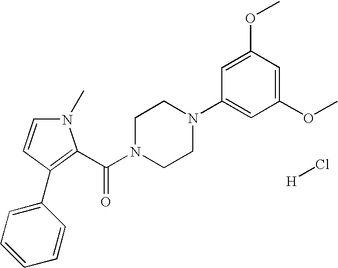 | 11.19 | 0.275 |
| 41 | 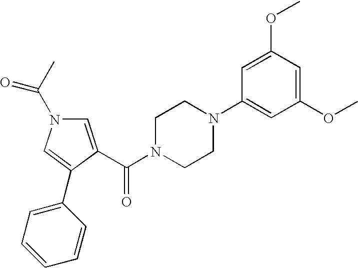 | 1.33 | 0.123 |
| 42 | 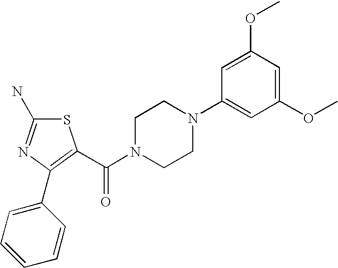 | 1.29 | 0.0016 |
| 43 | 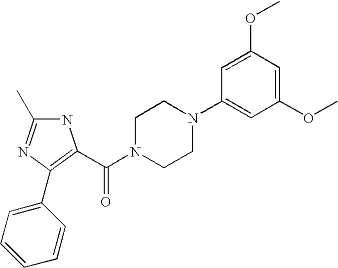 | 0.60 | 0.0071 |
| 44 | 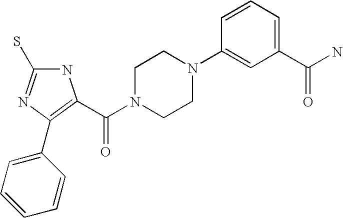 | 1.06 | |
| 45 | 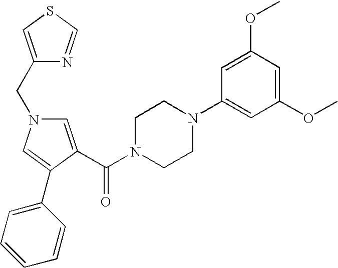 | 4.85 | n.d. |
| 46 | 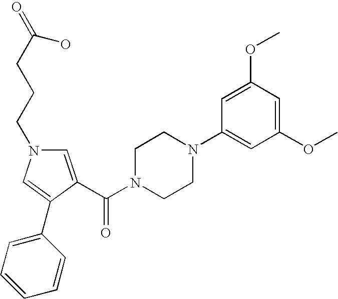 | 4.35 | 6.38 |
| 47 | 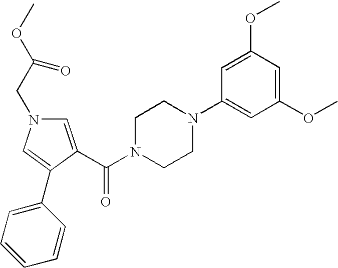 | 1.49 | 0.3269 |
| 48 | 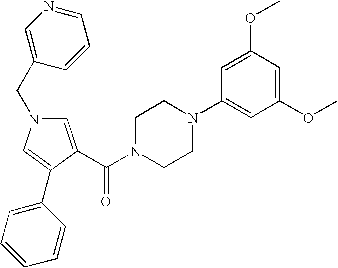 | 0.75 | 0.0511 |
| 49 | 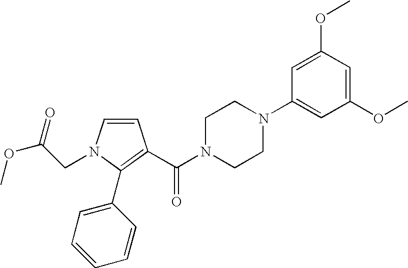 | 1.57 | n.d. |
| 50 | 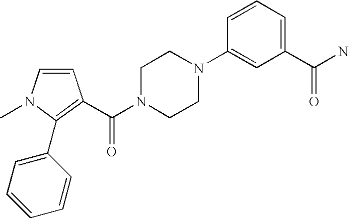 | 3.41 | 0.536 |
| 51 | 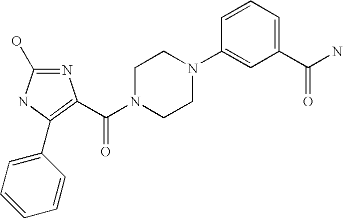 | 0.50 | 1.358 |
| 52 | 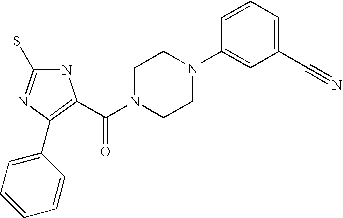 | 0.40 | 0.100 |
| 53 | 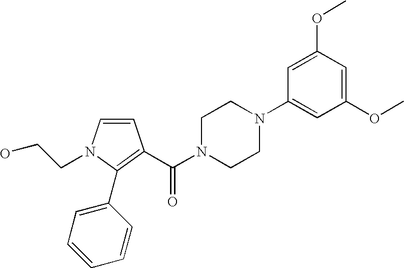 | 0.81 | 0.046 |
| 54 | 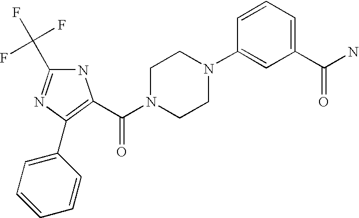 | 1.08 | 1.889 |
| 55 | 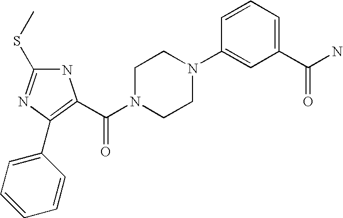 | 1.38 | 0.064 |
| 56 | 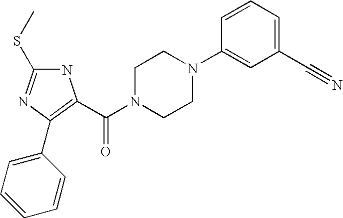 | 0.415 | 0.0017 |
| 57 | 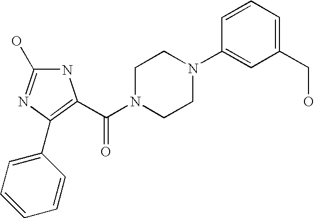 | 0.795 | 0.017 |
| 58 | 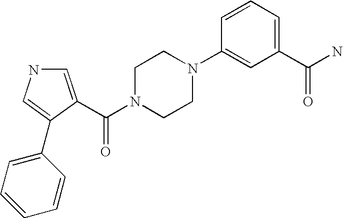 | 4.45 | 0.334 |
| 59 | 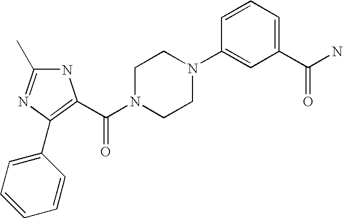 | 4.66 | 0.364 |
| 60 | 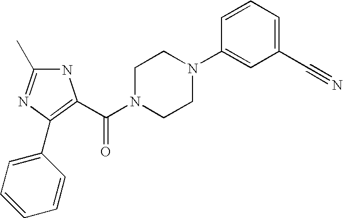 | 0.99 | 0.011 |
| 61 | 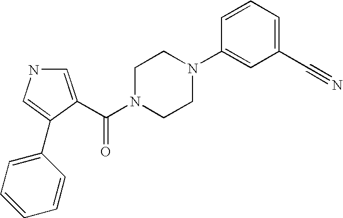 | 2.32 | 0.040 |
| 62 | 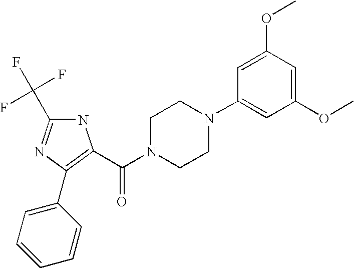 | 0.57 | 0.008 |
| 63 | 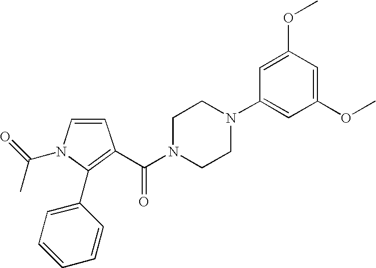 | 4.0 | n.d. |
| 64 | 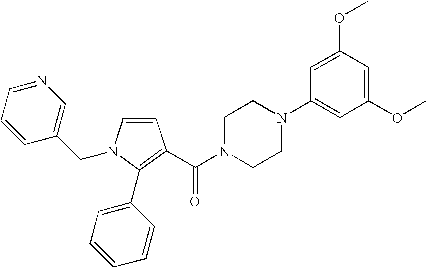 | 13.37 | n.d. |
| 65 | 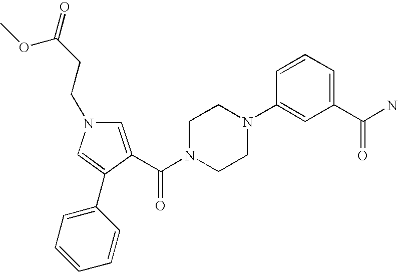 | 13.54 | n.d. |
| 66 | 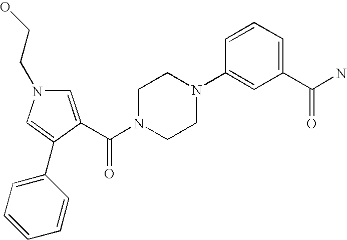 | 2.43 | 2.028 |
| 67 | 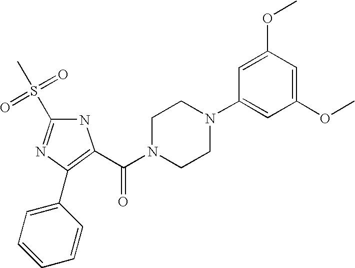 | 1.65 | 2.70 |
| 68 | 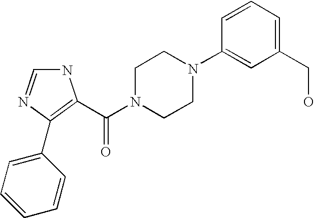 | 0.48 | 0.013 |
| 69 | 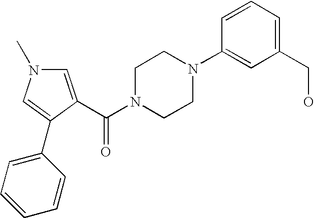 | 0.71 | 0.045 |
| 70 | 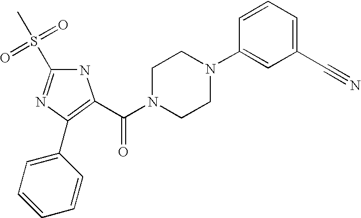 | 3.75 | n.d. |
| 71 | 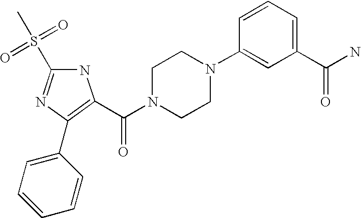 | 13.94 | n.d. |
| 72 | 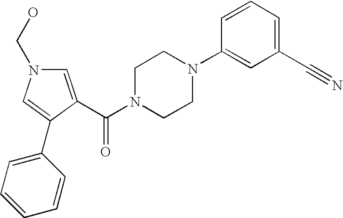 | 0.58 | 0.0295 |
| 73 | 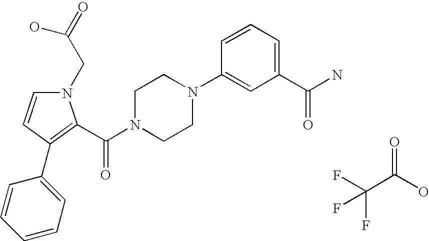 | 18.39 | 2.703 |
| 74 | 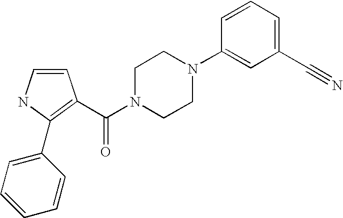 | 3.05 | 0.052 |
| 75 | 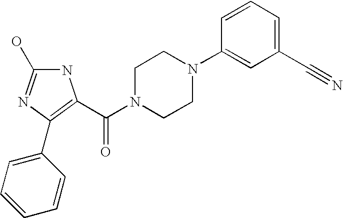 | 1.01 | 0.0115 |
| 76 | 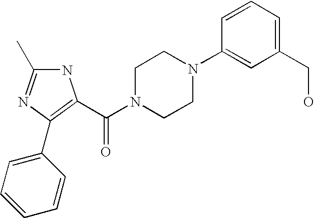 | 0.97 | 0.101 |
| 77 | 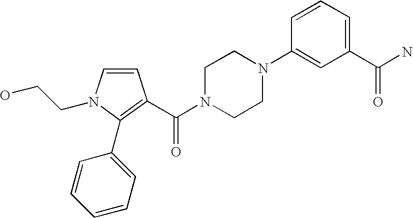 | 3.61 | n.d. |
| 78 | 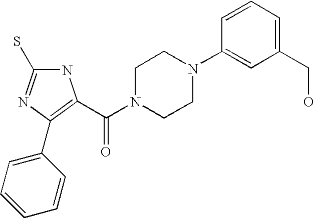 | 0.725 | 0.244 |
| 79 | 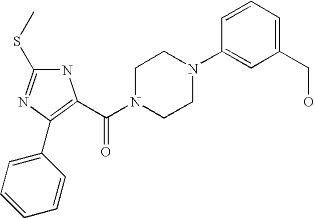 | 0.493 | 0.0117 |
| 80 | 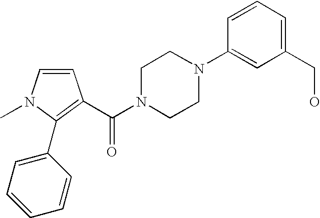 | 7.76 | n.d. |
| 81 | 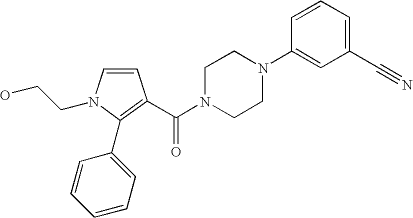 | 2.40 | 0.307 |
| 82 | 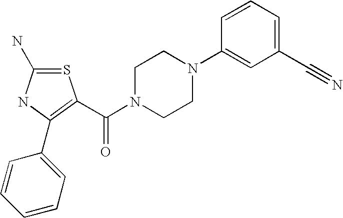 | 0.294 | n.d. |
| 83 | 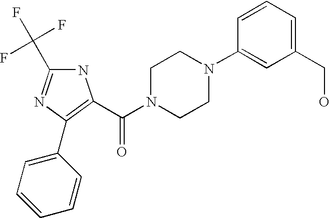 | 1.41 | n.d. |
| 84 | 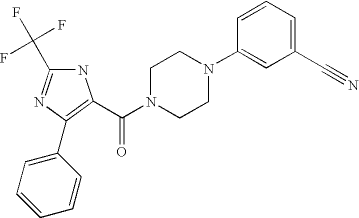 | 0.574 | n.d. |
| 85 | 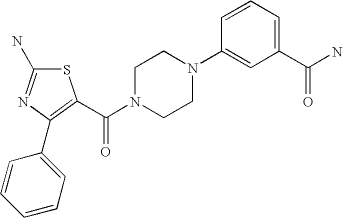 | 0.685 | n.d. |
| 86 | 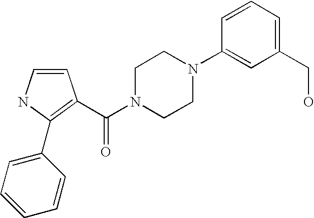 | 6.41 | n.d. |
| 87 | 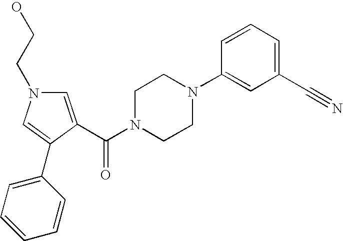 | 0.582 | n.d. |
| 88 | 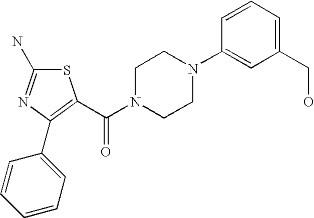 | 0.715 | n.d. |
| 89 | 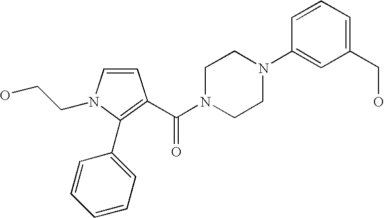 | 2.99 | n.d. |
| 90 | 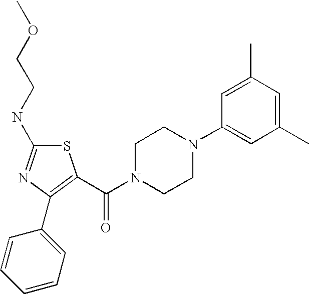 | 0.095 | n.d. |
| 91 | 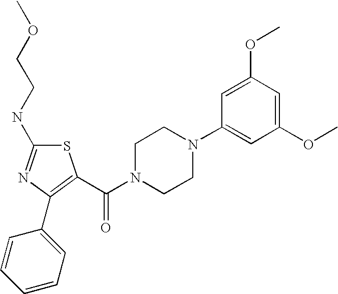 | 0.089 | n.d. |
| 92 | 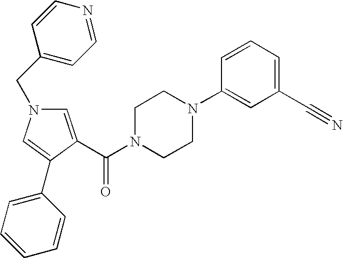 | 0.167 | n.d. |
| 93 | 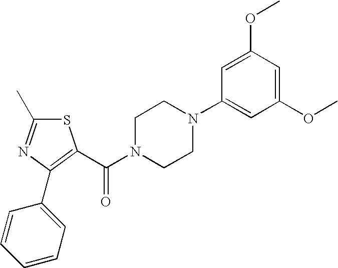 | n.d. | n.d. |
| 94 | 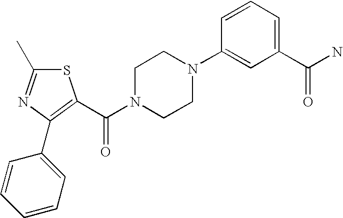 | n.d. | n.d. |
| 95 | 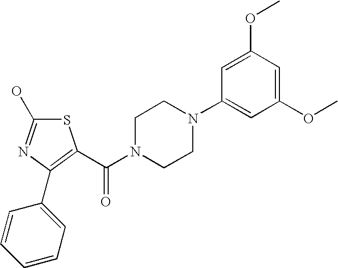 | n.d. | n.d. |
| 96 | 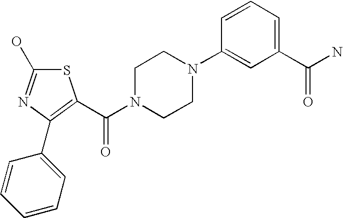 | n.d. | n.d. |
| 97 | 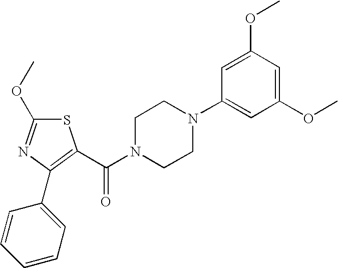 | n.d. | n.d. |
| 98 | 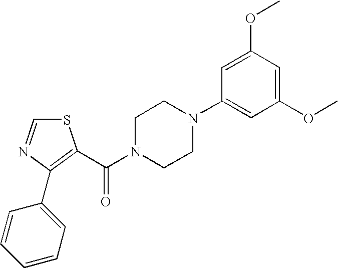 | n.d. | n.d. |
| 99 | 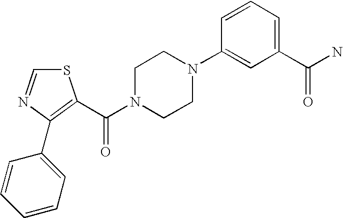 | n.d. | n.d. |
| 100 | 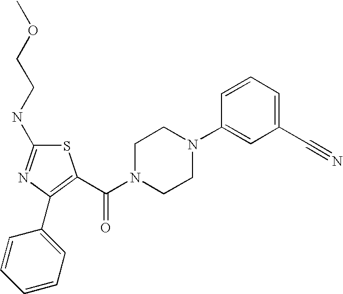 | n.d. | n.d. |
| 101 | 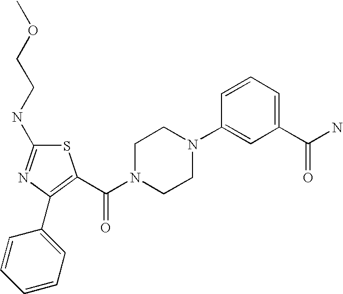 | n.d. | n.d. |

n.d.:
not determined

1. A compound of formula (I): 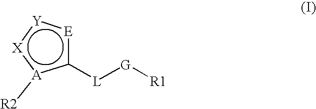
in which:
1) A is N or C;
2) L-G-R1 is chosen from 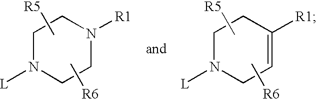
3) X and Y are chosen independently from CR3, N, NR3, O or S;
4) E is CR4, N, NR4 or S;
5) R1 and R2 are selected independently from the group consisting of aryl, heteroaryl, substituted aryl and substituted heteroaryl;
6) L is selected from the group consisting of C═O, C═S and C═N(R7);
7) R3 and R4 are selected independently from the group consisting of H, alkyl, cycloalkyl, cycloalkylene, heterocyclyl, O—R7, S—R7, SO—R7, SO₂—(R7), N(R7)(R8), halogen, aryl, heteroaryl, substituted cycloalkyl, substituted aryl, substituted heteroaryl and substituted alkyl;
8) R5 and R6 are selected independently from the group consisting of H and (C₁-C₃)alkyl;
9) R7 and R8 are selected independently from the group consisting of H, (C₁-C₃)alkyl and substituted (C₁-C₃)alkyl;
in the racemic form, enriched in one enantiomer, enriched in one diastereoisomer, its tautomers, its prodrugs and its pharmaceutically acceptable salts, with the proviso that the product of formula (I) is not one of the following compounds:

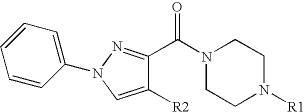

| CAS Number | R1 | R2 |
|---|---|---|
| 380442-50-2 | 2-chlorophenyl | 2-thienyl |
| 375394-90-4 | 2-methoxyphenyl | 2-thienyl |
| 380221-88-5 | 2-methoxyphenyl | 3,4-dimethoxyphenyl |
| 372106-93-9 | 2-methoxyphenyl | 3-pyridyl |
| 379266-21-4 | 3-trifluoromethylphenyl | 4-methoxyphenyl |
| 368861-17-0 | 2-methoxyphenyl | 4-chlorophenyl |
| 375395-06-5 | phenyl | 2-thienyl |
| 367512-29-6 | phenyl | phenyl |
| 367512-13-8 | 2-methoxyphenyl | 4-fluorophenyl |
| 366492-22-0 | phenyl | 4-chlorophenyl |

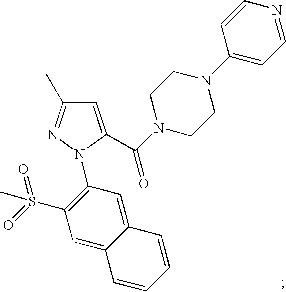

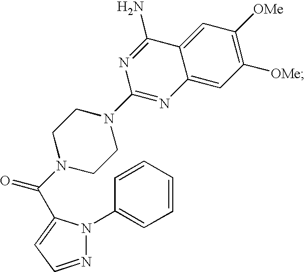

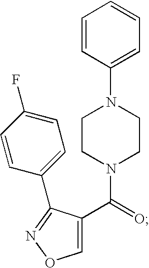

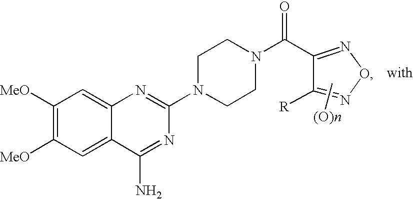

n = 0, 1 or 2 and R = phenyl.

2. The compound according to claim 1, wherein R3 is selected from the group consisting of H, alkyl, cycloalkyl, cycloalkylene, heterocyclyl, O—R7, S—R7, SO—R7, SO₂—(R7), N(R7)(R8), halogen, aryl, heteroaryl, substituted cycloalkyl, substituted aryl, substituted heteroaryl and substituted alkyl.

3. The compound according to claim 2, wherein R3 is alkyl substituted by F, OH or COOH.

4. The compound according to claim 2, wherein R3 is chosen from CF₃, CH₂OH, CH₂—CH₂OH, CH₂—CH₂—COOH and CH₂—COOH.

5. The compound according to claim 1, wherein L-G-R1 is

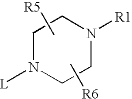

6. The compound according to claim 1, wherein E=NR4 and R4=H.

7. The compound according to claim 1, wherein R1 is selected from the group consisting of:
phenyl;
phenyl substituted by at least one radical selected from the group consisting of halogen, CF₃, CN, NO₂, (C₁-C₃)alkyl, O—R10, S—R10, N(R10)(R11), CO—O—R10, CO—N(R10)(R11), NH—CO—R10 in which R10 and R11 are chosen independently from H, (C₁-C₃)alkyl, halogenated (C₁-C₃)alkyl, (C₁-C₃)alkyl-OH, (C₁-C₃)alkyl-NH₂, (C₁-C₃)alkyl-COOH, (C₁-C₃)alkyl-OCH₃, (C₁-C₃)alkyl-NHCH₃;
pyridyl;
pyridyl substituted by at least one radical chosen from halogen, (C₁-C₃)alkyl, O—R12, S—R12, N(R12)(R13), in which R12 and R13 are chosen independently from H and (C₁-C₃)alkyl.

8. The compound according to claim 7, wherein R1 is selected from the group consisting of
phenyl substituted by (C₁-C₃)alkyl-OH, and
pyridyl substituted by (C₁-C₃)alkoxy.

9. The compound according to claim 7, wherein R1 is phenyl substituted in the 3-position by a substituent chosen from halogen, (C₁-C₃)alkyl, (C₁-C₃)alkoxy, (C₁-C₃)alkylamino, CONH₂, CO—NH—(CH₂)₂—OH, NH—CO—CH₃, 3-pyridyl, and 2- or 3-pyridyl substituted by a substituent chosen from halogen, (C₁-C₃)alkyl and (C₁-C₃)alkoxy.

10. The compound according to claim 7, wherein R1 is chosen from 2,3-disubstituted phenyl, 2,5-disubstituted phenyl, 3-substituted phenyl, 3,5-disubstituted phenyl and 3,4-disubstituted phenyl.

11. The compound according to claim 10, wherein R1 is chosen from 3-substituted phenyl, 3,5-disubstituted phenyl and 3,4-disubstituted phenyl.

12. The compound according to claim 11, wherein R1 is chosen from 3-chlorophenyl, 3,5-dimethoxyphenyl, 3-acetylaminophenyl, 3-carboxamidophenyl and 3-hydroxymethylphenyl.

13. The compound according to claim 1, wherein R1 is chosen from 2-pyridyl substituted in the 4-position, 2-pyridyl substituted in the 6-position, 2-pyridyl substituted in the 4- and 6-positions, 3-pyridyl substituted in the 2-position and 3-pyridyl substituted in the 5-position.

14. The compound according to claim 1, wherein R2 is chosen from 3-pyridyl, phenyl and phenyl substituted by a radical chosen from halogen, alkyl, O—R14, S—R14 and N(R14)(R15), in which R14 and R15 are chosen independently from H, alkyl and halogenated alkyl.

15. The compound according to claim 1, which is chosen from:
[4-(3-chlorophenyl)piperazin-1-yl](1-phenyl-1H-imidazol-2-yl)methanone,
[4-(3-chlorophenyl)piperazin-1-yl](5-phenyl-1,3-oxazol-4-yl)methanone,
[4-(3-chlorophenyl)piperazin-1-yl](4-phenyl-1H-imidazol-5-yl)methanone,
[4-(3-chlorophenyl)piperazin-1-yl](3-phenylthiophen-2-yl)methanone,
[4-(3-methoxyphenyl)piperazin-1-yl][2-(4-chlorophenyl)furan-3-yl]methanone,
[4-(3-chlorophenyl)piperazin-1-yl](3-phenyl-1H-pyrrol-2-yl)methanone,
[4-(3-chlorophenyl)piperazin-1-yl](1-methyl-3-phenyl-1H-pyrrol-2-yl)methanone, and
[4-(3,5-dimethoxyphenyl)piperazin-1-yl](3-phenyl-1H-pyrrol-2-yl)methanone.

16. The compound according to claim 1, which is chosen from:
[4-(3,5-dimethoxyphenyl)piperazin-1-yl](4-phenyl-1H-imidazol-5-yl)methanone,
[4-(pyridin-3-yl)piperazin-1-yl](4-phenyl-1H-imidazol-5-yl)methanone
[4-(3,5-dimethoxyphenyl)piperazin-1-yl](1-phenyl-1H-imidazol-5-yl)methanone,
[4-(3-acetylaminophenyl)piperazin-1-yl](4-phenyl-1H-imidazol-5-yl)methanone,
[4-(3-cyanophenyl)piperazin-1-yl](1-phenyl-1H-imidazol-5-yl)methanone,
[4-(3-cyanophenyl)piperazin-1-yl](4-phenyl-1H-imidazol-5-yl)methanone,
[4-(3-chlorophenyl)piperazin-1-yl](4-phenyl-1H-pyrrol-3-yl)methanone,
[4-(3,5-dimethoxyphenyl)piperazin-1-yl](4-phenyl-1H-pyrrol-3-yl)methanone,
[4-(3-carboxamidophenyl)piperazin-1-yl](1-phenyl-1H-imidazol-5-yl)methanone,
[4-(3,5-dimethoxyphenyl)piperazin-1-yl](1-methyl-4-phenyl-1H-pyrrol-3-yl)methanone,
[4-(3-chlorophenyl)piperazin-1-yl](2-mercapto-5-phenyl-1H-imidazol-4-yl)methanone,
[4-(3,5-dimethoxyphenyl)piperazin-1-yl](2-mercapto-5-phenyl-1H-imidazol-4-yl)methanone,
[4-(3,5-dimethoxyphenyl)piperazin-1-yl](2-phenyl-1H-pyrrol-3-yl)methanone,
[4-(3-carboxamidophenyl)piperazin-1-yl](4-phenyl-1H-imidazol-5-yl)methanone,
[4-(3,5-dimethoxyphenyl)piperazin-1-yl](2-methylsulfanyl-5-phenyl-1H-imidazol-4-yl)methanone,
[4-(3,5-dimethoxyphenyl)piperazin-1-yl](N-methyl-2-methylsulfanyl-5-phenyl-1H-imidazol-4-yl)methanone,
[4-(3-carboxamidophenyl)piperazin-1-yl](2-phenyl-1H-pyrrol-3-yl)methanone,
[4-(3-chlorophenyl)piperazin-1-yl](2-phenyl-1H-pyrrol-3-ylmethanone,
[4-(3,5-dimethoxyphenyl)piperazin-1-yl](1-methyl-2-phenyl-1H-pyrrol-3-yl)methanone,
[4-(3,5-dimethoxyphenyl)piperazin-1-yl](2-hydroxy-5-phenyl-1H-imidazol-4-yl)methanone,
[4-(3-methoxyphenyl)piperazin-1-yl](4-phenyl-1H-imidazol-5-yl)methanone,
[4-(3-difluoromethoxyphenyl)piperazin-1-yl](5-phenyl-1H-imidazol-4-yl)methanone,
[4-(3-chlorophenyl)piperazin-1-yl][1-(2-dimethylaminoethyl)-4-phenyl-1H-pyrrol-3-yl]methanone,
3-{3-[4-(3-chlorophenyl)piperazin-1-ylcarbonyl]-4-phenylpyrrol-1-yl}propionic acid,
[4-(3,5-dimethoxyphenyl)piperazin-1-yl](2-methanesulfinyl-5-phenyl-1H-imidazol-4-yl)methanone,
methyl 3-{3-[4-(3-chlorophenyl)piperazin-1-ylcarbonyl]-4-phenylpyrrol-1-yl}-propionate,
[4-(3,5-dimethoxyphenyl)piperazin-1-yl](1-hydroxymethyl-4-phenyl-1H-pyrrol-3-yl)methanone,
[4-(3,5-dimethoxyphenyl)piperazin-1-yl][1-(2-hydroxyethyl)-4-phenyl-1H-pyrrol-3-yl]methanone,
3-[4-(1-methyl-4-phenyl-1H-pyrrol-3-ylcarbonyl)piperazin-1-yl]benzamide,
[4-(2-hydroxy-3,5-dimethoxyphenyl)piperazin-1-yl](2-methanesulfinyl-5-phenyl-1H-imidazol-4-yl)methanone,
3-[4-(3-phenyl-1H-pyrrol-2-ylcarbonyl)piperazin-1-yl]benzamide,
[4-(3,5-dimethoxyphenyl)piperazin-1-yl](1-methyl-3-phenyl-1H-pyrrol-2-yl)methanone,
1-{3-[4-(3,5-dimethoxyphenyl)piperazin-1-ylcarbonyl]-4-phenylpyrrol-1-yl}-ethanone,
(2-amino-4-phenylthiazol-5-yl)[4-(3,5-dimethoxyphenyl)piperazin-1-yl]-methanone,
[4-(3,5-dimethoxyphenyl)piperazin-1-yl](2-methyl-5-phenyl-3H-imidazol-4-yl)methanone,
3-[4-(2-mercapto-4-phenyl-1H-imidazol-5-ylcarbonyl)piperazin-1-yl]-benzamide,
[4-(3,5-dimethoxyphenyl)piperazin-1-yl][1-(thiazol-4-yl)methyl-4-phenyl-1H-pyrrol-3-yl]methanone,
4-{3-[4-(3,5-dimethoxyphenyl)piperazin-1-yl]carbonyl-4-phenyl-1H-pyrrol-1-yl}-butanoic acid,
2-{3-[4-(3,5-dimethoxyphenyl)piperazin-1-yl]carbonyl-4-phenyl-1H-pyrrol-1-yl}-acetic acid,
[4-(3,5-dimethoxyphenyl)piperazin-1-yl)[1-(pyridin-3-yl)methyl-4-phenyl-1H-pyrrol-3-yl]methanone,
methyl 2-{3-[4-(3,5-dimethoxyphenyl)piperazin-1-yl]carbonyl-2-phenyl-1H-pyrrol-1-yl}acetate,
3-[4-(1-methyl-2-phenyl-1H-pyrrol-3-ylcarbonyl)piperazin-1-yl]benzamide,
3-[4-(2-hydroxy-4-phenyl-1H-imidazol-5-ylcarbonyl)piperazin-1-yl]benzamide,
3-[4-(2-mercapto-4-phenyl-1H-imidazol-5-ylcarbonyl)piperazin-1-yl]-benzonitrile,
[4-(3,5-dimethoxyphenyl)piperazin-1-yl][1-(2-hydroxyethyl)-2-phenyl-1H-pyrrol-3-yl]methanone,
3-[4-(2-trifluoromethyl-4-phenyl-1H-imidazol-5-ylcarbonyl)piperazin-1-yl]benzamide,
3-[4-(2-methylsulfanyl-4-phenyl-1H-imidazol-5-ylcarbonyl)piperazin-1-yl]benzamide,
3-[4-(2-methylsulfanyl-4-phenyl-1H-imidazol-5-ylcarbonyl)piperazin-1-yl]benzonitrile,
[4-(3-hydroxymethylphenyl)piperazin-1-yl](2-hydroxy-4-phenyl-1H-imidazol-5-yl)methanone,
3-[4-(4-phenyl-1H-pyrrol-3-ylcarbonyl)piperazin-1-yl]benzamide,
3-[4-(2-methyl-5-phenyl-1H-imidazol-4-ylcarbonyl)piperazin-1-yl]benzamide,
3-[4-(2-methyl-5-phenyl-1H-imidazol-4-ylcarbonyl)piperazin-1-yl]benzonitrile,
3-[4-(4-phenyl-1H-pyrrol-3-ylcarbonyl)piperazin-1-yl]benzonitrile,
[4-(3,5-dimethoxyphenyl)piperazin-1-yl](2-trifluoromethyl-4-phenyl-1H-imidazol-5-yl)methanone,
[4-(3,5-dimethoxyphenyl)piperazin-1-yl](1-acetyl-2-phenyl-1H-pyrrol-3-yl)methanone,
[4-(3,5-dimethoxyphenyl)piperazin-1-yl][1-(3-pyridyl)methyl-2-phenyl-1H-pyrrol-3-yl]methanone,
3-[4-(2-methoxycarbonylmethyl-4-phenyl-1H-pyrrol-3-carbonyl)piperazin-1-yl]benzamide,
3-[4-(1-hydroxyethyl-4-phenyl-1H-pyrrol-3-ylcarbonyl)piperazin-1-yl]benzamide,
[4-(3,5-dimethoxyphenyl)piperazin-1-yl](2-methylsulfonyl-4-phenyl-1H-imidazol-5-yl)methanone,
[4-(3-hydroxymethylphenyl)piperazin-1-yl](4-phenyl-1H-imidazol-5-yl)methanone,
[4-(3,5-hydroxymethylphenyl)piperazin-1-yl](1-methyl-4-phenyl-1H-pyrrol-3-yl)methanone,
3-[4-(2-methylsulfonyl-4-phenyl-1H-imidazol-4-ylcarbonyl)piperazin-1-yl]benzonitrile,
3-[4-(2-methylsulfonyl-4-phenyl-1H-imidazol-4-ylcarbonyl)piperazin-1-yl]benzamide,
3-[4-(1-hydroxymethyl-4-phenyl-1H-pyrrol-3-ylcarbonyl)piperazin-1-yl]benzonitrile,
{2-[4-(3-carbamoylphenyl)piperazin-1-ylcarbonyl]-3-phenylpyrrol-1-yl}acetic acid,
3-[4-(2-phenyl-1H-pyrrol-3-ylcarbonyl)piperazin-1-yl]benzonitrile,
3-[4-(2-hydroxy-4-phenyl-1H-imidazol-5-ylcarbonyl)piperazin-1-yl]benzonitrile,
[4-(3-hydroxymethylphenyl)piperazin-1-yl](2-methyl-4-phenyl-1H-imidazol-5-yl]methanone,
3-[4-(1-hydroxyethyl-2-phenyl-1H-pyrrol-3-ylcarbonyl)piperazin-1-yl]benzamide,
[4-(3-hydroxymethylphenyl)piperazin-1-yl](2-mercapto-4-phenyl-1H-imidazol-5-yl)methanone,
[4-(3-hydroxymethylphenyl)piperazin-1-yl](2-methylsulfanyl-4-phenyl-1H-imidazol-5-yl)methanone,
[4-(3-hydroxymethylphenyl)piperazin-1-yl](1-methyl-2-phenyl-1H-pyrrol-3-yl)methanone,
3-[4-(1-hydroxyethyl-2-phenyl-1H-pyrrol-3-ylcarbonyl)piperazin-1-yl]benzonitrile,
3-[4-(2-amino-4-phenylthiazol-5-ylcarbonyl)piperazin-1-yl]benzonitrile,
[4-(3-hydroxymethylphenyl)piperazin-1-yl](2-trifluoromethyl-4-phenyl-1H-imidazol-5-yl)methanone,
3-[4-(2-trifluoromethyl-4-phenyl-1H-imidazol-5-ylcarbonyl)piperazin-1-yl]benzonitrile,
3-[4-(2-amino-4-phenylthiazol-5-ylcarbonyl)piperazin-1-yl]benzamide,
[4-(3-hydroxymethylphenyl)piperazin-1-yl](2-phenyl-1H-pyrrol-3-yl)methanone,
3-[4-(1-hydroxyethyl-4-phenyl-1H-pyrrol-3-ylcarbonyl)piperazin-1-yl]benzonitrile,
[4-(3-hydroxymethylphenyl)piperazin-1-yl](2-amino-4-phenylthiazol-5-yl)methanone,
[4-(3-hydroxymethylphenyl)piperazin-1-yl](1-hydroxyethyl-2-phenyl-1H-pyrrol-5-yl)methanone,
[4-(3,5-dimethylphenyl)piperazin-1-yl][2-(2-methoxyethyl)amino-4-phenylthiazol-5-yl]methanone,
[4-(3,5-dimethoxyphenyl)piperazin-1-yl][2-(2-methoxyethyl)amino-4-phenylthiazol-5-yl]methanone,
3-{4-[1-(pyridin-3-yl)methyl-4-phenyl-1H-pyrrol-3-ylcarbonyl]piperazin-1-yl}benzonitrile,
[4-(3,5-dimethoxyphenyl)piperazin-1-yl](2-methyl-4-phenylthiazol-5-yl)methanone,
3-[4-(2-methyl-4-phenylthiazol-5-ylcarbonyl)piperazin-1-yl]benzamide,
[4-(3,5-dimethoxyphenyl)piperazin-1-yl](2-hydroxy-4-phenylthiazol-5-yl)methanone,
3-[4-(2-hydroxy-4-phenylthiazol-5-ylcarbonyl)piperazin-1-yl]benzamide,
[4-(3,5-dimethoxyphenyl)piperazin-1-yl](2-methoxy-4-phenylthiazol-5-yl)methanone,
[4-(3,5-dimethoxyphenyl)piperazin-1-yl](4-phenylthiazol-5-yl)methanone,
3-[4-(2-hydroxy-4-phenylthiazol-5-ylcarbonyl)piperazin-1-yl]benzamide,
3-{4-[2-(2-methoxyethyl)amino-4-phenylthiazol-5-ylcarbonyl]piperazin-1-yl}benzonitrile, and
3-{4-[2-(2-methoxyethyl)amino-4-phenylthiazol-5-ylcarbonyl]piperazin-1-yl}benzamide.

17. A pharmaceutical composition comprising a compound of formula (I): 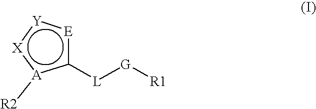
in which:
1) A is N or C;
2) L-G-R1 is chosen from 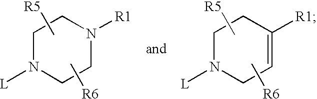
3) X and Y are chosen independently from CR3, N, NR3, O or S;
4) E is CR4, N, NR4 or S;
5) R1 and R2 are selected independently from the group consisting of aryl, heteroaryl, substituted aryl and substituted heteroaryl;
6) L is selected from the group consisting of C═O, C═S and C═N(R7);
7) R3 and R4 are selected independently from the group consisting of H, alkyl, cycloalkyl, cycloalkylene, heterocyclyl, O—R7, S—R7, SO—R7, SO₂—(R7), N(R7)(R8), halogen, aryl, heteroaryl, substituted cycloalkyl, substituted aryl, substituted heteroaryl and substituted alkyl;
8) R5 and R6 are selected independently from the group consisting of H and (C₁-C₃)alkyl;
9) R7 and R8 are selected independently from the group consisting of H, (C₁-C₃)alkyl and substituted (C₁-C₃)alkyl;
in the racemic form, enriched in one enantiomer, enriched in one diastereoisomer, its tautomers, its prodrugs and its pharmaceutically acceptable salts, with the proviso that the product of formula (1) is not one of the following compounds:

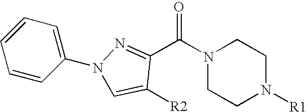

| CAS Number | R1 | R2 |
|---|---|---|
| 380442-50-2 | 2-chlorophenyl | 2-thienyl |
| 375394-90-4 | 2-methoxyphenyl | 2-thienyl |
| 380221-88-5 | 2-methoxyphenyl | 3,4-dimethoxyphenyl |
| 372106-93-9 | 2-methoxyphenyl | 3-pyridyl |
| 379266-21-4 | 3-trifluoromethylphenyl | 4-methoxyphenyl |
| 368861-17-0 | 2-methoxyphenyl | 4-chlorophenyl |
| 375395-06-5 | phenyl | 2-thienyl |
| 367512-29-6 | phenyl | phenyl |
| 367512-13-8 | 2-methoxyphenyl | 4-fluorophenyl |
| 366492-22-0 | phenyl | 4-chlorophenyl |

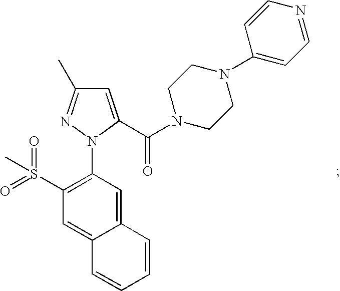

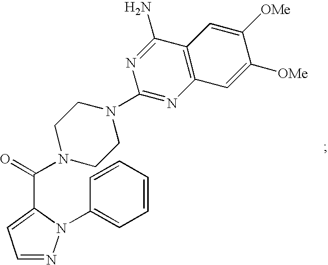

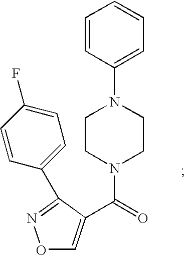

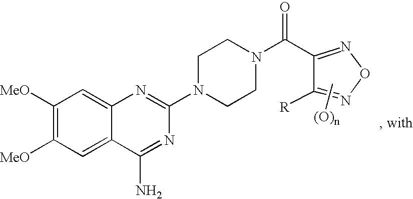

n = 0, 1 or 2 and R = phenyl;
in combination with a pharmaceutically acceptable excipient.

18. A method of treating a pathological condition treatable by inhibiting tubulin polymerization, comprising administering to a subject in need thereof a therapeutically effective amount of a compound of formula (I): 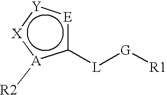
in which:
1) A is N or C,
2) L-G-R1 is chosen from 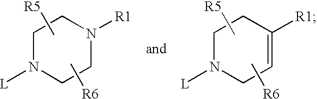
3) X and Y are chosen independently from CR3, N, NR3, O or S;
4) E is CR4, N, NR4 or S;
5) R1 and R2 are selected independently from the group consisting of aryl, heteroaryl, substituted aryl and substituted heteroaryl;
6) L is selected from the group consisting of C═O, C═S and C═N(R7);
7) R3 and R4 are selected independently from the group consisting of H, alkyl, cycloalkyl, cycloalkylene, heterocyclyl, O—R7, S—R7, SO—R7, SO₂—(R7), N(R7)(R8), halogen, aryl, heteroaryl, substituted cycloalkyl, substituted aryl, substituted heteroaryl and substituted alkyl;
8) R5 and R6 are selected independently from the group consisting of H and (C₁-C₃)alkyl;
9) R7 and R8 are selected independently from the group consisting of H, (C₁-C₃)alkyl and substituted (C₁-C₃)alkyl;
in the racemic form, enriched in one enantiomer, enriched in one diastereoisomer, its tautomers, its prodrugs and its pharmaceutically acceptable salts, with the proviso that the product of formula (I) is not one of the following compounds:

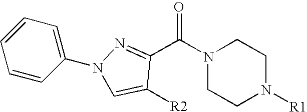

| CAS Number | R1 | R2 |
|---|---|---|
| 380442-50-2 | 2-chlorophenyl | 2-thienyl |
| 375394-90-4 | 2-methoxyphenyl | 2-thienyl |
| 380221-88-5 | 2-methoxyphenyl | 3,4-dimethoxyphenyl |
| 372106-93-9 | 2-methoxyphenyl | 3-pyridyl |
| 379266-21-4 | 3-trifluoromethylphenyl | 4-methoxyphenyl |
| 368861-17-0 | 2-methoxyphenyl | 4-chlorophenyl |
| 375395-06-5 | phenyl | 2-thienyl |
| 367512-29-6 | phenyl | phenyl |
| 367512-13-8 | 2-methoxyphenyl | 4-fluorophenyl |
| 366492-22-0 | phenyl | 4-chlorophenyl |

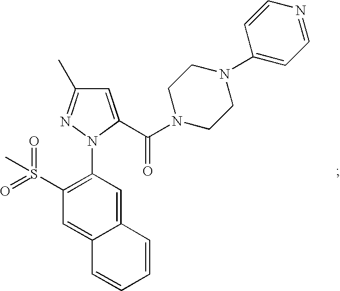

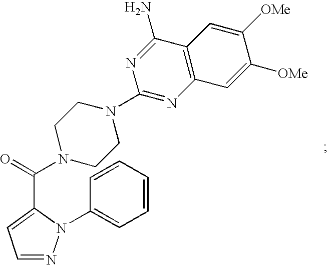

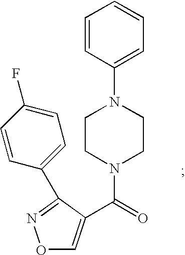

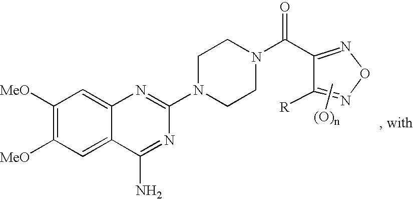

n = 0, 1 or 2 and R = phenyl.

19. The method according to claim 18, wherein the pathological condition is caused by a proliferaton of tumor cells.

20. The method according to claim 18, wherein the pathological condition is cancer.
